(19) United States
(12) Patent Application Publication
(10) Pub. No.: US 2025/0215452 A1
(43) Pub. Date: Jul. 3, 2025

(54) Adenoviral Mediated Targeting of Activated Immune Cells

(71) Applicants: Universität Zürich, Zürich (CH); Vector Biopharma AG, Basel (CH)

(72) Inventors: Patrick Christian FREITAG, Zürich (CH); Andreas PLÜCKTHUN, Zürich (CH); Meike KAULFUSS, Zürich (CH); Obinna CHIJIOKE, Zürich (CH); Bruno Loureiro CADILHA, Weil am Rhein (DE); Nicole KIRCHHAMMER, Basel (CH); Vasileios ATSAVES, Basel (CH); Sheena Nichole SMITH, Bubendorf (CH)

(21) Appl. No.: 18/848,589

(22) PCT Filed: Mar. 24, 2023
(86) PCT No.: PCT/EP2023/057637

§ 371 Date: Sep. 19, 2024

(30) Foreign Application Priority Data
Mar. 25, 2022 (EP) 22164337.2
Dec. 1, 2022 (EP) 22210726.0

(51) Int. Cl. C12N 15/86; C07K 14/55; C07K 16/28

(52) U.S. Cl. CPC C12N 15/86; C07K 14/55; C07K 16/2809; C07K 16/2818; C07K 2317/565; C07K 2317/622; C07K 2319/30; C12N 2710/10343; C12N 2710/10345

(57) ABSTRACT

Disclosed herein are recombinant adenoviruses, recombinant proteins and trimeric proteins that are useful for the transduction of immune cells, such as T cells or NK cells. The present invention provides a versatile and highly specific system that is useful for numerous purposes, including the development of novel therapeutic approaches. The present invention makes use of specially designed adapter molecules that enable the specific targeting of immune cells, such as T cells or NK cells, which strongly increases transduction efficiencies, in particular for in vitro transduction of immune cells.

### FIELD OF THE INVENTION

Disclosed herein are recombinant adenoviruses, recombinant proteins and trimeric proteins that are useful for the transduction of immune cells, such as T cells or NK cells. The present invention provides a versatile and highly specific system that is useful for numerous purposes, including the development of novel therapeutic approaches.

### BACKGROUND

Decades of research have aimed to genetically engineer cells for scientific and therapeutic purposes (Cell (2017) 168:724-740; Nature (2018) 559:405-9). Vast advances in cell therapy have been made using recombinant viral vectors, or adeno-associated vectors (Nature Methods (2019) 16:247-54; Science (2015) 348:62-8). These advances lead to a better understanding of required and distinct molecular mechanisms of action for effective therapeutics. Such molecular mechanisms benefit from precise tunability and controllability at the DNA level, thus leading to an increase of the required DNA complexity and unavoidably increasing the demand of base pairs to encode for said complexity (Molecular Cancer (2019) 18:125). Currently used vectors do not allow insertion of large DNA payloads, being limited to payloads up around 7 kb (thousand base pairs) DNA. To circumvent viral production, non-viral delivery methods can be used for screening purposes. Circumventing the toxicity of the DNA, linear DNA transfection (Nature (2018) 559:405-9; Vol. 23, Nature Medicine (2017) 23:415-23), RNA transfection (Molecular Therapy (2006) 13:151-9) or RNA-containing lipid nanoparticles (LNPs) (Science (2022) 375:91-6) have been developed. However, not only remains the limitation of the deliverable DNA size as an obstacle, an incorporation of various payloads into a single vector with sufficient size also guarantees defined DNA ratios in the target cell and successful delivery of all fragments. Ideally a vector of choice would combine large packaging capacities in combination with effective transduction and reported safe delivery in mice and humans, allowing targeted transduction in an in vitro and in vivo setting.

Human adenovirus serotype 5 vectors (HAdV-C5) can not only be modified to high-capacity vectors (HC-AdVs) (36 kb packaging size) but are also validated for safety in various clinical trials and animal models (Virus Genes (2017) 53:684-691; J Hematol Oncol (2020) 13:84; Adenoviral Gene Therapy (2002) 7, 46-59). Increasing the safety of human application further, HAdV genomes exist extra-chromosomally, minimizing the risk of unwanted insertional mutation and germline transmission. However, if desired, HC-AdVs have been reported as a single delivery entity combining donor DNA and a Cas9 system enabling site-specific insertion and deletion (Molecular Therapy: Methods and Clinical Development (2020) 17:441-7), qualifying HAdV-C5 to an ideal and versatile vector overcoming limitations of currently available methods for the engineering of immune cells, such as for example immune cells.

Cellular entry of HAdV-C5 vectors can happen efficiently in a receptor-specific manner, but also at lower rates through unspecific and poorly characterized mechanisms. Receptor-specific cellular entry is mediated mostly through contact by the homotrimeric knob protein to the coxsackie and adenoviral receptors (CAR) and subsequent interaction with RGD binding integrins on the cell membrane. Both CAR and RGD binding integrins are unproven to be expressed by immune cells at high levels (Ann Rev Virol (2019) 6:177-97; J Cell Sci (2003) 116:4695-705; Int J Mol Sci (2018) 19:485; J Virol (1995) 69:2257-63). This renders immune cells, such as T cells or NK cells, hard to transduce by HAdV-C5 without engineering of vector or cells (PLOS ONE (2017) 12:1-12). In the T cell engineering field, for instance, this has led to expression of either CAR (Proc Natl Acad Sci USA (1998) 95:13159-64) or αVβ3/5 integrins (J Virol (1995) 69:2257-63) making T cells more susceptible for adenoviral infections and the generation of chimeric adenoviral vectors such as Ad5/35 interacting with CD46 expressed on all nucleated human cells (Exp Hematol (2004) 32:536-46; International J Biochem Cell Biol (1999) 31:1255-60).

However, achieving transduction of immune cells, such as T cells or NK cells, by HAdV-C5 vectors in genetically unmodified cells would allow its application in various scientific and clinical settings. We hypothesized that a previously described trimeric adapter technology (J Mol Biol (2011) 405:410-26; Proc Natl Acad Sci USA (2013) 110: E869-77) could be engineered to generate an easily applicable method to enable adenovirus mediated transduction, allowing universal usage with all currently developed adenoviral vectors. Additionally, direct in vivo engineering could be possible while being compatible with the previously reported shield design, increasing tissue specificity while reducing liver uptake.

The present invention makes use of specially designed adapter molecules that enable the specific targeting of immune cells, such as T cells or NK cells, which strongly increases transduction efficiencies, in particular for in vitro transduction of immune cells.

### SUMMARY OF THE INVENTION

The present disclosure provides methods for the transduction of immune cells, such as T cells or NK cells. Methods are provided which allow for the efficient transduction of activated immune cells. Methods are also provided which allow for the efficient transduction of non-activated immune cells.

The present disclosure relates to a method for the transduction of immune cell, said method comprising
a. contacting said immune cell at about the same time with
   i. a recombinant adenovirus, and
   ii. an agent capable of activating said immune cells, and
b. incubating the mixture obtained in step a. for a time sufficient for transduction of said immune cells.

The present disclosure also relates to a method for the transduction of immune cell, said method comprising
a. contacting said immune cell at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said immune cells. In certain embodiments, said functional interleukin 2 polypeptide is displayed on the knob of said adenovirus. In certain embodiments said functional interleukin 2 polypeptide comprises the amino acid sequence of SEQ ID No. 4 or 5.

In certain embodiments, said functional interleukin 2 polypeptide is comprised in a recombinant protein comprising from the N- to the C-terminus
a) said functional interleukin 2 polypeptide,
b) a designed ankyrin repeat domain which binds to the knob of the adenovirus, and
c) a trimerization domain.

In certain embodiments, said designed ankyrin repeat domain that binds to a knob of an adenovirus comprises the amino acid sequence of SEQ ID No. 2.

In certain embodiments, said trimerization domain is or is derived from the capsid protein SHP of lambdoid phage 21. In certain embodiments, said trimerization domain comprises the amino acid sequence of SEQ ID No. 1. In certain embodiments, said trimerization domain is derived from collagen sources.

In certain embodiments, said immune cells are selected from T cells or NK cells]. In certain embodiments said immune cells are T cells. In certain embodiments, said T cells are CD4-positive T cells. In certain embodiments, said T cells are CD8-positive T cells. In certain embodiments, said immune cells are CD4-positive T cells which are CD25^{high} and CD127^{low}. In certain embodiments, said immune cells are T regulatory cell (Treg cells). In certain embodiments, said immune cells are CD25^{high} and CD127^{low} T regulatory cell (Treg cells). In certain embodiments said immune cells are NK cells.

In certain embodiments, said agent capable of activating said immune cells is selected from DynaBeads (e.g. Dynabeads™ Human T-Expander CD3/CD28, Gibco, Catalogue number 11141D; Dynabeads™ Human T-Expander CD3/CD28/CD137, Gibco, Catalogue number 11162D), TransAct (T Cell TransAct™ human, Miltenyi, Catalogue number 130-111-160), Polybrene and PMA (1-Methoxy-2-propylacetat). In certain embodiments, said agent capable of activating said immune cells is TransAct. In certain embodiments, said agent capable of activating said immune cells is GMP compliant. In certain embodiments, said agent capable of activating said immune cells is GMP compliant TransAct.

In certain embodiments, said immune cells, said recombinant adenovirus displaying a functional interleukin 2 polypeptide, and said agent capable of activating said immune cells are contacted at about the same time.

In certain embodiments, said immune cells, said recombinant adenovirus displaying a functional interleukin 2 polypeptide, and said agent capable of activating said immune cells are contacted simultaneously.

In certain embodiments said method is performed in vitro. In certain embodiments said methods is performed through extracorporeal apheresis-based devices.

The present disclosure also provides recombinant adenoviruses, sets of recombinant adenoviruses, recombinant proteins and trimeric proteins that are useful for the transduction of immune cells, such as T cells or NK cells.

The present disclosure provides a recombinant adenovirus or a set of recombinant adenoviruses displaying
a) an antigen-binding moiety specifically binding to CD3,
b) an antigen-binding moiety specifically binding to CD28, and/or
c) a functional interleukin 2 polypeptide.

The antigen-binding moiety specifically binding to CD3 may be a single chain antibody specifically binding to CD3, for example a single chain antibody comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17).

The antigen-binding moiety specifically binding to CD28 may be a single chain antibody, for example a single chain antibody comprising an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24).

The functional interleukin 2 polypeptide may be a polypeptide comprising the amino acid sequence of SEQ ID No. 4.

The antigen-binding moiety specifically binding to CD3, the antigen-binding moiety specifically binding to CD28, and the functional interleukin 2 polypeptide are displayed on the knob of an adenovirus.

The recombinant proteins of the present disclosure may comprise from the N- to the C-terminus
a) an antigen-binding moiety specifically binding to CD3, an antigen-binding moiety specifically binding to CD28, or a functional interleukin 2 polypeptide,
b) a designed ankyrin repeat domain which binds to the knob of the adenovirus, and
c) a trimerization domain.

The designed ankyrin repeat domain that binds to a knob of an adenovirus may comprise the amino acid sequence of SEQ ID No. 2.

The trimerization domain may be or may be derived from the capsid protein SHP of lambdoid phage 21, for example, a trimerization domain comprising the amino acid sequence of SEQ ID No. 1.

The present disclosure also provides trimeric proteins consisting of three recombinant proteins of aforementioned architecture. The three recombinant proteins comprised in said trimeric protein may be identical. The three recombinant proteins comprised in said trimeric protein may also be different.

The present disclosure also provides nucleic acids encoding the recombinant proteins described herein.

The present disclosure also provides the use of the recombinant adenoviruses, the set of recombinant adenoviruses, the recombinant proteins or the trimeric proteins disclosed herein for the transduction of immune cells, such as T cells or NK cells.

The present disclosure also provides the use of the recombinant adenoviruses, the set of recombinant adenoviruses, the recombinant proteins or the trimeric proteins disclosed herein for the use in medicine.

The present disclosure also provides a eukaryotic cell expressing or producing a recombinant adenovirus or the set of recombinant adenoviruses disclosed herein.

### FIGURE LEGENDS

FIG. 1 shows the GFP transduction efficacies for non-activated T cells with adenoviruses encoding for GFP displaying various adapters. All experimental conditions tested led to a transduction efficacy of less than 3%.

FIG. 2 shows the percentage of T cells expressing GFP when T cells were incubated with adenovirus encoding for GFP simultaneous to the activation of the T cells. T cells were effectively transfected with the IL-2 retargeted adapter and event with the untargeted adenovirus.

FIG. 3 shows the MFI (median fluorescence intensity) of T cells expressing GFP when T cells were incubated with adenovirus encoding for GFP simultaneous to the activation of the T cells.

FIG. 4 compares three treatment groups. In group A, T cells were activated with Dynabeads for 48 hours prior to the addition of adenoviruses encoding for GFP displaying the IL-2 adapter. In group B, Dynabeads and adenoviruses encoding for GFP were added simultaneously at time point zero. In group C, Dynabeads and adenoviruses encoding for GFP were also added simultaneously at time point zero, with the addition of additional adenoviruses encoding for GFP after 48 hours. Adding the IL2-retargeting adapter simultaneously to the activation of T cells significantly increase the transduction efficiency.

FIG. 5 shows that for efficient transduction of T cells a simultaneous infection and activation of the T cells is the most important parameter.

FIGS. 6 and 7 show that a high transduction efficacy is dependent on adenoviruses encoding for GFP displaying the IL-2 adapter. Furthermore, longer incubation times lead to a significant increase of the transduction efficacy.

FIG. 8 shows that the efficient transduction with the IL-2 adapter can be observed for T cells and for PBMCs. It hence makes no difference if T cells are infected directly of if PBMCs are used.

FIG. 9 shows that the transgene (GFP) is readily detectable in cells transduced with adenoviruses displaying the IL-2 adapter and remains detectable for at least two weeks after transduction.

FIG. 10 shows that the Dynabead-to-T cell ratio and the adapter molecule-to-fiber knob ratio has little effect on the transduction efficacy.

FIG. 11 shows that transduction efficiency of above 95% can be achieved with optimized experimental conditions.

FIG. 12 shows that the number of genomic particles per cell and the adapters to fiber knobs did not have a significant impact on T cell viability.

FIG. 13 shows that neither the adapter, nor the virus skews the cell population towards CD4-positive or CD8-positive T cells.

FIG. 14 shows that CD4-positive T cells were slightly better transduced compared to CD8-positive T cells.

FIG. 15 shows that neither the adapter, nor the virus skews the cell population towards conventional T cells or regulatory T cells.

FIG. 16 shows that naïve and central memory are the predominant compartments after T cell transduction.

FIG. 17 shows the percentage of Treg cells expressing GFP when Treg cells were incubated with adenovirus encoding for GFP simultaneous to the activation of the Treg cells. Treg cells were effectively transfected with the IL-2 retargeted adapter.

FIG. 18 shows the MFI of Treg cells expressing GFP when T cells were incubated with adenovirus encoding for GFP simultaneous to the activation of the T cells.

FIG. 19 shows the transduction efficiency of first-generation adenoviruses displaying various targeting moieties (shown as A28, A29, etc) compared to untargeted adenoviruses and blocked adenoviruses in Jurkat cells.

FIG. 20 shows the transduction efficiency of first-generation adenoviruses in primary, unactivated T cells as analyzed by FACS. Shown are results for three independently prepared primary T cell populations. Essentially no transduction could be seen in any of the experiments.

FIG. 21 shows the strategy employed in the parts of the current invention. Adenoviral vectors were constructed which display an anti-CD3 binding moiety, an anti-CD28 binding moiety and an interleukin 2 polypeptide on the knob of the viruses utilizing a combination of adapter molecules.

FIG. 22 shows an SDS-PAGE of the adapter molecules.

FIG. 23 shows that a combination of the adapters of the present disclosure leads to a strongly increased transduction efficiency as compared to adenoviruses displaying only one type of the adapters.

FIG. 24 compares the transduction efficiency of activated vs non-activated PBMC's. Cells were activated with Dynabeads (Human T-Expander CD3/CD28 (ThermoFisher)). Activation strongly increases transduction efficiency.

FIG. 25 demonstrates that the combination of the adapter molecules leads to a strongly increased transduction efficiency in T cells (Panel A), and that the combination of the adapter molecules leads to an activation of T cells (Panel B).

FIG. 26 shows the transduction efficiency of adenoviruses in T cells with various ratios of the adapter molecules.

FIG. 27 demonstrates that the combination of the adapter molecules not only work in first-generation adenoviruses, but also high capacity adenoviruses was equally high. In both types of adenoviruses, the transduction efficiencies for T cells are equally increased. GP/cell=genomic particles/cell.

FIG. 28 shows the oversaturation of the system as measured by a decrease in transduction efficiency. If the ratio of the adapter molecules to the number of adenoviruses increases, transduction efficiency decreases.

FIG. 29 demonstrates the CD3 specificity of the system. A mixture of wild type Jurkat cells and Jurkat cells with a CD3 knock-out were transfected with adapter molecules comprising an antigen-binding moiety specifically binding to CD3 (left panel) and with untargeted vectors (right panels). Only the cell population expressing CD3 (i.e., wild-type Jurkat cells) exhibited a strong increase in transduction efficiency when transduced with adapter molecules comprising an antigen-binding moiety specifically binding to CD3.

FIG. 30 shows the transduction frequency of PBMCs from three different donors. Cell populations with a CD3+ phenotype show a clearly increased transduction frequency when transduced with adenoviruses displaying the combination of vectors of the present disclosure.

FIG. 31 shows that transduction of PBMCs with adenoviruses displaying the combination of adapters of the present disclosure do not show a toxic overreaction. Cell numbers were similar to untransduced PBMCs.

FIG. 32 shows that transduction frequency of different cell populations isolated from NSG mice the were injected with human PBMCs and the adenoviruses displaying the combination of adapters of the present disclosure. T cells were by far the cell population that showed the highest transduction efficiency.

FIG. 33 shows in more details the efficiency of transduction of the T cell population shown in FIG. 32. It could be demonstrated that all T cell types, T helper cells and cytotoxic T cells, were transduced by adenoviruses displaying a combination of the adapters of the present disclosure. Left: all T cells; middle: T helper cells; right; cytotoxic T cells.

FIG. 34 shows transduction of unactivated T cells in NSG mice. Both types of T cells, T helper cells and cytotoxic T cells, could be transfected.

FIG. 35 shows transduction of non-stimulated T cells in reconstituted NSG mice. T cells (CD3+ phenotype) showed a significantly higher transduction efficiency compared to B cells (CD19+ phenotype).

FIG. 36 confirms the data shown in FIG. 35. Here, transduction of cells was quantified by qPCR.

FIG. 37 shows the transduction efficiency of adenoviruses in T cells with various ratios of the adapter molecules. In contrast to FIG. 8, T cells were pre-activated with dynabeads (Dynabeads Human T-Expander CD3/CD28 (ThermoFisher)).

### DEFINITIONS

The term “recombinant” as used in recombinant protein, recombinant protein domain and the like, means that said polypeptides are produced using recombinant DNA technologies well known by the practitioner skilled in the relevant art. For example, a recombinant DNA molecule (e.g. produced by gene synthesis) encoding a polypeptide can be cloned into a bacterial expression plasmid (e.g. pQE30, Qiagen) or a mammalian expression plasmid (e.g. pcDNA™3.1, ThermoFisher Scientific). When such a constructed recombinant expression plasmid is inserted into a host cell (e.g. E. coli, CHO, HEK), this host cell can produce the polypeptide encoded by this recombinant DNA. The correspondingly produced polypeptide is called a recombinant polypeptide.

The term “protein” as used herein refers to a polypeptide, wherein at least part of the polypeptide has, or can, acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulfide bond between two polypeptides.

A part of a protein, which individually has, or can acquire a defined three-dimensional arrangement by forming secondary or tertiary structures, is termed “protein domain” or “domain”. Such protein domains are well known to the practitioner skilled in the art.

The term “polypeptide” as used herein refers to a molecule consisting of one or more chains of multiple, i.e. two or more, amino acids linked via peptide bonds. A polypeptide typically consists of more than twenty amino acids linked via peptide bonds.

The term “peptide” as used herein refers to as used herein refers to a molecule consisting of one or more chains of multiple, i.e. two or more, amino acids linked via peptide bonds. A peptide typically consists of not more than twenty amino acids linked via peptide bonds.

The terms “designed ankyrin repeat protein”, “designed ankyrin repeat domain” and “DARPin” as used herein refer artificial polypeptides, comprising several ankyrin repeat motifs. These ankyrin repeat motifs provide a rigid interface arising from typically three repeated β-turns. DARPins usually carry two three repeats corresponding to an artificial consensus sequence, wherein six positions per repeat are randomized, flanked by two capping repeats with a hydrophilic surface (Curr Olpin Chem Biol (2009) 13:245-55; WO 02/20565).

The term “protein scaffold” means a protein with exposed surface areas in which amino acid insertions, substitutions or deletions are highly tolerable. Examples of protein scaffolds that can be used to generate binding domains of the present invention are antibodies or fragments thereof such as single-chain Fv or Fab fragments, T cell receptor such as single chain T cell receptors, protein A from Staphylococcus aureus, the bilin binding protein from Pieris brassicae or other lipocalins, ankyrin repeat proteins, monobodies, human fibronectin, or antibodies from camelids, such as nanobodies. Protein scaffolds are known to the person skilled in the art (Curr Opin Biotechnol 22:849-57 (2011); Ann Rev Pharmacol Toxicol 60:391-415 (2020)). In certain embodiments of the present disclosure the protein scaffold is a polypeptide. In certain embodiments of the present disclosure the protein scaffold is a monomeric polypeptide. In certain embodiments of the present disclosure, the protein scaffold is an antibody fragment. In certain embodiments of the present disclosure the protein scaffold is a scFv. In certain embodiments of the present disclosure the protein scaffold is a single chain T cell receptor. In certain embodiments of the present disclosure the protein scaffold is a peptide. In certain embodiments of the present disclosure the protein scaffold is not a designed ankyrin repeat domain.

The term “antibody” as used herein refers to a protein comprising at least two heavy (H) chains and two light (L) chains inter-connected by disulfide bonds, which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment” as used herein refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain variable fragment”, “single chain Fv” or “scFv”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1 126-1 136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The term “single chain TCR”, “single chain T cell receptor” and “scTCR” as used herein refers to any construct containing the variable domains of a T cell receptor in single chain format. Such single chain formats include, but are not limited to the constructs described in (J Immunol Methods (1998) 221:59-76, Cancer Immunol Immunother (2002) 51:565-73, WO 2019/219709 and WO 2004/033685. In certain embodiments single chain TCR variable domains and/or single chain TCRs may have a disulfide bonds as described in WO 2004/033685.

The term “immunoglobulin” as used herein refers to any polypeptide or fragment thereof from the class of polypeptides known to the skilled person under this designation and comprising at least one antigen binding site. Preferably, the immunoglobulin is a soluble immunoglobulin from any of the classes IgA, IgD, IgE, IgG, or IgM, or a fragment comprising at least one antigen binding site derived thereof. Also comprised as immunoglobulins of the present invention are a bispecific immunoglobulin, a synthetic immunoglobulin, an immunoglobulin fragment, such as Fab, Fv or scFv fragments etc., a single chain immunoglobulin, and a nanobody. Further included are chemically modified derivatives of any of the aforesaid, e.g. PEGylated derivatives, as well as fusion proteins comprising any of the aforesaid immunoglobulins and fragments thereof. The immunoglobulin may be a human or humanized immunoglobulin, a primatized, or a chimerized immunoglobulin or a fragment thereof as specified above. Preferably, the immunoglobulin of the present invention is a polyclonal or a monoclonal immunoglobulin, more preferably a monoclonal immunoglobulin or a fragment thereof as specified above.

The terms “binds”, “is specific” and “specifically binds” as used herein refers to a molecule, for example an antibody or an antibody fragment, which recognizes a specific antigen, but does not substantially recognize or bind other molecules in a sample. An antibody that specifically binds to an antigen from one species may also bind to that antigen from one or more further species. Such cross-species reactivity does not itself alter the classification of an antibody as specific.

The term “nucleic acid” as used herein refers to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases.

The term “vector” as used herein means a construct, which is capable of delivering, and usually expressing or regulating expression of, one or more gene(s) or nucleic acid(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “linker” as used herein refers a molecule or macromolecule serving to connect different moieties or domains of a peptide or a polypeptide or, a protein/polypeptide domain and a non-protein/non-polypeptide moiety. Linkers can be of different nature. Different domains or modules within proteins can be linked via peptide linkers. Linkers can also be generated chemically, for example to link small organic molecules or peptides to a protein.

The term “flexible linker” as used herein refers to a peptide linker linking two different domains or modules of a protein and providing a certain degree of flexibility. Preferably, the flexible linker is hydrophilic and does not interacting with other surfaces. Commonly used flexible linkers are glycine-serine linkers (Biochemistry 56 (50): 6565-6574 (2017)). Glycine and serine are flexible and the adjacent protein domains are free to move relative to one another. Such flexible linkers are referred to herein as “glycine-serine linkers”. Other amino acids commonly used in respective linkers are proline, asparagine and threonine. Often the linker contains several repeats of a sequence of amino acids. A flexible linker used in the present disclosure is a (Gly₄Ser)₄-linker, i.e. a linker containing four repeats of the sequence glycine-glycine-glycine-glycine-serine. Other linkers that could be used in accordance with the present disclosure include but are not limited to PAS linkers, i.e. linkers containing repeats of the sequence proline-alanine-serine (Protein Eng Des Sel (2013) 26, 489-501 and charged linkers.

The term “short linker” as used herein refers to a peptide linker linking two different domains or modules of a protein and which is no longer than four, preferably no longer than three amino acids long. More preferably the short linker is no longer than two amino acids long. Alternatively the short linker is only one amino acid long. Alternatively the short linker is a single glycine residue.

The term “amino acid mutation” refers to amino acid substitutions, deletions, insertions, and modifications, as well as combinations thereof. Amino acid sequence deletions and insertions include N- and/or C-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful.

The term “variant” as used herein refers to a polypeptide that differs from a reference polypeptide by one or more amino acid mutation or modifications.

The term “host cell” as used herein refers to any kind of cellular system which can be engineered to generate molecules according to the present disclosure. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Host cells according to the present disclosure can be a “eukaryotic host cell” and include yeast and mammalian cells, including murine cells and from other rodents, preferably vertebrate cells such as those from a mouse, rat, monkey or human cell line, for example HKB11 cells, PERC.6 cells, HEL293T cells, CHO cells or any type of HEK cells, such as HEK293 cells or HEK 993 cells. Also suspension cell lines like CHO-S or HEK993 cells, or insect cell cultures like Sf9 cells may be used.

Host cells according to the present disclosure can also be “procaryotic cell” and include bacterial cells, such Escherichia coli. Certain strains of Escherichia coli may be particularly useful for expression of the molecules of the present disclosure, such as Escherichia coli strain DH5 (available from Bethesda Research Laboratories, Inc., Bethesda, Md/US).

The molecules of the present disclosure form trimers that are highly stable. Each monomer contains a domain responsible for the formation of trimers which is referred to herein as “trimerization domain”. A preferred trimerization domain is the capsid protein SHP of lambdoid phage 21 (J Mol Biol; 344(1): 179-93; PNAS 110(10):E869-77 (2013)). SHP of lambdoid phage 21 has the following amino acid sequence:

(SEQ ID No. 1)
VRIFAGNDPAHTATGSSGISSPTPALTPLMLDEATGKLVVWDGQKAGSAV
GILVLPLEGTETALTYYKSGTFATEAIHWPESVDEHKKANAFAGSALSHA
ALP

The term “stable trimer” or “trimeric adapter” as used herein refers to a protein trimer by protein monomers comprising a trimerization domain, and wherein said trimer exhibits a stability which is higher than other, conventional protein trimers. For example, a stable trimer has a higher functional stability, a higher kinetic stability, or a higher high life for unfolding than other protein trimers. An example of a stable trimer is a trimer formed by monomers comprising the trimerization domain of the capsid protein SHP of lambdoid phage 21.

The term “derived from” in the context of an amino acid sequence refers to an amino acid sequence that is different to an original amino acid sequence, but maintains the function or activity of the original amino acid sequence.

The term “adenovirus” as used herein refers to any adenovirus, i.e. to human and non-human serotypes. The human isolates are classified into subgroups A-G. A preferred adenovirus of the present disclosure is adenovirus subtype 5 (“HAdV-C5”). HAdV-C5 includes modified version of the virus, such as replication-deficient HAdV-C5 version, e.g. containing an E1/E3 deletion and/or one or more of the 4 mutations in the HVR7 (1421G, T423N, E424S and L426Y) (Nat. Commun. 9, 450 (2018)).

The terms “CAR” and “CXADR” as used herein refers to coxsackievirus and adenovirus receptor (UniProt: P78310). CAR is a type I membrane receptor for coxsackie viruses and adenoviruses.

The term “knob” as used herein refers to a knob on the end of the adenovirus fiber (e.g. GenBank: AAP31231.1) that binds to the cellular receptor. The knob of adenovirus subtype 5 binds to CAR. Some adenoviruses carry mutations in the gene encoding the knob protein. Adenoviruses having a four-amino acid deletion within the FG loop of the knob (TAYT mutation) show a decreased ability of the mutated knob to bind to CAR (Science, 286:1568-1571 (1999); J Mol Biol 405 (2): 410-426). Adenoviruses carrying four amino acid mutations in the hypervariable region 7 (HVR7 mutation) show a strongly reduced binding to blood coagulation factor X (Nat Commun (2018) 9:450).

The molecules of the present invention contain a designed ankyrin repeat domain that binds to the knob of an adenovirus. A preferred designed ankyrin repeat domain that binds to a knob is DARPin 1D3. Another preferred designed ankyrin repeat domain that binds to a knob is DARPin 1D3nc, a derivative of 1D3nc containing a stabilized C-cap. DARPin 1D3 has the following amino acid sequence:

(SEQ ID No. 2)
RSDLGKKLLEAARAGQDDEVRILMANGADANAYDHYGRTPLHMAAAVGHL
EIVEVLLRNGADVNAVDTNGTTPLHLAASLGHLEIVEVLLKYGADVNAKD
ATGITPLYLAAYWGHLEIVEVLLKHGADVNAQDKFGKTPFDLAIDNGNED
IAEVLQG

The term “CD3” as used herein refers to human CD3 (cluster of differentiation 3), a protein complex and T cell co-receptor that involved in activating both the cytotoxic T cell (CD8+ naive T cells) and T helper cells (CD4+ naive T cells). It is composed of four distinct chains. The complex contains a CD3γ chain, a CD3δ chain, and two CD3ε chains.

An “antigen-binding moiety” as used herein refers to a polypeptide that specifically binds to an antigenic determinant. Exemplary antigen-binding moieties include, protein scaffold and antibodies and antibody fragment, such as a single-chain Fv or a Fab fragment.

An “antigen-binding moiety which specifically binds to CD3” refers to an antigen-binding moiety with binding specificity for CD3. In certain embodiments, the antigen-binding moiety which specifically binds to CD3 is a single-chain Fv. In certain embodiments, the antigen-binding moiety which specifically binds to CD3 comprises the following amino acid sequence:

(SEQ ID No. 11)
GPGSDIQMTQSPSSLSASVGDRVTITCRASQDIRNYLNWYQQKPGKAPKL
LIYYTSRLESGVPSRFSGSGSGTDYTLTISSLQPEDFATYYCQQGNTLPW
TFGQGTKVEIKRGGGGSGGGGSGGGGSEVQLVESGGGLVQPGGSLRLSCA
ASGYSFTGYTMNWVRQAPGKGLEWVALINPYKGVSTYNQKFKDRFTISVD
KSKNTAYLQMNSLRAEDTAVYYCARSGYYGDSDWYFDVWGQGTLVTVSSK
L.

In certain embodiments, the antigen-binding moiety which specifically binds to CD3 comprises an an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17).

The term “CD28” as used herein refers to human CD28 (UniProt: P10747), a protein essential for T-cell proliferation and survival, cytokine production, and T-helper type-2 development.

An “antigen-binding moiety which specifically binds to CD28” refers to an antigen-binding moiety with binding specificity for CD28. In certain embodiments, the antigen-binding moiety which specifically binds to CD28 is a single-chain Fv. In certain embodiments, the antigen-binding moiety which specifically binds to CD28 comprises the following amino acid sequence:

(SEQ ID No. 28)
GPGSDIQMTQSPSSLSASVGDRVTITCHASQNLYVWLNWYQQKPGKAPKL
LIYKASNLHTGVPSRFSGSGSGTDFTLTISSLQPEDFATYYCQQGQTYPY
TFGGGTKVEIKRGGGGSGGGGSGGGGSQVQLVQSGAEVKKPGASVKVSCK
ASGYTFTSYYIHWVRQAPGQGLEWIGCIYPGNVNTNYNEKFKDRATLTVD
TSISTAYMELSRLRSDDTAVYFCTRSHYGLDWNFDVWGQGTTVTVSSKL

In certain embodiments, the antigen-binding moiety which specifically binds to CD28 comprises an an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24).

The term “interleukin 2”, “IL2” or “IL-2” as used herein refers to human interleukin 2 (UniProt: P60568), a secreted cytokine produced by activated CD4+ and CD8+T lymphocytes, important for the proliferation of T and B lymphocytes. Human IL-2 has the following amino acid sequence:

(SEQ ID No. 3)
MYRMQLLSCIALSLALVINSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITECQSIIS
TLT

The term “functional interleukin 2” or “functional IL-2” as used herein refers to a polypeptide which is a variant or a derivative of human interleukin 2 which retains the biological function of wild type IL-2. Such variant or derivative may have one or more mutations compared to wild type IL-2. Such variant or derivative also may contain additions or deletions of amino acid sequences, in particular at the N-terminal or C-terminal part of IL-2. For example, the functional interleukin 2 may devoid of the signal sequence. In such embodiments, the functional interleukin 2 comprises the amino acid sequence:

(SEQ ID No. 4)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

The functional interleukin 2 may also comprise additional amino acids required for cloning. In certain embodiment the functional IL-2 polypeptide has the following amino acid sequence:

(SEQ ID No. 25)
GPGSAPTSSSTKKTQLQLEHLLLDLQMILNGINNYKNPKLTRMLTFKFYM
PKKATELKHLQCLEEELKPLEEVENLAQSKNFHLRPRDLISNINVIVLEL
KGSETTFMCEYADETATIVEFLNRWITFCQSIISTLTKL

The functional interleukin 2 may also be a variant of human IL-2 which retains the function of wild type human IL-2, such as a stability-enhanced variant of human IL-2. The functional interleukin 2 may also be a variant of human IL-2 with increase affinity to the IL-2 receptor a subunit. Exemplary variants of human IL-2 include those disclosed in Mol Pharmacol (2004) 66:864-9, U.S. Pat. No. 5,116,943, WO2005/086751, WO2012/062228, WO2012/088446, WO2014/153111 WO2019/246404, WO2020/020783, WO2020/125743, WO2020/187848, WO2020/201095, WO2020/252418, WO2020/252421, WO2020260270, WO2021/021606, WO2021146436 and WO2021185362.

Such variants also include the functional interleukin 2 variant which comprises the amino acid sequence:

(SEQ ID No. 5)
PTSSSTKKTQLQLEHLLLDLQMILNGINNYKNPKLTRMLTFKFYMPKKAT
ELKHLQCLEEELKPLEEVLNLAQSKNFHLRPRDLISNINVIVLELKGSET
TFMCEYADETATIVEFLNRWITFSQSIISTLT

The term “set” as used herein in the context of adenoviruses refers to a mixture of adenoviruses in which at least two adenoviruses are different. Such adenoviruses may for example contain a different genome, may have been transfected with different vectors or may display different adapter molecules.

The term “displaying” as used herein refers to the presentation of a polypeptide on the outside of an entity, such as an adenovirus. The polypeptides so presented on the entity may be covalently or non-covalently attached to such entity. In the context of the present disclosure adapter molecules are recombinantly expressed and displayed on adenoviruses. This can be accomplished via a binding moiety or a scaffold, such as a designed ankyrin repeat domain that binds to the knob of an adenovirus. Alternatively, moiety or scaffold can also be genetically fused to an adenoviral protein, such as the hexon.

The term “adapter” as used herein refers to a recombinant protein which comprises a designed ankyrin repeat domain which binds to the knob of the adenovirus, a trimerization domain and a domain which specifically binds to a target protein. The domain which specifically binds to a target protein that is employed in the present invention is a functional interleukin 2 polypeptide. Through this domain, the adapter links the adenovirus to target cells which express a receptor for the interleukin 2 polypeptide. Certain other adapters are use in the present disclosure. Such other adapters comprise different domains which specifically binds to a target protein, for example an anti-CD3 scFv or an anti-CD28 scFv.

The term “about the same time” as used herein refers to a time frame in which certain entities, for example immune cells, a recombinant adenovirus displaying a functional interleukin 2 polypeptide and an agent capable of activating immune cells, are mixed within a short time interval. This time interval is typically less than 10 minutes, preferably less than 5 minutes, and more preferably at the same time. If the entities are mixed at the same time this is referred to as “simultaneous” or “simultaneously”.

The term “agent capable of activating immune cells” as used herein refers to an agent that, when added to unactivated immune cells, such as T cells, activates such immune cells. Activated immune cells differ from unactivated immune cells by upregulation of markers such as CD25, CD69, PD-1, by a morphological change that encompasses an increase of diameter and increase of intracellular organelles and machinery, by an increased proliferation rate accompanied by doubling of cells up to every 6 hours. In the activated state such immune cells have an increase uptake or surrounding proteins and nutrients in response to the higher metabolic stress in preparation for expansion and cell division. Typical agents that are capable of activation immune cells are known to the skilled person and include DynaBeads (e.g. Dynabeads™ Human T-Expander CD3/CD28, Gibco, Catalogue number 11141D; Dynabeads™ Human T-Expander CD3/CD28/CD137, Gibco, Catalogue number 11162D), TransAct (T Cell TransAct™ human, Miltenyi, Catalogue number 130-111-160), Polybrene and PMA (1-Methoxy-2-propylacetat).

The term “extra corporeal” as used herein means refers to a procedure which is performed outside the body.

### EMBODIMENTS OF THE INVENTION

Disclosed herein is an adenoviral-based system for targeted transduction of immune cells, such as T cells. The system can be used in field of medicine, particularly in T cell-based or T cell-related diseases and disorders. Cargo, such as nucleic acids, in particular nucleic acids encoding therapeutically active or therapeutically helpful proteins and peptides, can be delivered into immune cells in which they can exert their function. One advantage of the adenoviral system utilized here is the fact that it can encode cargo of a large size, i.e. up to 36 kb.

Specificity of the adenoviruses can conferred by adapter molecules as described in the present disclosure. The specificity of the system and the correlating transduction efficiency is considerably higher than the systems known in the art. High transduction efficiencies can however be also obtained without adapter molecules. The system is functional with adenoviruses of any kind, i.e. first-generation virus, as well as high-capacity, helper virus-dependent adenoviral systems. The system is also functional with other viruses, e.g. viruses that are engineered to carry a knob of an adenovirus of subtype 5.

The methods provided herein, allow for the efficient transduction of immune cells. Said immune cells may be activated immune cells or non-activated immune cells.

In certain embodiments, the present disclosure relates to a recombinant adenovirus displaying functional interleukin 2 polypeptides. In certain embodiments the recombinant adenovirus display said functional interleukin 2 polypeptides via recombinant adapter molecules.

In certain embodiments, the present disclosure relates to a recombinant adenovirus a functional interleukin 2 polypeptide, wherein said adenovirus is capable of transducing immune cells. In other embodiments, the present disclosure relates to a recombinant adenovirus a functional interleukin 2 polypeptide, wherein said adenovirus is capable of transducing T cells. Human T cells lack strong expression of coxsackie and adenoviral receptors (CAR) through which transduction of adenoviruses is typically mediated, as well as RGD binding integrins which are involved in subsequent interactions. Adenoviruses armored with adapters as described in the present disclosure overcome these hurdles by generating a sufficiently high specificity to the T cells that enables adenoviral infection of the cells.

In certain embodiments of the present disclosure, the recombinant adenovirus comprises a functional interleukin 2 polypeptide. Many variants and derivatives of interleukin 2 are known in the art, all of which can be used in the context of the present disclosure, see e.g. Mol Pharmacol (2004) 66:864-9, U.S. Pat. No. 5,116,943, WO2005/086751, WO2012/062228, WO2012/088446, WO2014/153111 WO2019/246404, WO2020/020783, WO2020/125743, WO2020/187848, WO2020/201095, WO2020/252418, WO2020/252421, WO2020260270, WO2021/021606, WO2021146436 and WO2021185362. In certain embodiments, the functional interleukin 2 polypeptide comprises the amino acid sequence of SEQ ID No. 4. In certain embodiments, the functional interleukin 2 polypeptide consists of the amino acid sequence of SEQ ID No. 4. In certain embodiments, the functional interleukin 2 polypeptide comprises the amino acid sequence of SEQ ID No. 4 with one, two, three, four or five amino acid substitutions, insertions or deletions. In certain embodiments, the functional interleukin 2 polypeptide is a variant of the polypeptide consisting of the amino acid sequence of SEQ ID No. 4, wherein said variant is functionally equivalent to the polypeptide consisting of the amino acid sequence of SEQ ID No. 4. In certain embodiments, the functional interleukin 2 polypeptide comprises the amino acid sequence of SEQ ID No. 5. In certain embodiments, the functional interleukin 2 polypeptide consists of the amino acid sequence of SEQ ID No. 5. In certain embodiments, the functional interleukin 2 polypeptide comprises the amino acid sequence of SEQ ID No. 5 with one, two, three, four or five amino acid substitutions, insertions or deletions. In certain embodiments, the functional interleukin 2 polypeptide is a variant of the polypeptide consisting of the amino acid sequence of SEQ ID No. 5, wherein said variant is functionally equivalent to the polypeptide consisting of the amino acid sequence of SEQ ID No. 5.

In certain embodiments, the recombinant adenoviruses comprise recombinant polypeptides or proteins, that are displayed on said adenovirus. Said recombinant proteins comprises a functional interleukin 2 polypeptide. Said functional interleukin 2 polypeptide may be fused to the other parts of the recombinant protein in any order.

In certain embodiments, the present disclosure relates to a recombinant protein comprising
a) a designed ankyrin repeat domain which binds to the knob of an adenovirus,
b) a trimerization domain, and
c) a functional interleukin 2 polypeptide.

In certain embodiments, the present disclosure relates to a recombinant protein comprising from the N- to the C-terminus
a) a functional interleukin 2 polypeptide,
b) a designed ankyrin repeat domain which binds to the knob of an adenovirus, and
c) a trimerization domain.

In other embodiments, the present disclosure relates to a recombinant protein comprising from the N- to the C-terminus
a) a designed ankyrin repeat domain which binds to the knob of the adenovirus,
b) a trimerization domain, and
c) a functional interleukin 2 polypeptide.

In specific embodiments, the present disclosure relates to a recombinant protein comprising from the N- to the C-terminus
a) a functional interleukin 2 polypeptide,
b) a designed ankyrin repeat domain which binds to the knob of the adenovirus, and
c) a trimerization domain. In certain embodiments, the functional interleukin 2 polypeptide comprises the amino acid sequence of SEQ ID No. 4. In certain embodiments, the functional interleukin 2 polypeptide consists of the amino acid sequence of SEQ ID No. 4. In certain embodiments, the functional interleukin 2 polypeptide comprises the amino acid sequence of SEQ ID No. 4 with one, two, three, four or five amino acid substitutions, insertions or deletions. In certain embodiments, the functional interleukin 2 polypeptide is a variant of the polypeptide consisting of the amino acid sequence of SEQ ID No. 4, wherein said variant is functionally equivalent to the polypeptide consisting of the amino acid sequence of SEQ ID No. 4. In certain embodiments, the functional interleukin 2 polypeptide comprises the amino acid sequence of SEQ ID No. 5. In certain embodiments, the functional interleukin 2 polypeptide consists of the amino acid sequence of SEQ ID No. 5. In certain embodiments, the functional interleukin 2 polypeptide comprises the amino acid sequence of SEQ ID No. 5 with one, two, three, four or five amino acid substitutions, insertions or deletions. In certain embodiments, the functional interleukin 2 polypeptide is a variant of the polypeptide consisting of the amino acid sequence of SEQ ID No. 5, wherein said variant is functionally equivalent to the polypeptide consisting of the amino acid sequence of SEQ ID No. 5.

The recombinant proteins of the present disclosure comprise a designed ankyrin repeat domain which binds to the knob of a virus or adenovirus. It will be appreciated that any designed ankyrin repeat domain with specificity for the knob of a virus or adenovirus may be used within the spirit of the present disclosure. Exemplified herein is a designed ankyrin repeat domain derived from DARPin 1D3 (Proc. Natl. Acad. Sci. 110, E869-E877 (2013)). DARPin 1D3 binds to the knob of an adenovirus and comprises the amino acid sequence of SEQ ID No. 2. Used herein is 1D3nc, a derivative of 1D3 containing a stabilized C-cap.

It will also be understood that also variants of DARPin 1D3 may used within the spirit of the present disclosure. In other words, the amino acid sequence of such modified DARPin 1D3 does not need to be identical to that of amino acid sequence of SEQ ID No. 2, but may contain amino acids mutations, provided that the function of DARPin 1D3, i.e. binding to the knob of an adenovirus is preserved. Also DARPins different than 1D3, but having the same target specificity, may be used within the scope of the present disclosure. Such new DARPin may for example be selected in a new screening campaign. Also binding entities different than DARPins, i.e. binders based on a different scaffold, but having the same target specificity as 1D3 might be used.

Therefore, in certain embodiments, the present disclosure relates to a recombinant protein comprising
a) a designed ankyrin repeat domain which binds to the knob of an adenovirus,
b) a trimerization domain, and
c) a functional interleukin 2 polypeptide.

In certain embodiments, the present disclosure relates to a recombinant protein comprising
a) a designed ankyrin repeat domain which binds to the knob of an adenovirus,
b) a trimerization domain, and
c) a functional interleukin 2 polypeptide,
wherein said designed ankyrin repeat domain which binds to the knob of an adenovirus is DARPin 1D3.

In certain embodiments, the present disclosure relates to a recombinant protein comprising
a) a designed ankyrin repeat domain which binds to the knob of an adenovirus,
b) a trimerization domain, and
c) a functional interleukin 2 polypeptide,
wherein said designed ankyrin repeat domain which binds to the knob of an adenovirus is or is derived from DARPin 1D3.

In certain embodiments, the present disclosure relates to a recombinant protein comprising
a) a designed ankyrin repeat domain which binds to the knob of an adenovirus,
b) a trimerization domain, and
c) a functional interleukin 2 polypeptide,
wherein said designed ankyrin repeat domain which binds to the knob of an adenovirus is a variant of DARPin 1D3.

In certain embodiments, the present disclosure relates to a recombinant protein comprising
a) a designed ankyrin repeat domain which binds to the knob of an adenovirus,
b) a trimerization domain, and
c) a functional interleukin 2 polypeptide,
wherein said designed ankyrin repeat domain which binds to the knob of an adenovirus comprises the amino acid sequence of SEQ ID No. 2.

In certain embodiments, the present disclosure relates to a recombinant protein comprising
a) a designed ankyrin repeat domain which binds to the knob of an adenovirus,
b) a trimerization domain, and
c) a functional interleukin 2 polypeptide,
wherein said designed ankyrin repeat domain which binds to the knob of an adenovirus comprising a variant of the amino acid sequence of SEQ ID No. 2.

In certain embodiments, the present disclosure relates to recombinant proteins comprising a designed ankyrin repeat domain that binds to the knob of an adenovirus. The present disclosure can however also be practiced with other viruses. If another virus is used a designed ankyrin repeat domain needs to be selected that binds to the knob of such virus. Therefore, in certain embodiments the present disclosure relates to recombinant proteins comprising
a) a designed ankyrin repeat domain which binds to the knob of the adenovirus,
b) trimerization domain, and
c) a functional interleukin 2 polypeptide.

Viruses, other than adenoviruses of serotype 5, can also be used within the spirit of the present disclosure. For example, such viruses can be engineered to carry the knob of an adenovirus of serotype 5. The recombinant protein disclosed herein, in particular recombinant protein comprising DARPin 1D3, may then be used with such viruses. Therefore, in certain embodiments the present disclosure provides a recombinant virus or a set of recombinant viruses comprising a recombinant protein comprising:
a) a functional interleukin 2 polypeptide,
b) a designed ankyrin repeat domain which binds to the knob of an adenovirus, and
c) a trimerization domain,
   wherein said designed ankyrin repeat domain binds to the knob of an adenovirus of subtype 5.

It will be understood that also other adenoviral serotypes may be used in the spirit of the present disclosure, including human adenovirus serotype c5 (HAdV-C5), HAd2, HAd3, HAdV-B35, HAdV-D26, as well as hybrids thereof. A list of adenoviruses can be found on the website of the Human Adenovirus Working group (http://hadvwg.gmu.edu). Also, non-human adenoviruses may be used within the scope of the present disclosure, such as the AstraZeneca vaccine chimpanzee adenovirus Y25 (CHAdY25), or non-human adenoviral vectors were developed from bovine (BAd), canine (CAd), chimpanzee (ChAd), ovine (OAd), porcine (PAd), or fowl (FAd).

A preferred virus to be used in the context of the present disclosure is adenovirus of serotype 5. Therefore, in certain embodiments the present disclosure relates to recombinant proteins comprising
a) a designed ankyrin repeat domain which binds to the knob of an adenovirus,
b) a trimerization domain, and
c) a functional interleukin 2 polypeptide,
wherein said designed ankyrin repeat domain binds to the knob of an adenovirus of serotype 5.

In certain embodiments, the present disclosure relates to recombinant proteins or uses of such recombinant proteins that comprise a trimerization domain. The trimerization domain is responsible for the formation of trimers. Each monomer of the molecules of the present disclosure comprises a trimerization domain. Principally any trimerization domain may be used, provided it is stable and geometrically fits the knob of the adenovirus used. A preferred trimerization domain is the capsid protein SHP of lambdoid phage 21 (J Mol Biol; 344 (1): 179-93; PNAS 110 (10): E869-77 (2013)).

Therefore, in certain embodiments the present disclosure relates to recombinant proteins comprising
a) a designed ankyrin repeat domain which binds to the knob of an adenovirus,
b) a trimerization domain, and
c) a functional interleukin 2 polypeptide,
wherein said trimerization domain is the capsid protein SHP of lambdoid phage 21.

In other embodiments the present disclosure relates to recombinant proteins comprising
a) a designed ankyrin repeat domain which binds to the knob of an adenovirus,
b) a trimerization domain, and
c) a functional interleukin 2 polypeptide,
wherein said trimerization domain is derived from the capsid protein SHP of lambdoid phage 21.

In other embodiments the present disclosure relates to recombinant proteins comprising
a) a designed ankyrin repeat domain which binds to the knob of an adenovirus,
b) a trimerization domain, and
c) a functional interleukin 2 polypeptide,
wherein said trimerization domain comprises the amino acid sequence of SEQ ID No. 1.

Also, other trimerization domains known to the skilled person may be used for the formation or trimers. Without being limited other potential trimerization domains include the trimerization domain involved in collagen folding (Int J Biochem Cell Biol 44:21-32 (2012)), the trimerization domain of T4 phage fibritin (PLOS One 7: e43603 (2012)) or the GCN4-based isoleucine zipper (J Biol Chem 290:7436-42 (2015)).

The trimerization domain is responsible for the formation of the trimeric adapter molecules. The trimers disclosed herein are extraordinary stable (J Mol Biol (2004) 344:179-93; PNAS (2013) 110 E869-77). In certain embodiments the trimeric adapter molecules of the present disclosure remain intact in SDS gel electrophoresis. In other embodiments the trimeric adapter molecules are not denatured in SDS gel electrophoresis. In other embodiments the trimeric adapter molecules have a half-life in solution of at least one week, preferably at least two week and even more preferably at least one month.

Therefore, in certain embodiments the present disclosure relates to recombinant proteins comprising
a) a designed ankyrin repeat domain which binds to the knob of an adenovirus,
b) a trimerization domain, and
c) a functional interleukin 2 polypeptide,
wherein said trimerization domain has a half-life in solution of at least one week, preferably at least two week and even more preferably at least one month.

The recombinant proteins of the present disclosure are encoded by nucleic acids. Vectors comprising these nucleic acids are used to transfect cells which express the recombinant proteins.

Therefore, in certain embodiments, the present disclosure relates to a nucleic acid encoding a recombinant protein of the present disclosure. The present disclosure also relates to a nucleic acid encoding a recombinant protein comprising
a) a designed ankyrin repeat domain which binds to the knob of an adenovirus,
b) a trimerization domain, and
c) a functional interleukin 2 polypeptide.

In other embodiments, the present disclosure relates to a vector comprising a nucleic acid encoding a recombinant protein of the present disclosure. The present disclosure also relates to a vector comprising a nucleic acid encoding a recombinant protein comprising
a) a designed ankyrin repeat domain which binds to the knob of an adenovirus,
b) a trimerization domain, and
c) a functional interleukin 2 polypeptide.

In another embodiments, the present disclosure relates to an adenovirus comprising a nucleic acid encoding a recombinant protein of the present disclosure. In yet another embodiment, the present disclosure relates to an adenovirus comprising a vector comprising a nucleic acid encoding a recombinant protein of the present disclosure. In certain embodiments said adenovirus carries a TAYT mutation. In certain embodiments said adenovirus carries a HVR7 mutation.

In certain embodiments, the present disclosure also relates to an adenoviral vector comprising a nucleic acid encoding a recombinant protein comprising
a) a designed ankyrin repeat domain which binds to the knob of an adenovirus,
b) a trimerization domain, and
c) a functional interleukin 2 polypeptide.

The recombinant protein of the present disclosure may also comprise a flexible linker. If the recombinant protein comprising from the N- to the C-terminus
a) a functional interleukin 2 polypeptide,
b) a designed ankyrin repeat domain which binds to the knob of an adenovirus, and
c) a trimerization domain,
then said flexible linker is between said functional interleukin 2 polypeptide and said designed ankyrin repeat domain which binds to the knob of an adenovirus.

Therefore, in certain embodiments the present disclosure relates to a recombinant protein comprising from the N- to the C-terminus
a) a functional interleukin 2 polypeptide,
b) a flexible linker,
c) a designed ankyrin repeat domain which binds to the knob of an adenovirus, and
d) a trimerization domain.

If the recombinant protein comprising from the N- to the C-terminus
a) a designed ankyrin repeat domain which binds to the knob of the adenovirus,
b) a trimerization domain, and
c) a functional interleukin 2 polypeptide,
then said flexible linker is between said trimerization domain and said functional interleukin 2 polypeptide.

Therefore, in certain embodiments the present disclosure relates to a recombinant protein comprising from the N- to the C-terminus
a) a designed ankyrin repeat domain which binds to the knob of the adenovirus,
b) a trimerization domain,
c) a flexible linker, and
d) a functional interleukin 2 polypeptide.

Principally any flexible linker can be used within the spirit of the present disclosure. Certain preferred flexible linkers are glycine-serine linkers. A particularly preferred flexible linker is a (Gly₄Ser)₄-linker.

Therefore, in certain embodiments the present disclosure relates to a recombinant protein comprising from the N- to the C-terminus
a) a functional interleukin 2 polypeptide,
b) a flexible linker,
c) a designed ankyrin repeat domain which binds to the knob of an adenovirus, and
d) a trimerization domain,
wherein said flexible linker is a glycine-serine linker.

In other embodiments the present disclosure relates to a recombinant protein comprising from the N- to the C-terminus
a) a functional interleukin 2 polypeptide,
b) a flexible linker,
c) a designed ankyrin repeat domain which binds to the knob of an adenovirus, and
d) a trimerization domain,
wherein said flexible linker is a (Gly₄Ser)₄-linker.

The recombinant protein of the present disclosure may also comprise a short linker. The short linker is located between the designed ankyrin repeat domain which binds to the knob of an adenovirus and the trimerization domain.

Therefore, in certain embodiments the present disclosure relates to a recombinant protein comprising from the N- to the C-terminus
a) a functional interleukin 2 polypeptide,
b) a designed ankyrin repeat domain which binds to the knob of an adenovirus,
c) a short linker, and
d) a trimerization domain.

In other embodiments the present disclosure relates to a recombinant protein comprising from the N- to the C-terminus
a) a designed ankyrin repeat domain which binds to the knob of the adenovirus,
b) a short linker,
c) a trimerization domain, and
d) a functional interleukin 2 polypeptide.

The short linker does not necessarily be present. Possible short linkers of the present disclosure are linkers which are no longer than four, no longer than three, no longer than two or only one amino acid long. A preferred short linker is glycine. Another preferred short linker is glycine-alanine. Most preferably the short linker is absent.

In certain embodiments, the present disclosure relates to a trimeric protein consisting of three recombinant proteins described herein above.

The recombinant proteins of the present disclosure can be expressed in prokaryotic cells, such as Escherichia coli, and in eukaryotic cells. Preferred eukaryotic cells are CHO cells. Other preferred eukaryotic cells are HEK293 cells, HEK293-T cells, HEK293-F cells, CHO-S cells and Sf9 cells. Therefore, in certain embodiments the present disclosure provides a eukaryotic cell expressing the recombinant protein of the present disclosure. In certain other the present disclosure provides a CHO cell expressing the recombinant protein of the present disclosure. In certain embodiments, the present disclosure relates to a eukaryotic cell expressing a recombinant protein comprising
a) a functional interleukin 2 polypeptide,
b) a designed ankyrin repeat domain which binds to the knob of the adenovirus, and
c) a trimerization domain.

In certain embodiments, the present disclosure relates to a CHO cell expressing a recombinant protein comprising
a) a functional interleukin 2 polypeptide,
b) a designed ankyrin repeat domain which binds to the knob of the adenovirus, and
c) a trimerization domain.

Aforementioned recombinant adenoviruses displaying a functional interleukin 2 polypeptide are used for the transduction of immune cells. Therefore, in certain embodiments the present disclosure relates to a method for the transduction of immune cells, said method comprising
a. contacting said immune cells with
   i. a recombinant adenovirus displaying a functional interleukin 2 polypeptide, and
   ii. an agent capable of activating said immune cells, and
b. incubating the mixture obtained in step a. for a time sufficient for transduction of said immune cells.

In other embodiments, the present disclosure relates to a method for the transduction of immune cells, said method comprising
a. contacting said immune cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said immune cells.

In other embodiments, the present disclosure relates to a method for the transduction of immune cells, said method comprising
a. contacting said immune cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said immune cells.

In other embodiments, the present disclosure relates to a method for the transduction of immune cells, said method comprising
a. contacting said immune cells at about the same time with
   i. a recombinant adenovirus, and
   ii. an agent capable of activating said immune cells, and
b. incubating the mixture obtained in step a. for a time sufficient for transduction of said immune cells.

In other embodiments, the present disclosure relates to a method for the transduction of immune cells, said method comprising
a. contacting said immune cells simultaneously with
   i. a recombinant adenovirus, and
   ii. an agent capable of activating said immune cells, and
b. incubating the mixture obtained in step a. for a time sufficient for transduction of said immune cells.

The method disclosed herein can be employed used for the transduction of any immune cells. Such immune cells include T cells and NK cells. Said T cells may be CD4-positve T cells or CD8-positive T cells. Said immune cells may also be CD4-positve T cells which are CD25^{high} and CD127^{low}, cells known as T regulatory cell (Treg cells).

Therefore, in certain embodiments, the present disclosure relates to a method for the transduction of T cells or NK cells, said method comprising
a. contacting said T cells or NK cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said immune cells.

In other embodiments, the present disclosure relates to a method for the transduction of T cells, said method comprising
a. contacting said T cells at about the same time with
   i. a recombinant adenovirus displaying a functional interleukin 2 polypeptide, and
   ii. an agent capable of activating said immune cells, and b. incubating the mixture obtained in step a. for a time sufficient for transduction of said T cells.

In other embodiments, the present disclosure relates to a method for the transduction of T cells, said method comprising
a. contacting said T cells at about the same time with
   i. a recombinant adenovirus, and
   ii. an agent capable of activating said immune cells, and
b. incubating the mixture obtained in step a. for a time sufficient for transduction of said T cells.

In other embodiments, the present disclosure relates to a method for the transduction of CD4-positive T cells, said method comprising
a. contacting said CD4-positive T cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said CD4-positive T cells.

In other embodiments, the present disclosure relates to a method for the transduction of CD4-positive T cells, said method comprising
a. contacting said CD4-positive T cells at about the same time with
   i. a recombinant adenovirus, and
   ii. an agent capable of activating said immune cells, and
b. incubating the mixture obtained in step a. for a time sufficient for transduction of said CD4-positive T cells.

In other embodiments, the present disclosure relates to a method for the transduction of CD8-positive T cells, said method comprising
a. contacting said CD8-positive T cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said CD8-positive T cells.

In other embodiments, the present disclosure relates to a method for the transduction of CD8-positive T cells, said method comprising
a. contacting said CD8-positive T cells at about the same time with
   i. a recombinant adenovirus, and
   ii. an agent capable of activating said immune cells, and
b. incubating the mixture obtained in step a. for a time sufficient for transduction of said CD8-positive T cells.

In other embodiments, the present disclosure relates to a method for the transduction of Treg cells, said method comprising
a. contacting said Treg cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said Treg cells.

In other embodiments, the present disclosure relates to a method for the transduction of Treg cells, said method comprising
a. contacting said Treg cells at about the same time with
   i. a recombinant adenovirus, and
   ii. an agent capable of activating said immune cells, and
b. incubating the mixture obtained in step a. for a time sufficient for transduction of said Treg cells.

In other embodiments, the present disclosure relates to a method for the transduction of NK cells, said method comprising
a. contacting said NK cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said NK cells.

In other embodiments, the present disclosure relates to a method for the transduction of NK cells, said method comprising
a. contacting said NK cells at about the same time with
   i. a recombinant adenovirus, and
   ii. an agent capable of activating said immune cells, and
b. incubating the mixture obtained in step a. for a time sufficient for transduction of said NK cells.

In the method of the present disclosure, immune cells are activated at about the same time or simultaneously to the transduction with a recombinant adenovirus displaying a functional interleukin 2 polypeptide. Said activation is brought about by an agent capable of activating said immune cells. Such agents include but are not limited to e.g. DynaBeads (e.g. Dynabeads™ Human T-Expander CD3/CD28, Gibco, Catalogue number 11141D; Dynabeads™ Human T-Expander CD3/CD28/CD137, Gibco, Catalogue number 11162D), TransAct (T Cell TransAct™ human, Miltenyi, Catalogue number 130-111-160), Polybrene and PMA (1-Methoxy-2-propylacetat). These substances lead to an activation of several immune cells.

Therefore, in certain embodiments, the present disclosure relates to a method for the transduction of immune cells, said method comprising
a. contacting said immune cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said immune cells,
wherein said agent capable of activating said immune cells is DynaBeads.

In certain embodiments, the present disclosure relates to a method for the transduction of immune cells, said method comprising
a. contacting said immune cells at about the same time with
   i. a recombinant adenovirus, and
   ii. an agent capable of activating said immune cells, and
b. incubating the mixture obtained in step a. for a time sufficient for transduction of said immune cells,
wherein said agent capable of activating said immune cells is DynaBeads.

In other embodiments, the present disclosure relates to a method for the transduction of immune cells, said method comprising
a. contacting said immune cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said immune cells,
wherein said agent capable of activating said immune cells is TransAct.

In other embodiments, the present disclosure relates to a method for the transduction of immune cells, said method comprising
a. contacting said immune cells simultaneously with
   i. a recombinant adenovirus, and
   ii. an agent capable of activating said immune cells, and
b. incubating the mixture obtained in step a. for a time sufficient for transduction of said immune cells,
wherein said agent capable of activating said immune cells is TransAct. In other embodiments, the present disclosure relates to a method for the transduction of immune cells, said method comprising
a. contacting said immune cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said immune cells,
wherein said agent capable of activating said immune cells is Polybrene.

In other embodiments, the present disclosure relates to a method for the transduction of immune cells, said method comprising
a. contacting said immune cells simultaneously with
   i. a recombinant adenovirus, and
   ii. an agent capable of activating said immune cells, and
b. incubating the mixture obtained in step a. for a time sufficient for transduction of said immune cells,
wherein said agent capable of activating said immune cells is Polybrene.

In other embodiments, the present disclosure relates to a method for the transduction of immune cells, said method comprising
a. contacting said immune cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said immune cells,
wherein said agent capable of activating said immune cells is PMA.

In other embodiments, the present disclosure relates to a method for the transduction of immune cells, said method comprising
a. contacting said immune cells simultaneously with
   i. a recombinant adenovirus, and
   ii. an agent capable of activating said immune cells, and
b. incubating the mixture obtained in step a. for a time sufficient for transduction of said immune cells,
wherein said agent capable of activating said immune cells is PMA.

The method disclosed herein above can be performed in vivo or in vitro. In preferred embodiments said method is performed in vitro.

Therefore, in certain embodiments, the present disclosure relates to an in vitro method for the transduction of immune cells, said method comprising
a. contacting said immune cells at about the same time with
   i. a recombinant adenovirus displaying a functional interleukin 2 polypeptide, and
   ii. an agent capable of activating said immune cells, and
b. incubating the mixture obtained in step a. for a time sufficient for transduction of said immune cells.

In certain embodiments, the present disclosure relates to an in vitro method for the transduction of immune cells, said method comprising
a. contacting said immune cells at about the same time with
   i. a recombinant adenovirus, and
   ii. an agent capable of activating said immune cells, and
b. incubating the mixture obtained in step a. for a time sufficient for transduction of said immune cells.

In other embodiments, the present disclosure relates to an in vitro method for the transduction of immune cells, said method comprising
a. contacting said immune cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said immune cells.

In other embodiments, the present disclosure relates to an in vitro method for the transduction of immune cells, said method comprising
a. contacting said immune cells simultaneously with
   i. a recombinant adenovirus, and
   ii. an agent capable of activating said immune cells, and
b. incubating the mixture obtained in step a. for a time sufficient for transduction of said immune cells.

In other embodiments, the present disclosure relates to an in vitro method for the transduction of T cells, said method comprising
a. contacting said T cells at about the same time with
   i. a recombinant adenovirus displaying a functional interleukin 2 polypeptide, and
   ii. an agent capable of activating said T cells, and
b. incubating the mixture obtained in step a. for a time sufficient for transduction of said T cells.

In other embodiments, the present disclosure relates to an in vitro method for the transduction of T cells, said method comprising
a. contacting said T cells at about the same time with
   i. a recombinant adenovirus, and
   ii. an agent capable of activating said T cells, and
b. incubating the mixture obtained in step a. for a time sufficient for transduction of said T cells.

In other embodiments, the present disclosure relates to an in vitro method for the transduction of T cells, said method comprising
a. contacting said T cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said T cells.

In other embodiments, the present disclosure relates to an in vitro method for the transduction of T cells, said method comprising
a. contacting said T cells simultaneously with
   i. a recombinant adenovirus, and
   ii. an agent capable of activating said immune cells, and
b. incubating the mixture obtained in step a. for a time sufficient for transduction of said T cells.

In other embodiments, the present disclosure relates to an in vitro method for the transduction of NK cells, said method comprising
a. contacting said NK cells at about the same time with
   i. a recombinant adenovirus displaying a functional interleukin 2 polypeptide, and
   ii. an agent capable of activating said T cells, and
b. incubating the mixture obtained in step a. for a time sufficient for transduction of said T cells.

In other embodiments, the present disclosure relates to an in vitro method for the transduction of NK cells, said method comprising
a. contacting said NK cells at about the same time with
   i. a recombinant adenovirus, and
   ii. an agent capable of activating said T cells, and
b. incubating the mixture obtained in step a. for a time sufficient for transduction of said T cells.

In other embodiments, the present disclosure relates to an in vitro method for the transduction of NK cells, said method comprising
a. contacting said T cells simultaneously with
   i. a recombinant adenovirus displaying a functional interleukin 2 polypeptide, and
   ii. an agent capable of activating said immune cells, and
b. incubating the mixture obtained in step a. for a time sufficient for transduction of said NK cells.

In other embodiments, the present disclosure relates to an in vitro method for the transduction of NK cells, said method comprising
a. contacting said T cells simultaneously with
   i. a recombinant adenovirus, and
   ii. an agent capable of activating said immune cells, and
b. incubating the mixture obtained in step a. for a time sufficient for transduction of said NK cells.

The recombinant proteins of the present disclosure, the nucleic acids encoding the recombinant proteins and trimeric proteins of the present disclosure, the vectors containing the nucleic acids of the present disclosure and the recombinant adenoviruses containing the recombinant proteins, the nucleic acids or the vectors of the present disclosure have numerous uses, such as the use in an adenoviral delivery system. Therefore, in certain embodiments the present disclosure provides the use of the recombinant proteins of the present disclosure in an adenoviral delivery system. In other embodiments the present disclosure provides the use of the nucleic acids encoding the recombinant proteins of present disclosure in an adenoviral delivery system. In other embodiments the present disclosure provides the use of the vectors containing the nucleic acids of the present disclosure in an adenoviral delivery system. In other embodiments the present disclosure provides the use of the adenoviruses containing the recombinant proteins, the nucleic acids or the vectors of the present disclosure in an adenoviral delivery system. Preferably, in said adenoviral delivery system immune cell, such as T cells or NK cells, are contacted about the same time with a recombinant adenovirus displaying a functional interleukin 2 polypeptide and an agent capable of activating said T cells or NK cells. More preferably, said immune cell, such as T cells or NK cells, are contacted simultaneously with a recombinant adenovirus displaying a functional interleukin 2 polypeptide and an agent capable of activating said T cells or NK cells.

The recombinant proteins of the present disclosure, the nucleic acids encoding the recombinant proteins and trimeric proteins of the present disclosure, the vectors containing the nucleic acids of the present disclosure and the recombinant adenoviruses containing the recombinant proteins, the nucleic acids or the vectors of the present disclosure can be used for the transduction of immune cells. Therefore, in certain embodiments the present disclosure provides the use of the recombinant proteins of the present disclosure for the transduction of immune cells. Preferably said immune cells are T cells or NK cells. Therefore, in certain embodiments the present disclosure provides the use of the recombinant proteins of the present disclosure for the transduction of T cells or NK cells. In other embodiments the present disclosure provides the use of the nucleic acids encoding the recombinant proteins of present disclosure for the transduction of T cells or NK cells. In other embodiments the present disclosure provides the use of the vectors containing the nucleic acids of the present disclosure for the transduction of T cells or NK cells. In other embodiments the present disclosure provides the use of the adenoviruses containing the recombinant proteins, the nucleic acids or the vectors of the present disclosure for the transduction of T cells or NK cells. Preferably, in said adenoviral delivery system immune cell, such as T cells or NK cells, are contacted about the same time with a recombinant adenovirus displaying a functional interleukin 2 polypeptide and an agent capable of activating said T cells or NK cells. More preferably, said immune cell, such as T cells or NK cells, are contacted simultaneously with a recombinant adenovirus displaying a functional interleukin 2 polypeptide and an agent capable of activating said T cells or NK cells.

The recombinant proteins of the present disclosure, the nucleic acids encoding the recombinant proteins and trimeric proteins of the present disclosure, the vectors containing the nucleic acids of the present disclosure and the recombinant adenoviruses containing the recombinant proteins, the nucleic acids or the vectors of the present disclosure can also be used in medicine. Therefore, in certain embodiments the present disclosure provides the use of the recombinant proteins of the present disclosure in medicine. In other embodiments the present disclosure provides the use of the nucleic acids encoding the recombinant proteins of the present disclosure in medicine. In other embodiments the present disclosure provides the use of the vectors containing the nucleic acids of the present disclosure in medicine. In other embodiments the present disclosure provides the use of the adenoviruses containing the recombinant proteins, the nucleic acids or the vectors of the present disclosure in medicine. Preferably, in said use in medicine immune cell, such as T cells or NK cells, are contacted about the same time with a recombinant adenovirus displaying a functional interleukin 2 polypeptide and an agent capable of activating said T cells or NK cells. More preferably, said immune cell, such as T cells or NK cells, are contacted simultaneously with a recombinant adenovirus displaying a functional interleukin 2 polypeptide and an agent capable of activating said T cells or NK cells.

In certain embodiments the present disclosure provides a method to treat a patient, said method comprising administering to a patient in need of a recombinant protein of the present disclosure. In certain embodiments the present disclosure provides a method to treat a patient, said method comprising administering to a patient in need of a nucleic acid encoding a recombinant protein of the present disclosure. In certain embodiments the present disclosure provides a method to treat a patient, said method comprising administering to a patient in need thereof a vector containing a nucleic acid of the present disclosure. In certain embodiments the present disclosure provides a method to treat a patient, said method comprising administering to a patient in need thereof a recombinant adenovirus containing a recombinant protein, a nucleic acid or a vector of the present disclosure. Preferably, in said method to treat a patient immune cell, such as T cells, are contacted about the same time with a recombinant adenovirus displaying a functional interleukin 2 polypeptide and an agent capable of activating said T cells or NK cells. More preferably, said immune cell, such as T cells or NK cells, are contacted simultaneously with a recombinant adenovirus displaying a functional interleukin 2 polypeptide and an agent capable of activating said T cells or NK cells.

The recombinant proteins of the present disclosure, the nucleic acids encoding the recombinant proteins of the present disclosure, the vectors containing the nucleic acids of the present disclosure, the recombinant adenovirus or set of recombinant adenoviruses displaying or containing the recombinant proteins, the nucleic acids or the vectors of the present disclosure, and the eukaryotic cells containing the recombinant adenovirus or set of recombinant adenoviruses of the present disclosure can be used in the treatment or prevention of any disease or disorder.

The present disclosure also relates to a recombinant adenovirus or a set of recombinant adenoviruses displaying molecules that are specific for antigen expressed on T cells. In certain embodiments the recombinant adenovirus or set of recombinant adenoviruses display a first antigen-binding moiety with specificity for an antigen expressed on T cells and a second antigen-binding moiety with specificity for an antigen expressed on T cells. In certain embodiments the antigen bound by the first antigen-binding moiety and the antigen bound by the second antigen-binding moiety are identical. In certain embodiments the antigen bound by the first antigen-binding moiety and the antigen bound by the second antigen-binding moiety are identical, but the first antigen-binding moiety binds to a different epitope on the antigen than the second antigen-binding moiety. In certain embodiments the antigen bound by the first antigen-binding moiety and the antigen bound by the second antigen-binding moiety are different. The antigen bound by the antigen-binding moiety can be any antigen that is specific for T cells (helper, effector, regulatory or γδ T cells). In certain embodiments the T-cell-specific antigen is CD28. In other embodiments the T-cell-specific antigen is CD3. In certain embodiments the first T-cell-specific antigen is CD28 and the second T-cell-specific antigen is CD3.

In certain embodiments the recombinant adenovirus or set of recombinant adenoviruses display an antigen-binding moiety with specificity for an antigen expressed on T cells and a component which is involved in the activation of T cells, such as interleukin 2. In certain embodiments the T-cell-specific antigen is CD28 and the component which is involved in the activation of T cells is interleukin 2. In other embodiments the T-cell-specific antigen is CD3 and the component which is involved in the activation of T cells is interleukin 2.

In certain embodiments the recombinant adenovirus or set of recombinant adenoviruses display a first antigen-binding moiety with specificity for an antigen expressed on T cells, a second first antigen-binding moiety with specificity for an antigen expressed on T cells, and a component which is involved in the activation of T cells. In certain embodiments the first T-cell-specific antigen is CD28, the second T-cell-specific antigen is CD3, and the component which is involved in the activation of T cells is interleukin 2.

In certain embodiments, the present disclosure relates to a recombinant adenovirus or a set of recombinant adenoviruses displaying
a) an antigen-binding moiety specifically binding to CD3, and/or
b) an antigen-binding moiety specifically binding to CD28, and/or
c) a functional interleukin 2 polypeptide.

In certain embodiments of the present disclosure, the recombinant adenovirus or the set of recombinant adenoviruses are capable of transducing T cells. Human T cells poorly express coxsackie and adenoviral receptors (CAR) through which transduction of adenoviruses is typically mediated, as well as RGD binding integrins which are involved in subsequent interactions. Adenoviruses armored with adapters as described in the present disclosure overcome these hurdles by generating a sufficiently high specificity to the T cells that enables adenoviral infection of the cells.

In certain embodiments of the present disclosure, the recombinant adenovirus or the set of recombinant adenoviruses comprise an antigen-binding moiety specifically binding to CD3. In certain embodiments, said antigen-binding moiety specifically binding to CD3 is a single-chain antibody. In certain embodiments, said antigen-binding moiety specifically binding to CD3 comprises an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17). In certain embodiments, said antigen-binding moiety specifically binding to CD3 comprises an amino acid sequence of SEQ ID No. 11. In certain embodiments, said antigen-binding moiety specifically binding to CD3 comprises an amino acid sequence with at least 90%, preferably at least 95% and more preferably at least 98% homology to the amino acid sequence of SEQ ID No. 11.

In certain embodiments, said antigen-binding moiety specifically binding to CD3 competes for binding to CD3 with an antigen-binding moiety comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17). In certain embodiments, said antigen-binding moiety specifically binding to CD3 is a single chain antibody. In certain embodiments, said antigen-binding moiety specifically binding to CD3 competes for binding to CD3 with an antigen-binding moiety comprising an amino acid sequence of SEQ ID No. 11.

In certain embodiments of the present disclosure, the recombinant adenovirus or the set of recombinant adenoviruses comprise an antigen-binding moiety specifically binding to CD28. In certain embodiments, said antigen-binding moiety specifically binding to CD28 is a single-chain antibody. In certain embodiments, said antigen-binding moiety specifically binding to CD28 an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24). In certain embodiments, said antigen-binding moiety specifically binding to CD28 comprises an amino acid sequence of SEQ ID No. 28. In certain embodiments, said antigen-binding moiety specifically binding to CD28 comprises an amino acid sequence with at least 90%, preferably at least 95% and more preferably at least 98% homology to the amino acid sequence of SEQ ID No. 28.

In certain embodiments, said antigen-binding moiety specifically binding to CD28 competes for binding to CD28 with an antigen-binding moiety comprising an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24). In certain embodiments, said antigen-binding moiety specifically binding to CD28 is a single chain antibody. In certain embodiments, said antigen-binding moiety specifically binding to CD28 competes for binding to CD28 with an antigen-binding moiety comprising an amino acid sequence of SEQ ID No. 28.

In certain embodiments of the present disclosure, the recombinant adenovirus or the set of recombinant adenoviruses comprise a functional interleukin 2 polypeptide. Many variants and derivatives of interleukin 2 are known in the art, all of which can be used in the context of the present disclosure, see e.g. U.S. Pat. No. 5,116,943, WO2005086751 WO2012062228, WO2012088446, WO2014153111, WO2020020783, WO2020187848, WO2020260270, WO2021146436 and WO2021185362. In certain embodiments, the functional interleukin 2 polypeptide comprises the amino acid sequence of SEQ ID No. 3. In certain embodiments, the functional interleukin 2 polypeptide consists of the amino acid sequence of SEQ ID No. 3. In certain embodiments, the functional interleukin 2 polypeptide comprises the amino acid sequence of SEQ ID No. 3 with one, two, three, four or five amino acid substitutions, insertions or deletions. In certain embodiments, the functional interleukin 2 polypeptide is a variant of the polypeptide consisting of the amino acid sequence of SEQ ID No. 3, wherein said variant is functionally equivalent to the polypeptide consisting of the amino acid sequence of SEQ ID No. 3. In certain embodiments, the functional interleukin 2 polypeptide comprises the amino acid sequence of SEQ ID No. 4. In certain embodiments, the functional interleukin 2 polypeptide consists of the amino acid sequence of SEQ ID No. 4. In certain embodiments, the functional interleukin 2 polypeptide comprises the amino acid sequence of SEQ ID No. 4 with one, two, three, four or five amino acid substitution, insertions or deletions. In certain embodiments, the functional interleukin 2 polypeptide is a variant of the polypeptide consisting of the amino acid sequence of SEQ ID No. 4, wherein said variant is functionally equivalent to the polypeptide consisting of the amino acid sequence of SEQ ID No. 4. In certain embodiments, the functional interleukin 2 polypeptide comprises the amino acid sequence of SEQ ID No. 25. In certain embodiments, the functional interleukin 2 polypeptide consists of the amino acid sequence of SEQ ID No. 25. In certain embodiments, the functional interleukin 2 polypeptide comprises the amino acid sequence of SEQ ID No. 25 with one, two, three, four or five amino acid substitution, insertions or deletions. In certain embodiments, the functional interleukin 2 polypeptide is a variant of the polypeptide consisting of the amino acid sequence of SEQ ID No. 25, wherein said variant is functionally equivalent to the polypeptide consisting of the amino acid sequence of SEQ ID No. 25.

The recombinant adenovirus or the set of recombinant adenoviruses comprise recombinant polypeptides or proteins, that are displayed on said adenoviruses. Said recombinant proteins comprise an antigen-binding moiety specifically binding to CD3, an antigen-binding moiety specifically binding to CD28, or a functional interleukin 2 polypeptide. Said antigen-binding moiety specifically binding to CD3, antigen-binding moiety specifically binding to CD28, or functional interleukin 2 polypeptide may be fused to the other parts of the recombinant protein in any order.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide.

In certain embodiments, the present disclosure relates to a recombinant protein comprising from the N- to the C-terminus
a) an antigen-binding moiety specifically binding to CD3, an antigen-binding moiety specifically binding to CD28, or a functional interleukin 2 polypeptide,
b) a designed ankyrin repeat domain which binds to the knob of an adenovirus, and
c) a trimerization domain.

In other embodiments, the present disclosure relates to a recombinant protein comprising from the N- to the C-terminus
a) a designed ankyrin repeat domain which binds to the knob of the adenovirus,
b) a trimerization domain, and
c) an antigen-binding moiety specifically binding to CD3, an antigen-binding moiety specifically binding to CD28, or a functional interleukin 2 polypeptide.

In specific embodiments, the present disclosure relates to a recombinant protein comprising from the N- to the C-terminus
a) an antigen-binding moiety specifically binding to CD3,
b) a designed ankyrin repeat domain which binds to the knob of the adenovirus, and
c) a trimerization domain. In certain embodiments, said antigen-binding moiety specifically binding to CD3 is a single-chain antibody. In certain embodiments, said antigen-binding moiety specifically binding to CD3 comprises an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17). In certain embodiments, said antigen-binding moiety specifically binding to CD3 competes for binding to CD3 with an antigen-binding moiety comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17).

In specific embodiments, the present disclosure relates to a recombinant protein comprising from the N- to the C-terminus
a) an antigen-binding moiety specifically binding to CD28,
b) a designed ankyrin repeat domain which binds to the knob of the adenovirus, and
c) a trimerization domain. In certain embodiments, said antigen-binding moiety specifically binding to CD28 is a single-chain antibody. In certain embodiments, said antigen-binding moiety specifically binding to CD28 an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24). In certain embodiments, said antigen-binding moiety specifically binding to CD28 competes for binding to CD28 with an antigen-binding moiety comprising an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24).

In specific embodiments, the present disclosure relates to a recombinant protein comprising from the N- to the C-terminus
a) a functional interleukin 2 polypeptide,
b) a designed ankyrin repeat domain which binds to the knob of the adenovirus, and
c) a trimerization domain. In certain embodiments, the functional interleukin 2 polypeptide comprises the amino acid sequence of SEQ ID No. 3. In certain embodiments, the functional interleukin 2 polypeptide consists of the amino acid sequence of SEQ ID No. 3. In certain embodiments, the functional interleukin 2 polypeptide comprises the amino acid sequence of SEQ ID No. 3 with one, two, three, four or five amino acid substitutions, insertions or deletions. In certain embodiments, the functional interleukin 2 polypeptide is a variant of the polypeptide consisting of the amino acid sequence of SEQ ID No. 3, wherein said variant is functionally equivalent to the polypeptide consisting of the amino acid sequence of SEQ ID No. 3. In certain embodiments, the functional interleukin 2 polypeptide comprises the amino acid sequence of SEQ ID No. 4. In certain embodiments, the functional interleukin 2 polypeptide consists of the amino acid sequence of SEQ ID No. 4. In certain embodiments, the functional interleukin 2 polypeptide comprises the amino acid sequence of SEQ ID No. 4 with one, two, three, four or five amino acid substitutions, insertions or deletions. In certain embodiments, the functional interleukin 2 polypeptide is a variant of the polypeptide consisting of the amino acid sequence of SEQ ID No. 4, wherein said variant is functionally equivalent to the polypeptide consisting of the amino acid sequence of SEQ ID No. 4. In certain embodiments, the functional interleukin 2 polypeptide comprises the amino acid sequence of SEQ ID No. 25. In certain embodiments, the functional interleukin 2 polypeptide consists of the amino acid sequence of SEQ ID No. 25. In certain embodiments, the functional interleukin 2 polypeptide comprises the amino acid sequence of SEQ ID No. 25 with one, two, three, four or five amino acid substitutions, insertions or deletions. In certain embodiments, the functional interleukin 2 polypeptide is a variant of the polypeptide consisting of the amino acid sequence of SEQ ID No. 25, wherein said variant is functionally equivalent to the polypeptide consisting of the amino acid sequence of SEQ ID No. 25.

Said recombinant proteins may also comprise more than one of the entities selected from an antigen-binding moiety specifically binding to CD3, an antigen-binding moiety specifically binding to CD28, and a functional interleukin 2 polypeptide.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3 and an antigen-binding moiety specifically binding to CD28.

In other embodiments, the present disclosure relates to a recombinant protein comprising
a) a designed ankyrin repeat domain which binds to the knob of an adenovirus,
b) a trimerization domain, and
c) an antigen-binding moiety specifically binding to CD3 and a functional interleukin 2 polypeptide.

In other embodiments, the present disclosure relates to a recombinant protein comprising
a) a designed ankyrin repeat domain which binds to the knob of an adenovirus,
b) a trimerization domain, and
c) an antigen-binding moiety specifically binding to CD28 and a functional interleukin 2 polypeptide.

In other embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28 and a functional interleukin 2 polypeptide.

The recombinant proteins of the present disclosure comprise a designed ankyrin repeat domain which binds to the knob of a virus or adenovirus. It will be appreciated that any designed ankyrin repeat domain with specificity for the knob of a virus or adenovirus may be used within the spirit of the present disclosure. Exemplified herein is a designed ankyrin repeat domain derived from DARPin 1D3 (Proc. Natl. Acad. Sci. 110, E869-E877 (2013)). DARPin 1D3 binds to the knob of an adenovirus and comprises the amino acid sequence of SEQ ID No. 2. Used herein is 1D3nc, a derivative of 1D3 containing a stabilized C-cap.

It will also be understood that also variants of DARPin 1D3 may used within the spirit of the present disclosure. In other words, the amino acid sequence of such modified DARPin 1D3 does not need to be identical to that of amino acid sequence of SEQ ID No. 2, but may contain amino acids mutations, provided that the function of DARPin 1D3, i.e. binding to the knob of an adenovirus is preserved. Also DARPins different than 1D3, but having the same target specificity, may be used within the scope of the present disclosure. Such new DARPin may for example be selected in a new screening campaign. Also binding entities different than DARPins, i.e. binders based on a different scaffold, but having the same target specificity as 1D3 might be used.

Therefore, in certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said designed ankyrin repeat domain which binds to the knob of an adenovirus is DARPin 1D3.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said designed ankyrin repeat domain which binds to the knob of an adenovirus is or is derived from DARPin 1D3.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said designed ankyrin repeat domain which binds to the knob of an adenovirus is a variant of DARPin 1D3.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said designed ankyrin repeat domain which binds to the knob of an adenovirus comprises the amino acid sequence of SEQ ID No. 2.

In certain embodiments, the present disclosure relates to a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said designed ankyrin repeat domain which binds to the knob of an adenovirus comprising a variant of the amino acid sequence of SEQ ID No. 2.

In certain embodiments, the present disclosure relates to recombinant proteins comprising a designed ankyrin repeat domain that binds to the knob of an adenovirus. The present disclosure can however also be practiced with other viruses. If another virus is used a designed ankyrin repeat domain needs to be selected that binds to the knob of such virus. Therefore, in certain embodiments the present disclosure relates to recombinant proteins comprising
a) a designed ankyrin repeat domain which binds to the knob of the adenovirus,
b) trimerization domain, and
c) said an antigen-binding moiety specifically binding to CD3, said antigen-binding moiety specifically binding to CD28, or said functional interleukin 2 polypeptide.

Viruses, other than adenoviruses of serotype 5, can also be used within the spirit of the present disclosure. For example, such viruses can be engineered to carry the knob of an adenovirus of serotype 5. The recombinant protein disclosed herein, in particular recombinant protein comprising DARPin 1D3, may then be used with such viruses. Therefore, in certain embodiments the present disclosure provides a recombinant virus or a set of recombinant viruses comprising a recombinant protein comprising:
a) an antigen-binding moiety specifically binding to CD3, an antigen-binding moiety specifically binding to CD28, or a functional interleukin 2 polypeptide,
b) a designed ankyrin repeat domain which binds to the knob of an adenovirus, and
c) a trimerization domain,
wherein said designed ankyrin repeat domain binds to the knob of an adenovirus of subtype 5.

It will be understood that also other adenoviral serotypes may be used in the spirit of the present disclosure, including human adenovirus serotype c5 (HAdV-C5), HAd2, HAd3, HAdV-B35, HAdV-D26, as well as hybrids thereof. A list of adenoviruses can be found on the website of the Human Adenovirus Working group (http://hadvwg.gmu.edu). Also, non-human adenoviruses may be used within the scope of the present disclosure, such as the AstraZeneca vaccine chimpanzee adenovirus Y25 (CHAdY25), or non-human adenoviral vectors were developed from bovine (BAd), canine (CAd), chimpanzee (ChAd), ovine (OAd), porcine (PAd), or fowl (FAd).

A preferred virus to be used in the context of the present disclosure is adenovirus of serotype 5. Therefore, in certain embodiments the present disclosure relates to recombinant proteins comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said designed ankyrin repeat domain binds to the knob of an adenovirus of serotype 5.

In certain embodiments, the present disclosure relates to recombinant proteins or uses of such recombinant proteins that comprise a trimerization domain. The trimerization domain is responsible for the formation of trimers. Each monomer of the molecules of the present disclosure comprises a trimerization domain. Principally any trimerization domain may be used, provided it is stable and geometrically fits the knob of the adenovirus used. A preferred trimerization domain is the capsid protein SHP of lambdoid phage 21 (J Mol Biol; 344 (1): 179-93; PNAS 110 (10): E869-77 (2013)).

Therefore, in certain embodiments the present disclosure relates to recombinant proteins comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide, wherein said trimerization domain is the capsid protein SHP of lambdoid phage 21.

In other embodiments the present disclosure relates to recombinant proteins comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide, wherein said trimerization domain is derived from the capsid protein SHP of lambdoid phage 21.

In other embodiments the present disclosure relates to recombinant proteins comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said trimerization domain comprises the amino acid sequence of SEQ ID No. 1.

Also, other trimerization domains known to the skilled person may be used for the formation or trimers. Without being limited other potential trimerization domains include the trimerization domain involved in collagen folding (Int J Biochem Cell Biol 44:21-32 (2012)), the trimerization domain of T4 phage fibritin (PLOS One 7: e43603 (2012)) or the GCN4-based isoleucine zipper (J Biol Chem 290:7436-42 (2015)).

The trimerization domain is responsible for the formation of the trimeric adapter molecules. The trimers disclosed herein are extraordinary stable (J Mol Biol (2004) 344:179-93; PNAS (2013) 110 E869-77). In certain embodiments the trimeric adapter molecules of the present disclosure remain intact in SDS gel electrophoresis. In other embodiments the trimeric adapter molecules are not denatured in SDS gel electrophoresis. In other embodiments the trimeric adapter molecules have a half-life in solution of at least one week, preferably at least two week and even more preferably at least one month.

Therefore, in certain embodiments the present disclosure relates to recombinant proteins comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide,
wherein said trimerization domain has a half-life in solution of at least one week, preferably at least two week and even more preferably at least one month.

In certain embodiments, the present disclosure relates to a trimeric protein consisting of three recombinant proteins as described herein above. In certain embodiments, the present disclosure relates to a trimeric protein, wherein said three recombinant proteins are identical. In certain embodiments, the present disclosure relates to a trimeric protein, wherein said three recombinant proteins comprise an antigen-binding moiety specifically binding to CD3. In certain embodiments, the present disclosure relates to a trimeric protein, wherein said three recombinant proteins comprise an antigen-binding moiety specifically binding to CD28. In certain embodiments, the present disclosure relates to a trimeric protein, wherein said three recombinant proteins comprise a functional interleukin 2 polypeptide.

In certain embodiments, the present disclosure relates to a trimeric protein, wherein said three recombinant proteins comprise
a) an antigen-binding moiety specifically binding to CD3 and an antigen-binding moiety specifically binding to CD28,
b) an antigen-binding moiety specifically binding to CD3 and a functional interleukin 2 polypeptide,
c) an antigen-binding moiety specifically binding to CD28 and a functional interleukin 2 polypeptide, or
d) an antigen-binding moiety specifically binding to CD3, an antigen-binding moiety specifically binding to CD28 and a functional interleukin 2 polypeptide.

In certain embodiments, the present disclosure relates to a trimeric protein, wherein said three recombinant proteins comprise an antigen-binding moiety specifically binding to CD3 and an antigen-binding moiety specifically binding to CD28.

In certain embodiments, the present disclosure relates to a trimeric protein, wherein said three recombinant proteins comprise an antigen-binding moiety specifically binding to CD3 and a functional interleukin 2 polypeptide.

In certain embodiments, the present disclosure relates to a trimeric protein, wherein said three recombinant proteins comprise an antigen-binding moiety specifically binding to CD28 and a functional interleukin 2 polypeptide.

In certain embodiments, the present disclosure relates to a trimeric protein, wherein said three recombinant proteins comprise an antigen-binding moiety specifically binding to CD3, an antigen-binding moiety specifically binding to CD28 and a functional interleukin 2 polypeptide.

The recombinant proteins of the present disclosure are encoded by nucleic acids. Vectors comprising these nucleic acids are used to transfect cells which express the recombinant proteins.

Therefore, in certain embodiments, the present disclosure relates to a nucleic acid encoding a recombinant protein of the present disclosure. The present disclosure also relates to a nucleic acid encoding a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide.

In other embodiments, the present disclosure relates to a vector comprising a nucleic acid encoding a recombinant protein of the present disclosure. The present disclosure also relates to a vector comprising a nucleic acid encoding a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide.

In another embodiments, the present disclosure relates to an adenovirus comprising a nucleic acid encoding a recombinant protein of the present disclosure. In yet another embodiment, the present disclosure relates to an adenovirus comprising a vector comprising a nucleic acid encoding a recombinant protein of the present disclosure. In certain embodiments said adenovirus carries a TAYT mutation. In certain embodiments said adenovirus carries a HVR7 mutation.

In certain embodiments, the present disclosure also relates to an adenoviral vector comprising a nucleic acid encoding a recombinant protein comprising
a) a designed ankyrin repeat domain which binds to the knob of an adenovirus,
b) a trimerization domain, and
c) an antigen-binding moiety specifically binding to CD3, an antigen-binding moiety specifically binding to CD28, or a functional interleukin 2 polypeptide.

The recombinant protein of the present disclosure may also comprise a flexible linker. If the recombinant protein comprising from the N- to the C-terminus
a) an antigen-binding moiety specifically binding to CD3, an antigen-binding moiety specifically binding to CD28, or a functional interleukin 2 polypeptide,
b) a designed ankyrin repeat domain which binds to the knob of an adenovirus, and
c) a trimerization domain,
   then said flexible linker is between said antigen-binding moiety specifically binding to CD3, said antigen-binding moiety specifically binding to CD28, or said functional interleukin 2 polypeptide and said designed ankyrin repeat domain which binds to the knob of an adenovirus.

Therefore, in certain embodiments the present disclosure relates to a recombinant protein comprising from the N- to the C-terminus
a) an antigen-binding moiety specifically binding to CD3, an antigen-binding moiety specifically binding to CD28, or a functional interleukin 2 polypeptide,
b) a flexible linker,
c) a designed ankyrin repeat domain which binds to the knob of an adenovirus, and
d) a trimerization domain.

If the recombinant protein comprising from the N- to the C-terminus
a) a designed ankyrin repeat domain which binds to the knob of the adenovirus,
b) a trimerization domain, and
c) an antigen-binding moiety specifically binding to CD3, an antigen-binding moiety specifically binding to CD28, or a functional interleukin 2 polypeptide,
   then said flexible linker is between said trimerization domain and said antigen-binding moiety specifically binding to CD3, said antigen-binding moiety specifically binding to CD28, or said functional interleukin 2 polypeptide.

Therefore, in certain embodiments the present disclosure relates to a recombinant protein comprising from the N- to the C-terminus
a) a designed ankyrin repeat domain which binds to the knob of the adenovirus,
b) a trimerization domain,
c) a flexible linker, and
d) an antigen-binding moiety specifically binding to CD3, an antigen-binding moiety specifically binding to CD28, or a functional interleukin 2 polypeptide.

Principally any flexible linker can be used within the spirit of the present disclosure. Certain preferred flexible linkers are glycine-serine linkers. A particularly preferred flexible linker is a (Gly₄Ser)₄-linker.

Therefore, in certain embodiments the present disclosure relates to a recombinant protein comprising from the N- to the C-terminus
a) an antigen-binding moiety specifically binding to CD3, an antigen-binding moiety specifically binding to CD28, or a functional interleukin 2 polypeptide,
b) a flexible linker,
c) a designed ankyrin repeat domain which binds to the knob of an adenovirus, and
d) a trimerization domain,
   wherein said flexible linker is a glycine-serine linker.

In other embodiments the present disclosure relates to a recombinant protein comprising from the N- to the C-terminus
a) an antigen-binding moiety specifically binding to CD3, an antigen-binding moiety specifically binding to CD28, or a functional interleukin 2 polypeptide,
b) a flexible linker,
c) a designed ankyrin repeat domain which binds to the knob of an adenovirus, and
d) a trimerization domain,
   wherein said flexible linker is a (Gly₄Ser)₄-linker.

The recombinant protein of the present disclosure may also comprise a short linker. The short linker is located between the designed ankyrin repeat domain which binds to the knob of an adenovirus and the trimerization domain.

Therefore, in certain embodiments the present disclosure relates to a recombinant protein comprising from the N- to the C-terminus
a) an antigen-binding moiety specifically binding to CD3, an antigen-binding moiety specifically binding to CD28, or a functional interleukin 2 polypeptide,
b) a designed ankyrin repeat domain which binds to the knob of an adenovirus,
c) a short linker, and
d) a trimerization domain.

In other embodiments the present disclosure relates to a recombinant protein comprising from the N- to the C-terminus
a) a designed ankyrin repeat domain which binds to the knob of the adenovirus,
b) a short linker,
c) a trimerization domain, and
d) an antigen-binding moiety specifically binding to CD3, an antigen-binding moiety specifically binding to CD28, or a functional interleukin 2 polypeptide.

The short linker does not necessarily be present. Possible short linkers of the present disclosure are linkers which are no longer than four, no longer than three, no longer than two or only one amino acid long. A preferred short linker is glycine. Another preferred short linker is glycine-alanine. Most preferably the short linker is absent.

The recombinant proteins of the present disclosure can be expressed in prokaryotic cells, such as Escherichia coli, and in eukaryotic cells. Preferred eukaryotic cells are CHO cells. Other preferred eukaryotic cells are HEK293 cells, HEK293-T cells, HEK293-F cells, CHO-S cells and Sf9 cells. Therefore, in certain embodiments the present disclosure provides a eukaryotic cell expressing the recombinant protein of the present disclosure. In certain other the present disclosure provides a CHO cell expressing the recombinant protein of the present disclosure. In certain embodiments, the present disclosure relates to a eukaryotic cell expressing a recombinant protein comprising
a) an antigen-binding moiety specifically binding to CD3, said antigen-binding moiety specifically binding to CD28, or said functional interleukin 2 polypeptide,
b) a designed ankyrin repeat domain which binds to the knob of the adenovirus, and
c) a trimerization domain.

In certain embodiments, the present disclosure relates to a CHO cell expressing a recombinant protein comprising
a) an antigen-binding moiety specifically binding to CD3, said antigen-binding moiety specifically binding to CD28, or said functional interleukin 2 polypeptide,
b) a designed ankyrin repeat domain which binds to the knob of the adenovirus, and
c) a trimerization domain.

The recombinant proteins of the present disclosure, the nucleic acids encoding the recombinant proteins and trimeric proteins of the present disclosure, the vectors containing the nucleic acids of the present disclosure and the recombinant adenoviruses or set of recombinant adenoviruses containing the recombinant proteins, the nucleic acids or the vectors of the present disclosure have numerous uses, such as the use in an adenoviral delivery system. Therefore, in certain embodiments the present disclosure provides the use of the recombinant proteins of the present disclosure in an adenoviral delivery system. In other embodiments the present disclosure provides the use of the nucleic acids encoding the recombinant proteins of present disclosure in an adenoviral delivery system. In other embodiments the present disclosure provides the use of the vectors containing the nucleic acids of the present disclosure in an adenoviral delivery system. In other embodiments the present disclosure provides the use of the adenoviruses containing the recombinant proteins, the nucleic acids or the vectors of the present disclosure in an adenoviral delivery system.

The recombinant proteins of the present disclosure, the nucleic acids encoding the recombinant proteins and trimeric proteins of the present disclosure, the vectors containing the nucleic acids of the present disclosure and the recombinant adenoviruses or set of recombinant adenoviruses containing the recombinant proteins, the nucleic acids or the vectors of the present disclosure can also be used for the transduction of immune cells. Therefore, in certain embodiments the present disclosure provides the use of the recombinant proteins of the present disclosure for the transduction of immune cells. Preferably said immune cells are T cells. Also, preferably, said immune cells are NK cells. Therefore, in certain embodiments the present disclosure provides the use of the recombinant proteins of the present disclosure for the transduction of T cells. In other embodiments the present disclosure provides the use of the recombinant proteins of the present disclosure for the transduction of NK cells. In other embodiments the present disclosure provides the use of the nucleic acids encoding the recombinant proteins of present disclosure for the transduction of T cells. In other embodiments the present disclosure provides the use of the nucleic acids encoding the recombinant proteins of present disclosure for the transduction of NK cells. In other embodiments the present disclosure provides the use of the vectors containing the nucleic acids of the present disclosure for the transduction of T cells. In other embodiments the present disclosure provides the use of the vectors containing the nucleic acids of the present disclosure for the transduction of NK cells. In other embodiments the present disclosure provides the use of the adenoviruses containing the recombinant proteins, the nucleic acids or the vectors of the present disclosure for the transduction of T cells. In other embodiments the present disclosure provides the use of the adenoviruses containing the recombinant proteins, the nucleic acids or the vectors of the present disclosure for the transduction of NK cells.

In certain embodiments the present disclosure relates to the use of a recombinant adenovirus or a set of recombinant adenoviruses for the transduction of activated immune cells, wherein said recombinant adenovirus or said set of recombinant adenoviruses display
a) an antigen-binding moiety specifically binding to CD3, and
b) a functional interleukin 2 polypeptide. In certain embodiments said immune cells are T cells. In certain embodiments said immune cells are NK cells.

In certain embodiments the present disclosure relates to the use of a recombinant adenovirus or a set of recombinant adenoviruses for the transduction of pre-activated immune cells, wherein said recombinant adenovirus or said set of recombinant adenoviruses display
a) an antigen-binding moiety specifically binding to CD3, and
b) a functional interleukin 2 polypeptide. In certain embodiments said immune cells are T cells. In certain embodiments said immune cells are NK cells.

The recombinant proteins of the present disclosure, the nucleic acids encoding the recombinant proteins and trimeric proteins of the present disclosure, the vectors containing the nucleic acids of the present disclosure and the recombinant adenoviruses or set of recombinant adenoviruses containing the recombinant proteins, the nucleic acids or the vectors of the present disclosure can also be used in medicine. Therefore, in certain embodiments the present disclosure provides the use of the recombinant proteins of the present disclosure in medicine. In other embodiments the present disclosure provides the use of the nucleic acids encoding the recombinant proteins of the present disclosure in medicine. In other embodiments the present disclosure provides the use of the vectors containing the nucleic acids of the present disclosure in medicine. In other embodiments the present disclosure provides the use of the adenoviruses containing the recombinant proteins, the nucleic acids or the vectors of the present disclosure in medicine.

In certain embodiments the present disclosure provides a method to treat a patient, said method comprising administering to a patient in need of a recombinant protein of the present disclosure. In certain embodiments the present disclosure provides a method to treat a patient, said method comprising administering to a patient in need of a nucleic acid encoding a recombinant protein of the present disclosure. In certain embodiments the present disclosure provides a method to treat a patient, said method comprising administering to a patient in need thereof a vector containing a nucleic acid of the present disclosure. In certain embodiments the present disclosure provides a method to treat a patient, said method comprising administering to a patient in need thereof a recombinant adenovirus or a set of recombinant adenoviruses containing a recombinant protein, a nucleic acid or a vector of the present disclosure.

Principally, the recombinant proteins of the present disclosure, the nucleic acids encoding the recombinant proteins of the present disclosure, the vectors containing the nucleic acids of the present disclosure, the recombinant adenovirus or set of recombinant adenoviruses displaying or containing the recombinant proteins, the nucleic acids or the vectors of the present disclosure, and the eukaryotic cells containing the recombinant adenovirus or set of recombinant adenoviruses of the present disclosure can be used in the treatment or prevention of any disease or disorder.

In certain embodiments the present disclosure encompasses:
1. A recombinant adenovirus or a set of recombinant adenoviruses displaying
   a) an antigen-binding moiety specifically binding to CD3, and/or
   b) an antigen-binding moiety specifically binding to CD28, and/or
   c) a functional interleukin 2 polypeptide.
2. The recombinant adenovirus or the set of recombinant adenoviruses according to claim 1, wherein said adenoviruses are capable of transducing T cells.
3. The recombinant adenovirus or the set of recombinant adenoviruses according to any one of the preceding claims, wherein said antigen-binding moiety specifically binding to CD3 is a single-chain antibody specifically binding to CD3.
4. The recombinant adenovirus or the set of recombinant adenoviruses according to claim 3, wherein said single chain antibody specifically binding to CD3 comprises an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17).
5. The recombinant adenovirus or the set of recombinant adenoviruses according to any one of the preceding claims, wherein said antigen-binding moiety specifically binding to CD28 is a single-chain antibody specifically binding to CD28.
6. The recombinant adenovirus or the set of recombinant adenoviruses according to claim 5, wherein said single chain antibody specifically binding to CD28 comprises an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNFDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24).
7. The recombinant adenovirus or the set of recombinant adenoviruses according to any one of the preceding claims, wherein said functional interleukin 2 polypeptide comprises the amino acid sequence of SEQ ID No. 3, 4 or 25.
8. The recombinant adenovirus or the set of recombinant adenoviruses according to any one of the preceding claims, wherein said an antigen-binding moiety specifically binding to CD3, said antigen-binding moiety specifically binding to CD28, and said functional interleukin 2 polypeptide are displayed on the knob of said adenoviruses.
9. The recombinant adenovirus or the set of recombinant adenoviruses according to any one of the preceding claims, wherein said an antigen-binding moiety specifically binding to CD3, said antigen-binding moiety specifically binding to CD28, and said functional interleukin 2 polypeptide are comprised in a recombinant protein comprising from the N- to the C-terminus
   a) said antigen-binding moiety specifically binding to CD3, said antigen-binding moiety specifically binding to CD28, or said functional interleukin 2 polypeptide,
   b) a designed ankyrin repeat domain which binds to the knob of the adenovirus, and
   c) a trimerization domain.
10. The recombinant adenovirus or the set of recombinant adenoviruses according to claim 9, wherein said designed ankyrin repeat domain that binds to a knob of an adenovirus comprises the amino acid sequence of SEQ ID No. 2.
11. The recombinant adenovirus or the set of recombinant adenoviruses according to any one of the preceding claims, wherein said adenovirus is of adenovirus serotype 5 or wherein said adenovirus comprises a knob of an adenovirus of serotype 5.
12. The recombinant adenovirus or the set of recombinant adenoviruses according to any one of claims 9-11, wherein said trimerization domain is or is derived from the capsid protein SHP of lambdoid phage 21.
13. The recombinant adenovirus or the set of recombinant adenoviruses according to claim 12, wherein said trimerization domain comprises the amino acid sequence of SEQ ID No. 1.
14. A recombinant protein comprising from the N- to the C-terminus
   d) an antigen-binding moiety specifically binding to CD3, an antigen-binding moiety specifically binding to CD28, or a functional interleukin 2 polypeptide,
   e) a designed ankyrin repeat domain which binds to the knob of an adenovirus, and
   f) a trimerization domain.
15. A trimeric protein consisting of three recombinant proteins according to claim 14.
16. The trimeric protein according to claim 15, wherein said three recombinant proteins are identical.
17. The trimeric protein according to claim 15, where said recombinant proteins comprise an antigen-binding moiety specifically binding to CD3, an antigen-binding moiety specifically binding to CD28, or a functional interleukin 2 polypeptide.
18. The trimeric protein according to claim 15, where said three recombinant proteins are different.
19. The trimeric protein according to claim 18, where said three recombinant proteins comprise
   a) an antigen-binding moiety specifically binding to CD3 and an antigen-binding moiety specifically binding to CD28,
   b) an antigen-binding moiety specifically binding to CD3 and a functional interleukin 2 polypeptide,
   c) an antigen-binding moiety specifically binding to CD28 and a functional interleukin 2 polypeptide, or
   d) an antigen-binding moiety specifically binding to CD3, an antigen-binding moiety specifically binding to CD28 and a functional interleukin 2 polypeptide.
20. A nucleic acid encoding a recombinant protein according to claim 14.
21. Use of the recombinant adenovirus or the set of recombinant adenoviruses according to any one of claims 1-13, the recombinant protein according to claim 14 or the trimeric protein according to claim 15-19 for the transduction of immune cells, preferably T cells.
22. Use of the recombinant adenovirus or the set of recombinant adenoviruses according to any one of claims 1-13, the recombinant protein according to claim 14 or the trimeric protein according to claim 15-19 for use in medicine.
23. An eukaryotic cell expressing or producing a recombinant adenovirus or the set of recombinant adenoviruses according to any one of claims 1-13

In certain embodiments, the present disclosure relates to a recombinant protein comprising
a) an antigen-binding moiety specifically binding to CD3,
b) a designed ankyrin repeat domain which binds to the knob of an adenovirus, and
c) a trimerization domain.

In certain embodiments, the present disclosure relates to a recombinant protein comprising
a) an antigen-binding moiety specifically binding to CD3,
b) a designed ankyrin repeat domain which binds to the knob of an adenovirus, and
c) a trimerization domain,
   wherein said antigen-binding moiety specifically binding to CD3 is a single-chain antibody specifically binding to CD3.

In certain embodiments, the present disclosure relates to a recombinant protein comprising
a) an antigen-binding moiety specifically binding to CD3,
b) a designed ankyrin repeat domain which binds to the knob of an adenovirus, and
c) a trimerization domain,
   wherein said antigen-binding moiety specifically binding to CD3 is a single-chain antibody specifically comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17).

In certain embodiments, the present disclosure relates to a recombinant protein comprising
a) an antigen-binding moiety specifically binding to CD3,
b) a designed ankyrin repeat domain which binds to the knob of an adenovirus, and
c) a trimerization domain,
   wherein said antigen-binding moiety specifically binding to CD3 is a single-chain antibody specifically comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17), and
   wherein said designed ankyrin repeat domain that binds to a knob of an adenovirus comprises the amino acid sequence of SEQ ID No. 2.

In certain embodiments, the present disclosure relates to a recombinant protein comprising
a) an antigen-binding moiety specifically binding to CD3,
b) a designed ankyrin repeat domain which binds to the knob of an adenovirus, and
c) a trimerization domain,
   wherein said antigen-binding moiety specifically binding to CD3 is a single-chain antibody specifically comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17),
   wherein said designed ankyrin repeat domain that binds to a knob of an adenovirus comprises the amino acid sequence of SEQ ID No. 2, and
   wherein said trimerization domain is or is derived from the capsid protein SHP of lambdoid phage 21.

In certain embodiments, the present disclosure relates to a recombinant protein comprising
a) an antigen-binding moiety specifically binding to CD3,
b) a designed ankyrin repeat domain which binds to the knob of an adenovirus, and
c) a trimerization domain,
   wherein said antigen-binding moiety specifically binding to CD3 is a single-chain antibody specifically comprising an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID No. 16), and an LCDR3 of amino acid sequence QQGNTLPWT (SEQ ID No. 17),
   wherein said designed ankyrin repeat domain that binds to a knob of an adenovirus comprises the amino acid sequence of SEQ ID No. 2, and
   wherein said trimerization domain comprises the amino acid sequence of SEQ ID No. 1.

### EXAMPLES

### Example 1: General Experimental Procedures

### Human Blood Samples

Buffy coats from human donors were acquired from the Blutspende Bern, Bern, Switzerland. After Ficoll-Paque (GE Healthcare) gradient separation PBMC cells were aliquoted and frozen to be thawed before each assay.

After thawing either PBMCs were directly used in assays or T cells were first isolated using a Pan-T cell isolation kit from Miltenyi Biotech (Cat number 130-096-535) as described by the manufacturer, or Treg cells were first isolated using a T regulatory CD4+ CD25+ cell isolation kit from Miltenyi Biotech (Cat number 130-091-301) as described by the manufacturer.

For activation of immune cells, cell were put in RPMI 1640 at 0.5×10⁶ cells/ml and supplemented with a 3:1 ratio of Dynabeads Human T-Expander CD3/CD28 (ThermoFisher, Cat number 11141D) to T cells, 50 IU/ml human IL-2 IS (Miltenyi Biotech, Cat number 130-097-743), recombinant adenovirus displaying a functional interleukin 2 polypeptide and incubated for at least 48 hours thereafter, sometimes as long as 120 hours.

Jurkat E_6 cells were obtained from ATCC (Order no: TIB-152) and maintained in R10 Medium (RPMI 1640, 10% FCS, 1% Penicillin-streptomycin) at a density of 0.5 to 2×10⁶ cells/ml. The ethical approval for the use of human fetal liver tissue as well as peripheral blood samples of healthy adult volunteers were obtained from the cantonal ethical committee of Zurich, Switzerland (protocol no. KEK-StV-Nr.19/08). Buffy coats from HDs were acquired from the Blutspende Zurich, Zurich, Switzerland. After Ficoll-Paque (GE Healthcare) gradient separation donor cells were aliquoted and frozen to be thawed before each assay. To use pre-activated cells, thawed PBMCs were put in R10 culture at 1×10⁶ cells/ml and supplemented with a 1:1 ratio of Dynabeads Human T-Expander CD3/CD28 (ThermoFisher) and 200 IU/ml human IL-2 (PeproTech) and incubated for 24 h. Afterwards, magnetic beads were removed from activated cells using a magnetic strip and washing twice with R10. Non-pre-activated cells were cultured in R10 supplemented with 50 IU/ml IL-2.

### Viral Vector Generation

The replication-deficient HAdV-C5 helper virus (HV) contains an E1/E3 deletion, a loxP-flanked packaging signal and 4 mutations in the hypervariable region 7 (HVR7) of the hexon protein (1421G, T423N, E424S and L426Y). The HV was generated as previously described (Nat. Commun. 9, 450 (2018)) or ordered from Vector Biolabs (Malvern, PA/USA). The helper-dependent adenovirus genome containing no adenoviral genes, but the adenoviral packaging sequences was propagated using the above HV as described by Brücher et al. (Mol Ther Methods Clin Dev (2021) 20:572-86). In short, a Cre-expressing HEK293 cell line was transfected with the helper-dependent genomes and co-transduced with the helper HAdV-C5 vector for replication. Purification was performed via density separation. Similarly, the cell line 116 was transfected with the reporter plasmid containing the HAdV-C5 packaging signal and co-transduced with a helper HAdV-C5 for replication. Purification was performed via two CsCl gradients at 250,000 g.

### Expression and Purification of the Recombinant Proteins and Adapter Molecules

The trimeric adapters were cloned into the mammalian expression plasmid pcDNA3.1. Alternatively other plasmids, such as pTwist CMV WPRE Neo can be used). DNA synthesis and expression plasmid construct assembly was carried out by a commercial vendor (TWIST, San Francisco, USA. Alternatively Gibson assembly strategies can be used (Gibson et al., Nat. Methods 343-5 (2009)). The adapter contained an N-terminal HSA leader peptide, an 3C-cleavable His6- and Flag-tag. Generally any N- or C-terminal peptide affinity purification tag (e.g. polyhistidine-tag, Strep-tag) or an epitope tag (e.g. FLAG-tag, polyhistine-tag, Myc-tag), or a combination thereof, can be used. Likewise, any proteolytic cleavage site (e.g. for furin, thrombin, TEV, or the like) may be introduced for affinity purification or epitope tag removal. The adapters were cloned into pcDNA3.1, as previously described (Adv. Cancer Res. 115, 39-67 (2012)). The retargeting domain is flanked by a BamHI and an HindIII site for ready exchange of the domain. Adapters were expressed in CHO-S cells as described (Protein Expr. Purif. 92, 67-76 (2013)). Generally, any mammalian expression host can be used, e.g. HEK293. Following seven days expression, supernatants were 1:1000 dialyzed in PBS pH 7.4 using dialysis tubes with a MWCO cutoff of 12-14 kDa at 4° C. During 24 h, the buffer was exchanged four times 1:10. Dialyzed supernatants were subjected 2.5 ml equilibrated nickel-nitrilotriacetic acid (Ni-NTA) resin (Thermo Fisher) in a PD-10 column (Merck Millipore). All columns were washed with 5 column volumes 20 mM imidazole, 10% glycerol, PBS pH 8.0 and then additionally with 5 column volumes of 500 mM NaCl, 50 mM Tris HCl pH 8.0. The samples were then eluted using 0.7 M imidazole in PBS pH 8.0, followed by subsequent 3C cleavage (GenScript) of the tags during dialysis against 20 mM Hepes at pH 7.4. An additional purification step included an anion exchange chromatography using a MonoQ column (GE Healthcare). Purified protein was dialyzed four times 1:100 in 24 h in endotoxin-free PBS (Merck Millipore) and then shock frozen in liquid nitrogen and stored at −80° C. until usage.

### In Vitro Assays

PBMC were thawed 24 h prior transduction and taken in culture. Cells were distributed in round bottom 96-well plates with 5×10⁴ cells/well in 100 μl R10. Adenoviral vectors were incubated for 1 h at 4° C. with adapters coupled to a α-CD3 scFv and/or IL-2 and/or α-CD28 scFv (termed “retargeted”), or a blocking adapter containing the consensus DARPin E2_5 (J Mol Biol (2003) 332:489-503) (termed “non-binding adapter”). Optionally, previously the described shield (Schmid et al. 2018 Nat. Commun.) was added (termed “shielded”). As a control, adenoviral vectors without adapters (termed “untargeted vectors”) were incubated with PBS. The ratio total adapter to fiber-knob was 1:32.5 and for the shield 1:5 per hexon. 2×10⁴ optical viral particles were used per cell. Viral particle-containing supernatants were removed 16 h after transduction and replaced by fresh culture medium. Transduced transgene activity was determined by flow cytometry 48 h after transduction.

### Specificity Assays

For competitions assays, the procedure followed the in vitro retargeting assays, however ratios of adapter to vector knob were increased in the indicated steps. For CD3 specificity, 2*10⁵ Jurkat T cells were electroporated in OptiMEM with a total of 90 pmol RNP (30 pmol CD3D RNP, 30 pmol CD3E RNP, 30 pmol CD3G RNP) in one well of a 16-well strip (Lonza, from Cat. V4XP-3032) with the pulse CL-120 using the 4D Nucleofector (Lonza). S.p. Cas9 (IDT, Cat. 1081059), crRNA and trRNA (IDT, Cat. 1072533) were ordered from IDT. Electroporated Jurkat cells were expanded for 10 days. Cells were subsequently purified by negative CD3 MACS selection (Miltenyi, Cat. 130-050-101) using the autoMACS machine (Miltenyi). Following crRNA sequences listed below were used to delete functional CD3 expression:

CD3D:
(SEQ ID No. 26)
AAACGCAUCCUGGACCCACG
CD3E:
(SEQ ID No. 27)
AGAUAAAAGUUCGCAUCUUC
CD3G:
(SEQ ID No. 28)
UACACUGAUACAUCCCUCGA

### Animal Experiments

NOD-scid γ_{c}^{null} (NSG) mice were obtained from Jackson Laboratories and bred and maintained under specific pathogen-free conditions at the Institute of Experimental Immunology, University of Zurich. Both male and female mice were used for experiments at the age of 12-15 weeks. Retargeted vectors were injected intraperitoneally. 48 h after injection, cells were harvested by intraperitoneal lavage with 10 ml of PBS.

The reconstitution of newborn NSG mice with human immune system components was performed as previously described. Before the start of subsequent experiments, the level of human immune cell reconstitution was checked in blood by flow cytometry. Retargeted vector was injected intravenously. Mice were sacrificed 48 hours after injection and blood, spleen, liver and bone marrow were harvested.

### In Vitro Transduction

PBMCs, T cells or Treg cells were thawed and directly taken into experiments. Cells were distributed in flat bottom 96-well plates with 5×10⁴ cells/well in 100 μl RPMI 1640. Adenoviral vectors were incubated for 1 h at 4° C. with adapters coupled to a α-CD3 scFv and/or IL-2 and/or α-CD28 scFv (termed “retargeted”), or a blocking adapter containing the consensus DARPin E2_5 (J Mol Biol (2003) 332:489-503) (termed “blocking adapter”). Optionally, previously the described shield (Schmid et al. 2018 Nat. Commun.) was added (termed “shielded”). As a control, adenoviral vectors without adapters (termed “untargeted vectors”) were incubated with PBS. The ratio total adapter to fiber-knob was variable depending on specific experiments and for the shield 1:5 per hexon. Genomic viral particles were also variable depending on specific experiments. Activation of immune cells was performed on some experiments as mentioned on below on the further Examples given, according to the Human Blood samples experimental protocol. Transduced transgene activity was determined by flow cytometry as early as 48 h after transduction.

### Flow Cytometry

For in vitro assays cells were centrifuged at 500-750 g for 5 min and supernatant was discarded. The pellet was resuspended in PBS containing 1% BSA and 0.05% azide. Cells were then kept at 4° C. in the dark for 30 min and washed twice with PBS. Cells were then resuspended in PBS containing 2% PFA and fixed for 15 min at room temperature. Remaining PFA was then quenched by adding 5× of the fixation volume of PBS containing 1% BSA and 0.05% azide.

Dead cells were stained using the fixable viability dye Zombie (BioLegend) for 15 min at room temperature, followed by blocking of Fc receptors with TruStain FcX (BioLegend) for 20 min at 4° C. Following this, cell surface proteins were stained for 20 min at 4° C. Nuclear proteins were stained for 60 min at room temperature after permeabilization and fixation (Regulatory T cell Staining Kit, eBioscience). Samples were analyzed on a BD LSRFortessa™ flow cytometer (BD Biosciences). Commercially available antibodies were used in FACS experiments. GFP was detected as the transgene transduced.

In case of in vivo analysis, erythrocytes in blood samples were lysed three times with NH₄Cl. Spleens were mechanically mashed through a 70 μm cell strainer before isolation of lymphocytes using density gradient centrifugation. Bone marrow was extracted by centrifugation of bones that were cut open at one end and directly used for antibody labeling. Cells were stained with the appropriate amount of antibodies for 20 min at 4° C. in PBS and fixed with PBS+2% PFA for 30 min at 4° C. Samples were analyzed on a BD LSRFortessa™ flow cytometer (BD Biosciences). Commercially available antibodies were used in FACS experiments.

### FACS Sorting

Lymphocytes isolated from the spleen were stained for anti-human CD19, anti-human CD45, anti-human CD3 and anti-murine CD45 as described. 100.000 CD3⁺ and 100.000 CD19⁺ events were sorted with a FACSAria Ill cell sorter using the 100-micron nozzle and BD FACS Diva Software (BD Biosciences). Purity of the samples was confirmed after the sort by re-analysis.

### Flow Cytometry Analysis

Cells were centrifuged at 750 g for 5 min and resuspended in PBS containing 1% BSA and 0.05% azide containing all used antibodies. Cells were then kept at 4° C. in the dark for 30 min and washed twice with PBS. Cells were then resuspended in PBS containing 2% PFA and fixed for 20 min at room temperature. Remaining PFA was then quenched by adding 5× of the fixation volume of PBS containing 1% BSA and 0.05% azide.

Quantitative Polymerase Chain Reaction Analysis (qPCR)

Viral and cellular genomes were extracted from tissues using the DNeasy Blood & Tissue Kit (Qiagen, #69506) following manufacturer's protocol. DNA amounts were quantified by qPCR with specific primers for hB2M and adenoviral hexon protein respectively. To investigate the amount of viral vector genomes 5′-GAA TAA CAA GTT TAG AAA CCC CAC GGT GG-3′ (SEQ ID No. 29) and 5′-GTT TGA CCT TCG ACA CCT ATT TGA ATA CCC-3 (SEQ ID No. 30) were used resulting in a 149 bp long amplicon. For cellular genomes of human origin, hB2M 5′-GGA ATT GAT TTG GGA GAG CAT C-3′ (SEQ ID No. 31) and 5′-CAG GTC CTG GCT CTA CAA TIT ACT AA-3′ (SEQ ID No. 32) were used to result in a 79 bp long amplicon. Specific probes were as follows: for the hexon (5′-CGG CGT GCT GGA CAG G-3′; SEQ ID No. 33) and hB2M (5′-AGT GTG ACT GGG CAG ATC ATC CAC CTT C-3′; SEQ ID No. 34). Reactions were performed using PrimeTime Gene Expression Master Mix (IDT, 105571) and signals were normalized to the passive dye rhodamine-X (ROX). All qPCR primer and probes were generated with the double quench technology by IDT. The qPCR reaction was performed and analyzed as described previously (Molecular Therapy—Methods and Clinical Development (2021) 20:572-86).

### Recombinant Adapter Molecules

The following recombinant adapter molecules were used for exemplification of the present invention.

The recombinant adapter molecule comprising the functional interleukin 2, polypeptide had the following amino acid sequence:

(SEQ ID No. 6)
MKWVTFISLLFLFSSAYSDYKDDDDKHHHHHHGGGGSLEVLFQGPGSAPT
SSSTKKTQLQLEHLLLDLQMILNGINNYKNPKLTRMLTFKFYMPKKATEL
KHLQCLEEELKPLEEVLNLAQSKNFHLRPRDLISNINVIVLELKGSETTF
MCEYADETATIVEFLNRWITFCQSIISTLTKLGGGGSGGGGSGGGGSGGG
GSRSDLGKKLLEAARAGQDDEVRILMANGADANAYDHYGRIPLHMAAAVG
HLEIVEVLLRNGADVNAVDINGTTPLHLAASLGHLEIVEVLLKYGADVNA
KDATGITPLYLAAYWGHLEIVEVLLKHGADVNAQDKFGKTPFDLAIDNGN
EDIAEVLQGVRIFAGNDPAHTATGSSGISSPTPALTPLMLDEATGKLVVW
DGQKAGSAVGILVLPLEGTETALTYYKSGTFATEAIHWPESVDEHKKANA
FAGSALSHAALP

Alternatively a recombinant adapter molecule comprising a stability enhanced functional interleukin. 2 polypeptide with the following amino acid sequence can be used:

(SEQ ID No. 7)
ATMETDTLLLWVLLLWVPGSTGRWSHPQFEKSHHHHHHHHENLYFQSGSP
TSSSTKKTQLQLEHLLLDLQMILNGINNYKNPKLTRMLTFKFYMPKKATE
LKHLQCLEEELKPLEEVLNLAQSKNFHLRPRDLISNINVIVLELKGSETT
FMCEYADETATIVEFLNRWITFSQSIISTLTKLGGGGSGGGGSGGGGSGG
GGSRSDLGKKLLEAARAGQDDEVRILMANGADANAYDHYGRTPLHMAAAV
GHLEIVEVLLRNGADVNAVDINGTTPLHLAASLGHLEIVEVLLKYGADVN
AKDATGITPLYLAAYWGHLEIVEVLLKHGADVNAQDKEGKTPFDLAIDNG
NEDIAEVLQGVRIFAGNDPAHTATGSSGISSPTPALTPLMLDEATGKLVV
WDGQKAGSAVGILVLPLEGTETALTYYKSGTFATEAIHWPESVDEHKKAN
AFAGSALSHAALP

The full length anti-CD3 adapter had the following amino acid sequence:

(SEQ ID No. 8)
GPGSDIQMTQSPSSLSASVGDRVTITCRASQDIRNYLNWYQQKPGKAPKL
LIYYTSRLESGVPSRFSGSGSGTDYTLTISSLQPEDFATYYCQQGNILPW
TFGQGTKVEIKRGGGGSGGGGSGGGGSEVQLVESGGGLVQPGGSLRLSCA
ASGYSFTGYTMNWVRQAPGKGLEWVALINPYKGVSTYNQKFKDRFTISVD
KSKNTAYLQMNSLRAEDTAVYYCARSGYYGDSDWYFDVWGQGTLVTVSSK
LGGGGSGGGGSGGGGSGGGGSRSDLGKKLLEAARAGQDDEVRILMANGAD
ANAYDHYGRTPLHMAAAVGHLEIVEVLLRNGADVNAVDINGTTPLHLAAS
IGHLEIVEVLLKYGADVNAKDATGITPLYLAAYWGHLEIVEVLLKHGADV
NAQDKFGKTPFDLAIDNGNEDIAEVLQGVRIFAGNDPAHTATGSSGISSP
TPALTPLMLDEATGKLVVWDGQKAGSAVGILVLPLEGTETALTYYKSGTF
ATEAIHWPESVDEHKKANAFAGSALSHAALP

The full length anti-CD28 adapter had the following amino acid sequence:

(SEQ ID No. 9)
GPGSDIQMTQSPSSLSASVGDRVTITCHASQNIYVWLNWYQQKPGKAPKL
LIYKASNLHTGVPSRFSGSGSGTDFTLTISSLQPEDFATYYCQQGQTYPY
TFGGGTKVEIKRGGGGSGGGGSGGGGSQVQLVQSGAEVKKPGASVKVSCK
ASGYTFTSYYIHWVRQAPGQGLEWIGCIYPGNVNTNYNEKFKDRATLTVD
TSISTAYMELSRLRSDDTAVYFCTRSHYGLDWNFDVWGQGTTVTVSSKLG
GGGSGGGGSGGGGSGGGGSRSDLGKKLLEAARAGQDDEVRILMANGADAN
AYDHYGRTPLHMAAAVGHLEIVEVLLRNGADVNAVDTNGTTPLHLAASLG
HLEIVEVLLKYGADVNAKDATGITPLYLAAYWGHLEIVEVLLKHGADVNA
QDKFGKTPFDLAIDNGNEDIAEVLQGVRIFAGNDPAHTATGSSGISSPTP
ALTPLMLDEATGKLVVWDGQKAGSAVGILVLPLEGTETALTYYKSGTFAT
EAIHWPESVDEHKKANAFAGSALSHAALP

The flexible linker of the exemplified adapter molecules had the following amino acid sequence:

(SEQ ID No. 10)
GGGGSGGGGSGGGGSGGGGS

### Example 2: Transduction Efficiency is Very Low without Activation of T Cells

In this experiment, different adapter molecules were tested for the capability to improve the transduction of non-activated T cells. Tested were adenoviruses (100 genomic particles per cell) displaying adapters comprising an anti-CD3 single chain antibody, adenoviruses displaying adapters comprising an anti-CD28 single-chain antibody, adenoviruses displaying adapters comprising a functional IL-2 polypeptide and adenoviruses displaying a combination of the three aforementioned adapter molecules (1:1:1 ratio). As controls a blocked adapter and a non-targeted adapter were used. The blocked adapter is the E2-5 adapter, an adapter which is commonly used as a control adapter which lacks any target specificity (see e.g. J Mol Biol (2011) 405:410-26). The non-target vector refers to a vector which is devoid of a targeting moiety.

In vitro transduction, flow cytometry and recombinant adapter molecules are as stated in Example 1.

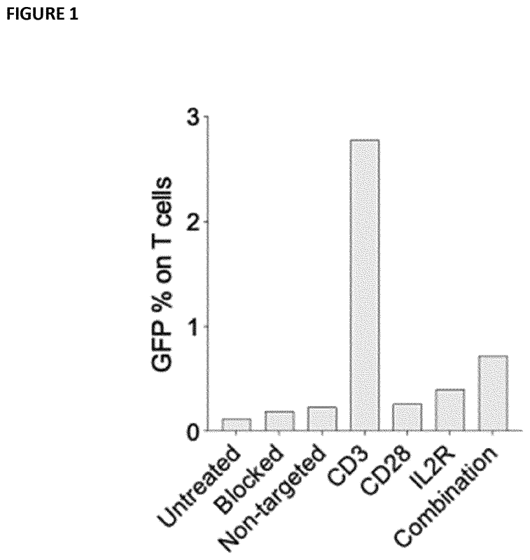

Measured was the percentage of T cells expressing GFP. Results are shown in FIG. 1. All experimental conditions tested, including all adapters tested, led to a transduction efficacy of less than 3%.

### Example 3: High-Efficient Transduction of T Cells Using Virus Like Particles

In this experiment, the efficacy to transduce activated T cells was tested with various construct and conditions. In contrast to Example 2, T cell were activated with Dynabeads (Human T-Expander CD3/CD28; ThermoFisher) and human recombinant human IL-2 (50 IU per ml; Miltenyi Biotech) simultaneously to the incubation with the adapter molecules.

Human Blood samples, in vitro transduction, flow cytometry and recombinant adapter molecules are as stated in Example 1. Activation was performed with a ratio of Dynabeads to T cells of 3 to 1. Adapter to knob ratio was of 32.5 to 1. Per cell a total of 100 genomic particles were used.

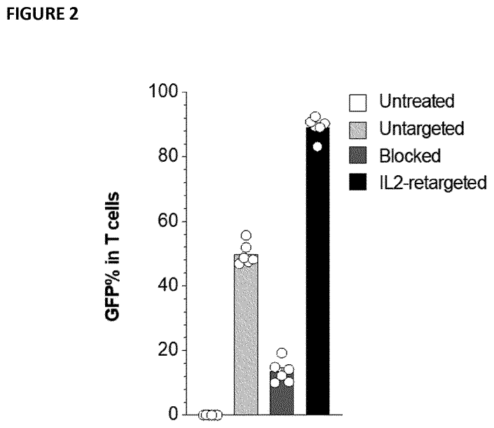

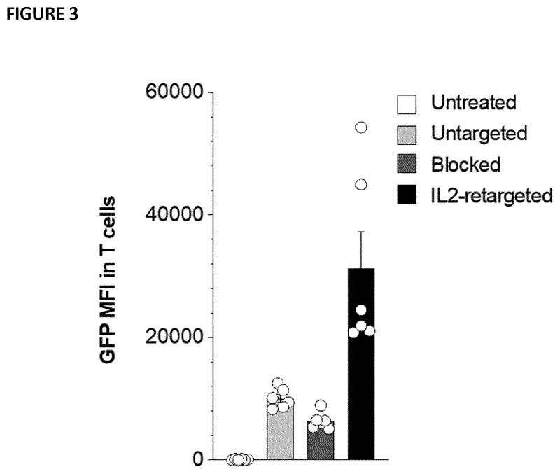

Measured was again GFP. FIG. 2 shows the percentage of T cells expressing GFP. FIG. 3 shows the mean fluorescence intensity (MFI). Compared to the unactivated T cells transduction efficacies were clearly higher. About 80% of T cells were effectively transfected with the IL-2 retargeted adapter. Also the untargeted virus like particles showed a transduction efficacy of about 50%. The blocked adapter showed a transfection of less than 20%. The same pattern is observed upon measurement of the absolute GFP expression levels.

### Example 4: Simultaneous Activation and Transduction is Critical for High Transduction Efficiency

In this experiment, it was investigated if the simultaneous activation of T cells is indeed responsible for the increase transduction efficacy. Three treatment groups were compared. In group A, T cells were activated with Dynabeads for 48 hours prior to the addition of adenoviruses displaying the IL-2 adapter. In group B, Dynabeads and adenoviruses were added simultaneously at time point zero. In group C, Dynabeads and adenoviruses were also added simultaneously at time point zero, with the addition of additional adenoviruses after 48 hours.

Human Blood samples, in vitro transduction, flow cytometry and recombinant adapter molecules are as stated in Example 1. Activation was performed with a ratio of Dynabeads to T cells of 3 to 1. Adapter to knob ratio was of 32.5 to 1. Per cell a total of 100 genomic particles were used.

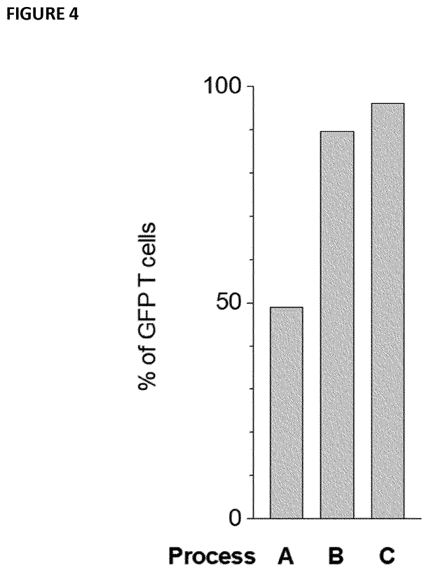

After 96 hours GFP was measured by flow cytometry. Results are shown in FIG. 4. Adding the IL2-retargeting adapter simultaneously to the activation of T cells significantly increase the transduction efficiency.

In a related experiment, four treatment groups were compared. In groups A and B, T cells were activated at time point zero with Dynabeads and recombinant IL-2. In both groups Dynabeads were removed after 48 hours. In group A additional recombinant IL-2 and viruses with the IL-2 retargeting adapter were added, whereas in group B only viruses with the IL-2 retargeting adapter were added.

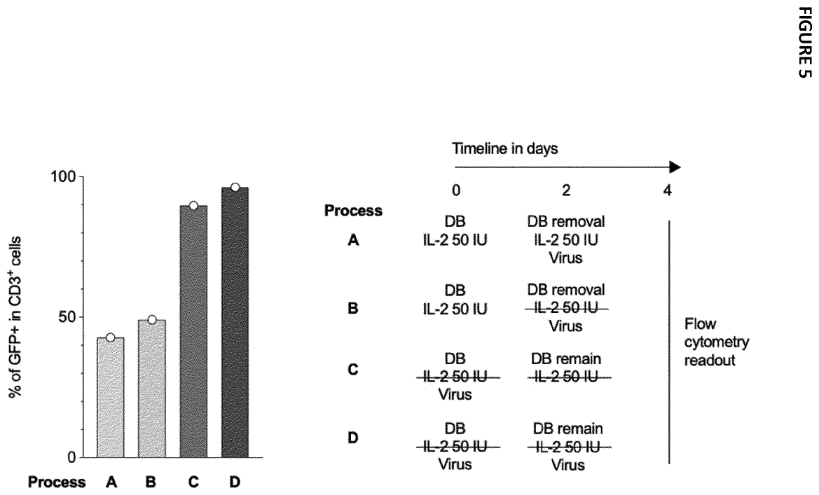

In groups C and D, Dynabeads and viruses with the IL-2 retargeting adapter were added at time point zero. In group D additional viruses were added after 48 hours. After 96 hours GFP was measured by flow cytometry. Results are shown in FIG. 5.

It was observed that the crucial parameter for efficient transduction of T cells is the simultaneous infection and activation of the T cells. Adding recombinant IL-2 instead of the viruses with the IL-2 retargeting adapter led to a less effective transduction.

### Example 5: Longer Incubation Times Increase Transduction Efficiency

In this experiment different incubations time were compared. PBMCs were activated and infected simultaneously for 16 and 48 hours as described above and without the addition of additional recombinant human IL-2. Compared were also viruses displaying the IL-2 adapter, to viruses displaying an anti-CD3 single chain antibody, an anti-CD28 single-chain antibody and a combination of the three aforementioned adapter molecules (1:1:1 ratio; adenovirus used at an MOI of 100 genomic particles per cell).

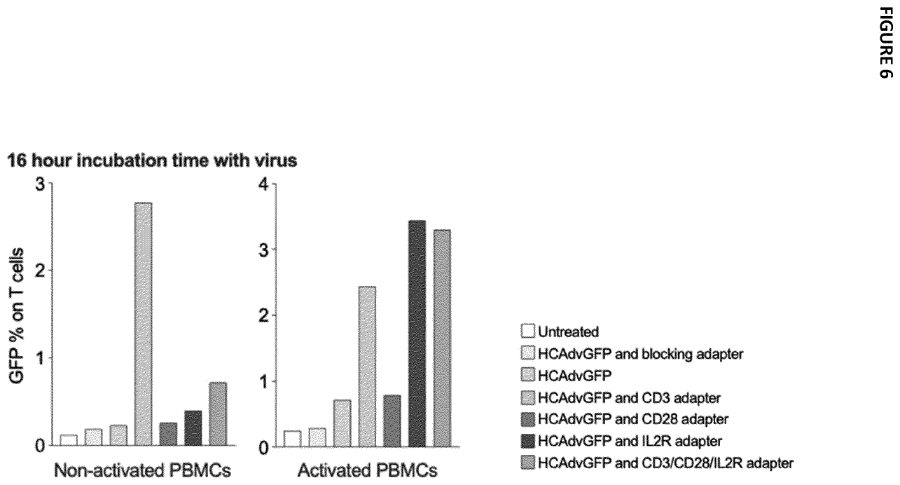

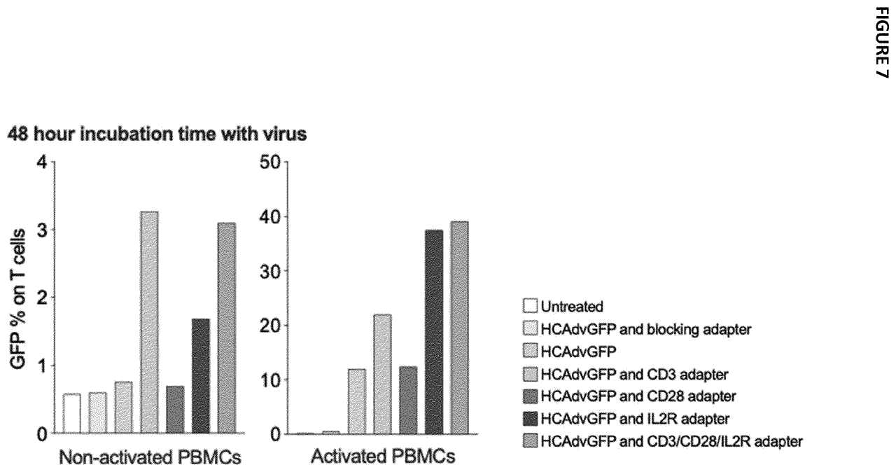

Results are shown in FIG. 6 (16 hours incubation) and FIG. 7 (48 hours incubation). Again GFP mas measured as described above. Incubation for 16 hours led to a transduction of less than 4% for both, non-activated and activated T cells. The same efficiency was observed at an incubation time of 48 hours for non-activated T cells. Strikingly, for activated T cells a significantly increased transduction efficacy was observed, which was most pronounces with adenoviruses displaying the IL-2 adapter. T cells displaying a combination of all three adapters showed about the same level of transduction. It was furthermore observed that the IL-2 adapter showed a higher transduction efficiency than the anti-CD3 single chain antibody adapter and the anti-CD28 single-chain antibody adapter.

In summary, it could be confirmed that a high transduction efficacy is dependent on adenoviruses displaying the IL-2 adapter. Furthermore, longer incubation times lead to a significant increase of the transduction efficacy.

### Example 6: PBMCs and T Cells are Transfected with Equivalent Transduction Efficiencies

In this experiment it was compared, if the effect of the IL-2 adapter is specific for T cells or if it can also be observed with PBMCs. Preparations of PBMCs and preparation of T cells were compared in the experimental set up described above. Incubation time was 48 hours.

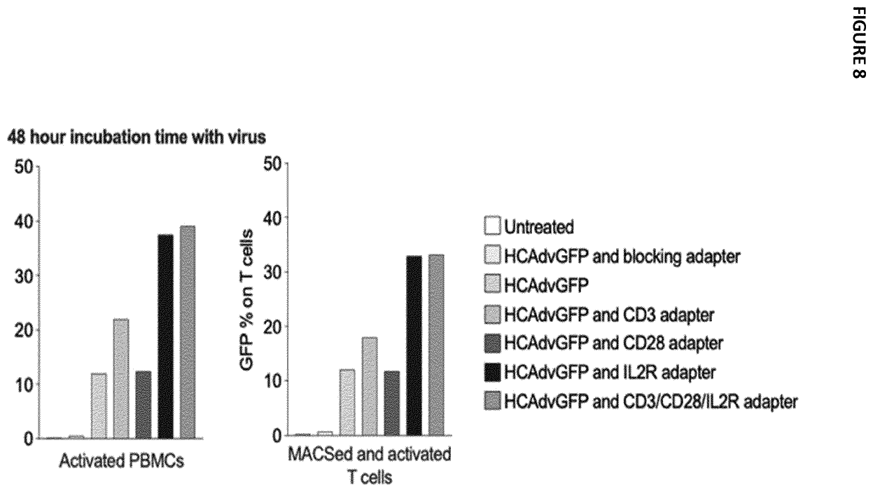

Results are shown in FIG. 8. The effect of the IL-2 adapter was confirmed for both cell populations (T cells and PBMCs). It hence makes no difference if T cells are infected directly of if PBMCs are used.

### Example 7: Transgenes are Detectable for at Least 2 Weeks after Transfection

In this experiment it was investigated how long the transgene could be detected in T cells transduced with adenoviruses displaying the IL-2 adapter. GFP expression was quantified by flow cytometry after infection. T cells were kept in ex vivo culture supplemented with new medium and cytokines to enable their expansion.

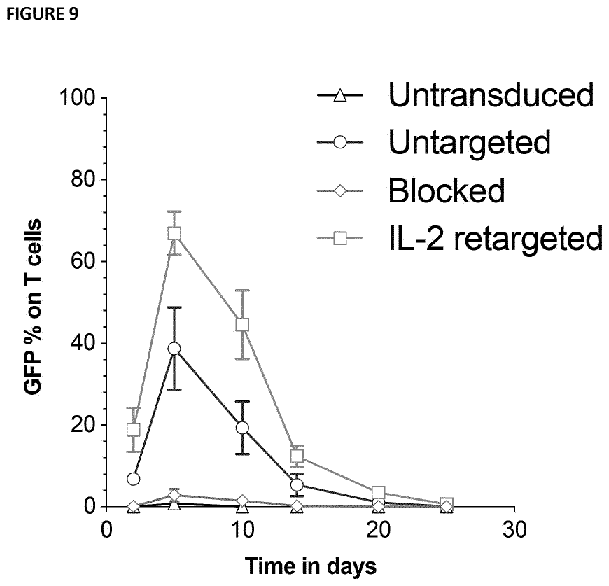

Results are shown in FIG. 9. GFP was readily detectable in cells transduced with adenoviruses displaying the IL-2 adapter. GFP remains detectable for at least two weeks after transduction.

### Example 8: Other Factors Only have Little Influence on Transduction Efficacy

In this experiment the Dynabeads-to-T cell ratio (3:1 and 1:1) and the adapter molecule-to-fiber knob ratio was varied (32.5:1 and 162.5:1). The percentage of T cells expressing GFP was measured after 5 days.

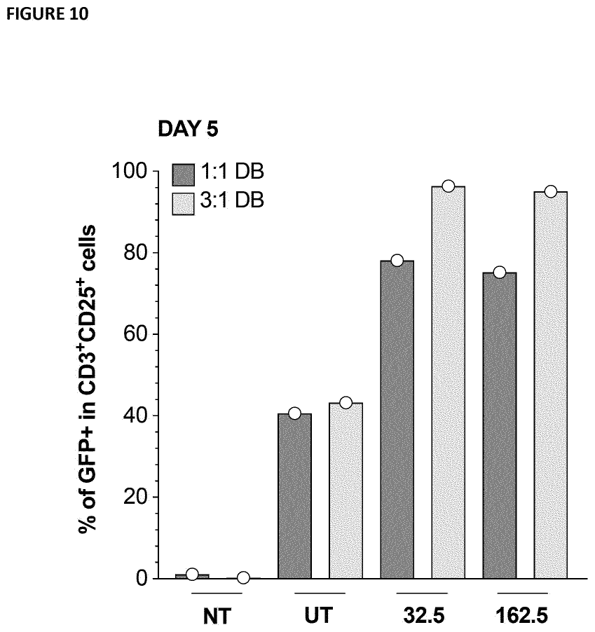

Results are shown in FIG. 10. It can be seen that a 3:1 of Dynabeads-to-T cell is better than a 1:1 ratio, although also the 1:1 ratio gives a satisfactory result. An increase of the adapter molecule-to-fiber knob ratio from 32.5:1 to 162.5:1 did not lead to an increase of transduction efficacy, i.e a 32.5:1 ratio is sufficient.

### Example 9: Transduction Efficiency on Simultaneously Activated Exceeds 95%

In this experiment, the transduction protocol optimized in the preceding experiments was tested with T cells from multiple donors. A Dynabeads-to-T cell ratio of 3:1, and an adapter-to-fiber knob ratio of 32.5:1 was used. The mixture was supplemented with 50 IU per ml of IL-2. Transfection was performed with 100 genomic particles per T cell.

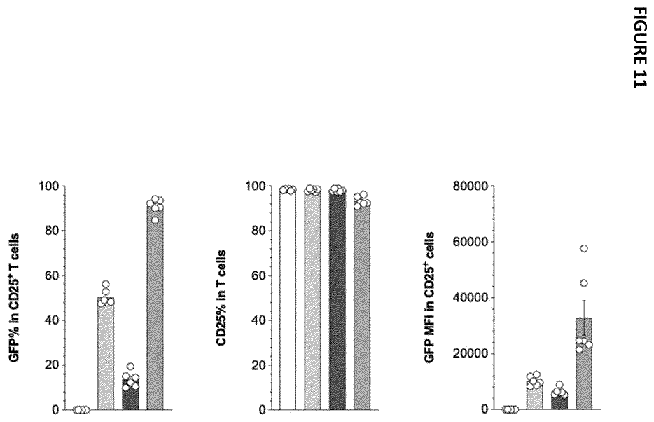

Results are shown in FIG. 11. A consistent transduction efficiency of above 95% can be achieved with these conditions. The number of activated T cells exceed 90% for all test conditions, but only T cells transduced with the IL-2 adapter showed a high expression of the GFP marker.

### Example 10: Transduction does not Impact T Cell Viability

In this experiment, the influence of the transduction process on the viability of the T cells was investigated. The number of genomic particles per cell and the adapters to fiber knobs was titrated with the experimental conditions of Example 9. The absolute number of T cells was determined by Flow cytometry using counting beads.

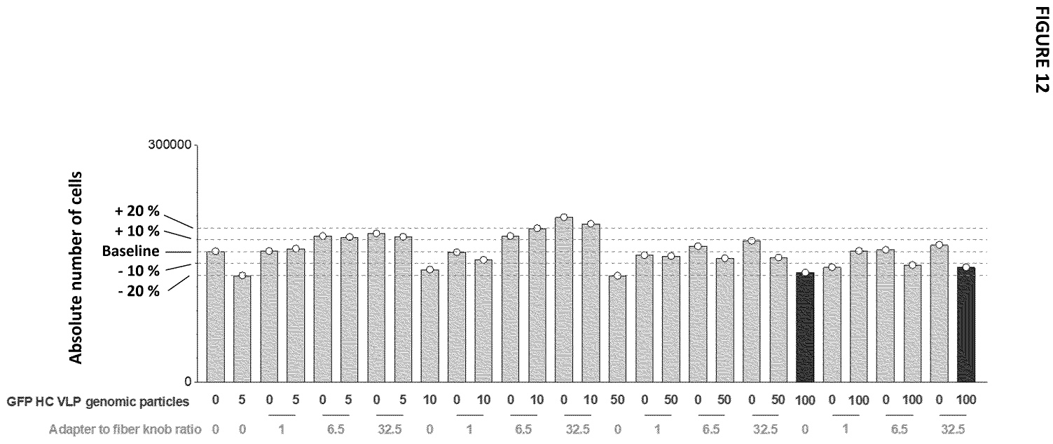

Results are shown in FIG. 12. It was found that the number of genomic particles and the adapters to fiber knobs ratio did not have a significant impact on T cell viability.

### Example 11: Neither the Virus, Nor the Adapter Skews the Cell Population Towards CD4 or CD8 T Cells

In this experiment it was tested if the adapter or the virus skews the cell population towards CD4-positive or CD8-positive T cells. To do so, T cells were differentiated for their expression of CD4 or CD8 using Flow cytometry.

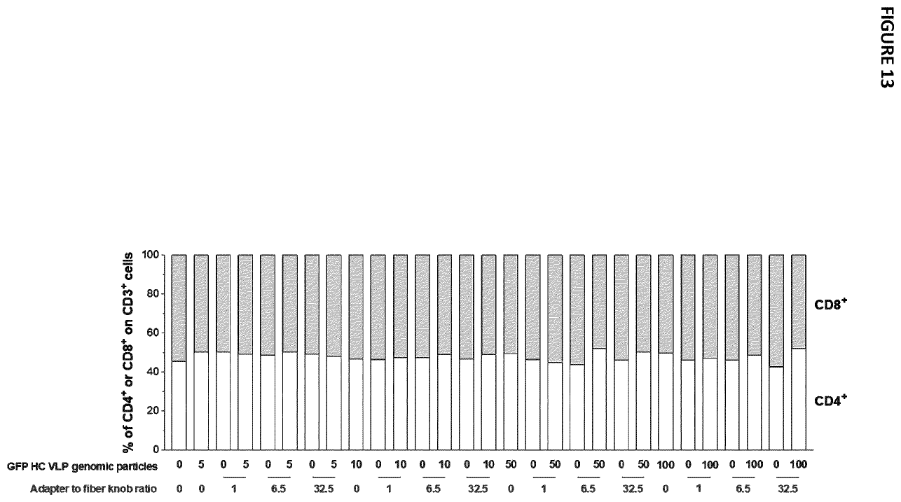

Results are shown in FIG. 13. It was observed that neither the adapter, nor the virus skews the cell population towards CD4-positive or CD8-positive T cells.

### Example 12: CD4-Positive T Cells are Preferably Transduced

In this experiment it was tested if CD4-positive or CD8 positive T cells are preferably transduced. The number of genomic particles per cell and the adapters to fiber knobs was titrated with the experimental conditions of Example 9. T cells were separated into a CD4-positive and a CD8-positive and transduction of the two cell populations was quantified.

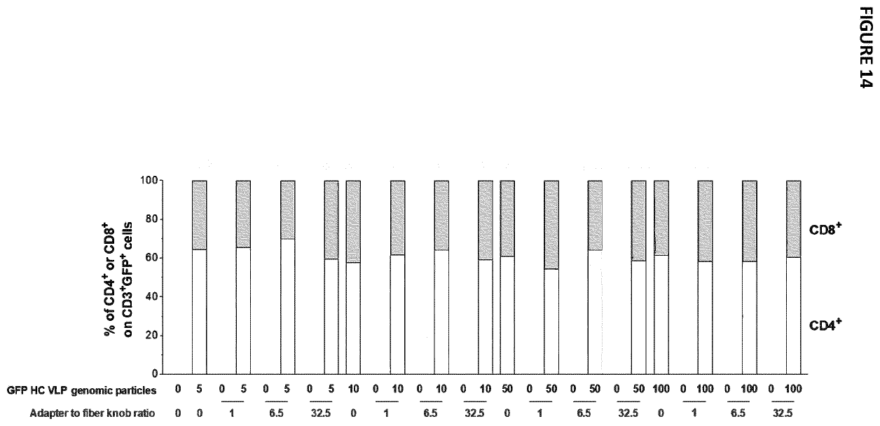

Results are shown in FIG. 14. Under all conditions tested, CD4-positive T cells were slightly better transduced as compared to CD8-positive T cells.

### Example 13: Neither the Virus, Nor the Adapter Skews the Cell Population Towards Conventional T Cells or Regulatory T Cells

In this experiment it was tested if the adapter or the virus skews the cell population towards conventional T cells (Tconv) or regulatory T cells (Treg). To do so, T cells were separated into Tconv and a Treg population using Flow cytometry where after gating for CD4 positive cells, Treg cells were defined as the {CD25^{high} and CD127^{low}} population and the Tconv were defined as not {CD25^{high} and CD127^{low}}.

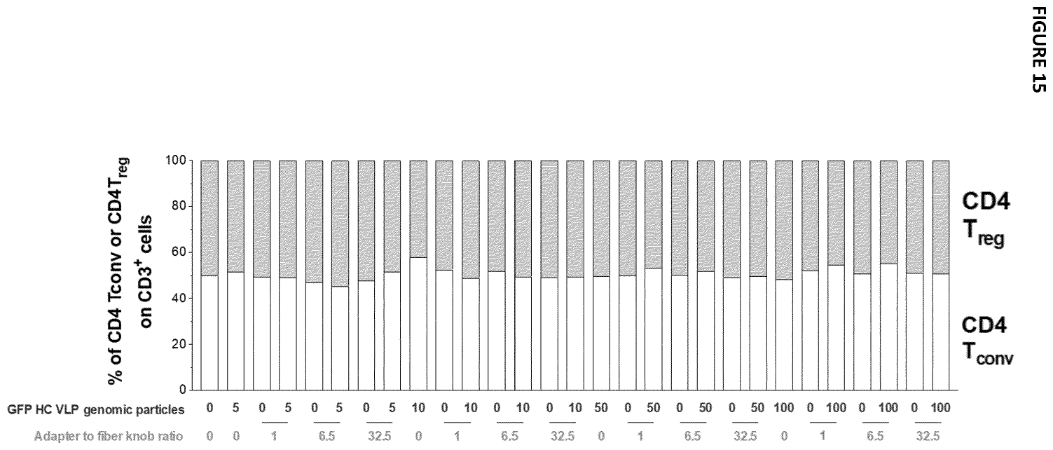

Results are shown in FIG. 15. It was observed that neither the adapter, nor the virus skews the cell population towards conventional T cells or regulatory T cells.

### Example 14: Naïve and Central Memory are the Predominant Compartments after T Cell Transduction

In this experiment it was tested if the experimental procedure preferentially directs the transduced cell towards specific effector or memory phenotypes. To do so, T cells were analyzed by flow cytometry and defined as Naïve {CCR7+CD45RA+}, Effector {CCR7−CD45RA+}, Effector memory {CCR7−CD45RA−} or Central memory {CCR7+CD45RA−}.

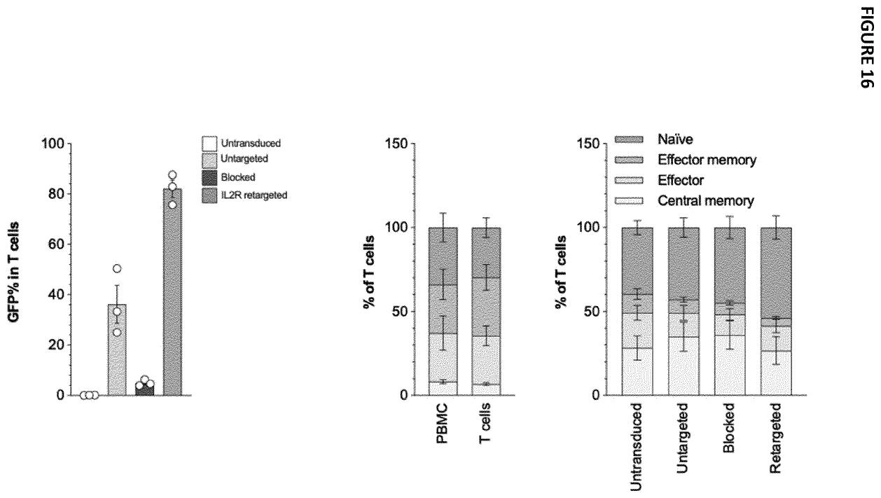

Results are shown in FIG. 16. It could be found that the experimental conditions spare the naïve and the central memory compartments. Adapter and virus do not influence the compartment. The activation step seems to be the most critical one.

### Example 15: High-Efficient Transduction of T Regulatory Cells Using IL-2-Adapter Retargeted Virus Like Particles

In this experiment, the IL-2 adapter was tested for its efficacy to transduce activated T regulatory cells. As described in Example 1, Treg cells were isolated from PBMCs. As described in Example 3, Treg cells were activated with Dynabeads (Human T-Expander CD3/CD28; ThermoFisher) and human recombinant human IL-2 (50 IU per ml; Miltenyi Biotech) simultaneously to the incubation with the adapter molecules.

Human Blood samples, in vitro transduction, flow cytometry and recombinant adapter molecules are as stated in Example 1. Activation was performed with a ratio of Dynabeads to T cells of 3 to 1. Adapter to knob ratio was of 32.5 to 1. Per cell a total of 100 genomic particles were used.

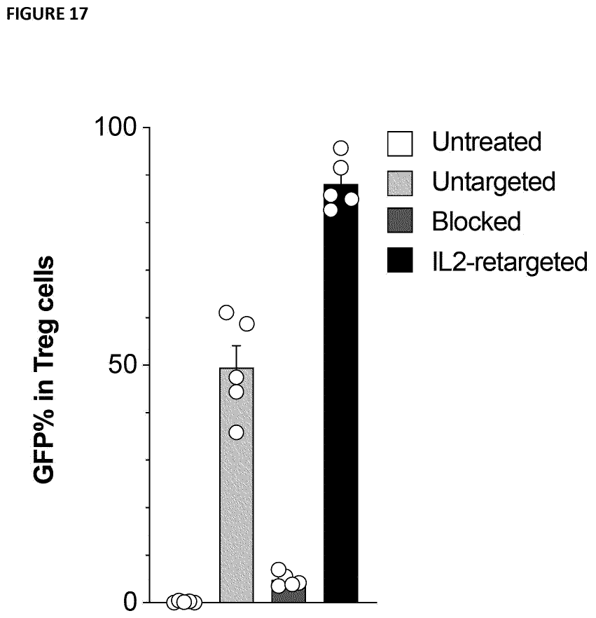

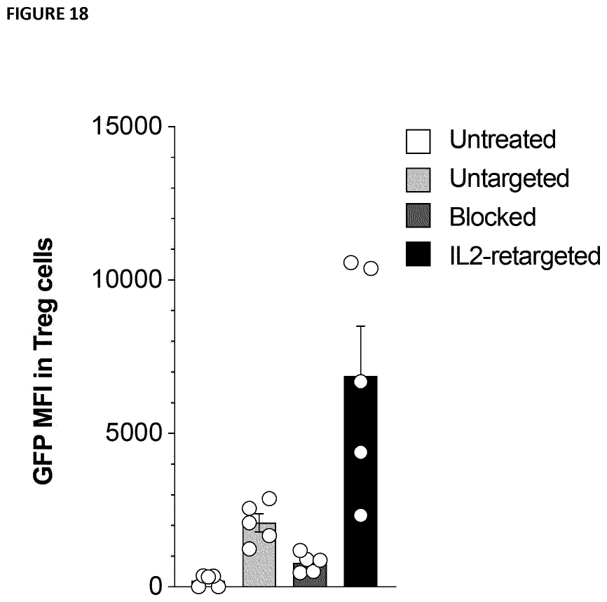

Measured was again GFP. FIG. 17 shows the percentage of Treg cells expressing GFP. FIG. 18 shows the mean fluorescence intensity (MFI). Like conventional T cells, also Treg cells were effectively transduced with the IL-2 retargeted adapter around 90%, as compared to about 55% with the untargeted adapter. The blocked adapter showed a transfection of less than 10%. The same pattern is observed upon measurement of the absolute GFP expression levels.

### Example 16: Conventional Adenoviruses have a Limited Transduction Efficiency for T Cells

In this experiment, conventional first-generation adenoviruses were tested for their transduction efficiency towards T cells. The transduction efficiency of an untargeted adenoviral vector, i.e. an adenoviral vectors not carrying any adapter, was compared to targeted adenoviruses, i.e. adenoviruses displaying different targeting moieties, e.g. a DARPin specific for CD4. As a control a blocked adenovirus was used, i.e. an adenovirus carrying an E2_5 adapter as a targeting moiety. The E2_5 adapter does not have specificity for any known antigen.

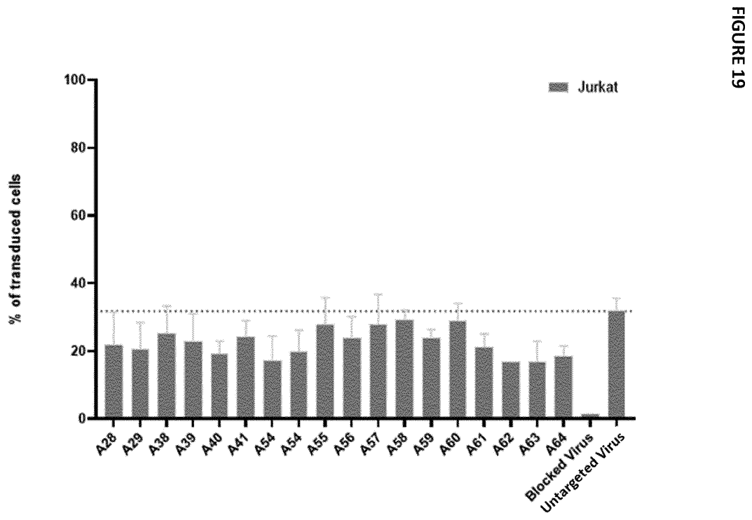

Exemplary results of the transduction efficiency of Jurkat cells are shown in FIG. 19 for anti-CD4 DARPins (see e.g. PLOS Pathogens (2008) 4: e1000109). None of the tested constructs showed a transduction efficiency towards Jurkat cells that exceeded the transduction efficiency of an untargeted adenovirus. Blocked adenovirus, containing the E2_5 adapter were essentially devoid of any transduction.

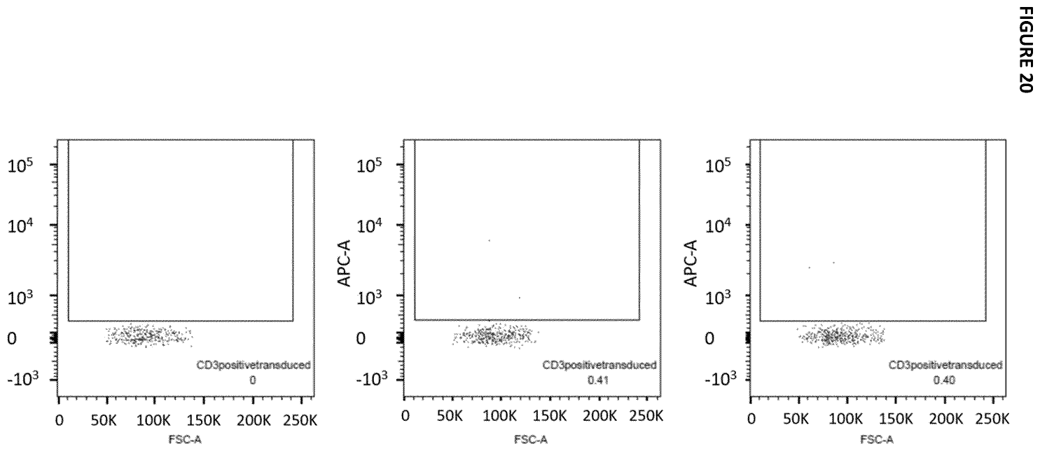

A similar experiment was performed with untargeted adenoviruses encoding the marker protein iRFP670 and primary, unactivated T cells. Cells were analyzed by flow cytometry. Results are shown in FIG. 20 for three independently prepared primary T cell populations. Essentially no transduction could be seen in any of the experiments.

These results demonstrate that conventional adenoviruses have a very limited transduction efficiency in T cells.

### Example 17: Combination of Adapters

As has been shown in Example 16, conventional adenoviruses have a very limited transduction efficiency in T cells if they are not simultaneously activated, which is also unaffected by the single adapter molecules tested. It was hypothesized that adenoviruses expressing more than one targeting moiety or adapter could have a beneficial effect on transduction efficiency.

Therefore, adenoviruses were generated that display one, two or all three of the following entities:
an anti-CD3 single chain antibody (scFv),
an anti-CD28 single chain antibody (scFv),
an interleukin 2 polypeptide.

Each of these entities was displayed via the adapter technology described above. The specific adapters have the following amino acid sequence:

| | | |
|---|---|---|
| SEQ ID No. | Description | Sequence |

Anti-CD3 adapter

| | | |
|---|---|---|
| 8 | Whole | GPGSDIQMTQSPSSLSASVGDRVTITCRASQDIRNYLNWYQQKPGKAPKLLIYYTSR |
| | molecule | LESGVPSRFSGSGSGTDYTLTISSLQPEDFATYYCQQGNTLPWTFGQGTKVEIKRGG |
| | | GGSGGGGSGGGGSEVQLVESGGGLVQPGGSLRLSCAASGYSFTGYTMNWVRQA |
| | | PGKGLEWVALINPYKGVSTYNQKFKDRFTISVDKSKNTAYLQMNSLRAEDTAVYYC |
| | | ARSGYYGDSDWYFDVWGQGTLVTVSSKLGGGGSGGGGSGGGGSGGGGSRSDLG |
| | | KKLLEAARAGQDDEVRILMANGADANAYDHYGRTPLHMAAAVGHLEIVEVLLRNG |
| | | ADVNAVDTNGTTPLHLAASLGHLEIVEVLLKYGADVNAKDATGITPLYLAAYWGHL |
| | | EIVEVLLKHGADVNAQDKFGKTPFDLAIDNGNEDIAEVLQGVRIFAGNDPAHTATG |
| | | SSGISSPTPALTPLMLDEATGKLVVWDGQKAGSAVGILVLPLEGTETALTYYKSGTFA |
| | | TEAIHWPESVDEHKKANAFAGSALSHAALP |
| 11 | Anti-CD3 part | GPGSDIQMTQSPSSLSASVGDRVTITCRASQDIRNYLNWYQQKPGKAPKLLIYYTSR |
| | | LESGVPSRFSGSGSGTDYTLTISSLQPEDFATYYCQQGNTLPWTFGQGTKVEIKRGG |
| | | GGSGGGGSGGGGSEVQLVESGGGLVQPGGSLRLSCAASGYSFTGYTMNWVRQA |
| | | PGKGLEWVALINPYKGVSTYNQKFKDRFTISVDKSKNTAYLQMNSLRAEDTAVYYC |
| | | ARSGYYGDSDWYFDVWGQGTLVTVSSKL |
| 12 | CD3-HCDR1 | GYTMN |
| 13 | CD3-HCDR2 | LINPYKGVSTYNQKFKD |
| 14 | CD3-HCDR3 | SGYYGDSDWYFDV |
| 15 | CD3-LCDR1 | RASQDIRNYLN |
| 16 | CD3-LCDR2 | YTSRLES |
| 17 | CD3-LCDR3 | QQGNTLPWT |
| 35 | Linker | GGGGSGGGGSGGGGSGGGGSRS |
| 36 | Adapter part | DLGKKLLEAARAGQDDEVRILMANGADANAYDHYGRTPLHMAAAVGHLEIVEVLL |
| | | RNGADVNAVDTNGTTPLHLAASLGHLEIVEVLLKYGADVNAKDATGITPLYLAAYW |
| | | GHLEIVEVLLKHGADVNAQDKFGKTPFDLAIDNGNEDIAEVLQGVRIFAGNDPAHT |
| | | ATGSSGISSPTPALTPLMLDEATGKLVVWDGQKAGSAVGILVLPLEGTETALTYYKS |
| | | GTFATEAIHWPESVDEHKKANAFAGSALSHAALP |

Anti-CD28 adapter

| | | |
|---|---|---|
| 9 | Whole | GPGSDIQMTQSPSSLSASVGDRVTITCHASQNIYVWLNWYQQKPGKAPKLLIYKAS |
| | molecule | NLHTGVPSRFSGSGSGTDFTLTISSLQPEDFATYYCQQGQTYPYTFGGGTKVEIKRG |
| | | GGGSGGGGSGGGGSQVQLVQSGAEVKKPGASVKVSCKASGYTFTSYYIHWVRQA |
| | | PGQGLEWIGCIYPGNVNTNYNEKFKDRATLTVDTSISTAYMELSRLRSDDTAVYFCT |
| | | RSHYGLDWNFDVWGQGTTVTVSSKLGGGGSGGGGSGGGGSGGGGSRSDLGKKL |
| | | LEAARAGQDDEVRILMANGADANAYDHYGRTPLHMAAAVGHLEIVEVLLRNGAD |
| | | VNAVDTNGTTPLHLAASLGHLEIVEVLLKYGADVNAKDATGITPLYLAAYWGHLEIV |
| | | EVLLKHGADVNAQDKFGKTPFDLAIDNGNEDIAEVLQGVRIFAGNDPAHTATGSSG |
| | | ISSPTPALTPLMLDEATGKLVVWDGQKAGSAVGILVLPLEGTETALTYYKSGTFATEA |
| | | IHWPESVDEHKKANAFAGSALSHAALP |
| 18 | Anti-CD28 part | GPGSDIQMTQSPSSLSASVGDRVTITCHASQNIYVWLNWYQQKPGKAPKLLIYKAS |
| | | NLHTGVPSRFSGSGSGTDFTLTISSLQPEDFATYYCQQGQTYPYTFGGGTKVEIKRG |
| | | GGGSGGGGSGGGGSQVQLVQSGAEVKKPGASVKVSCKASGYTFTSYYIHWVRQA |
| | | PGQGLEWIGCIYPGNVNTNYNEKFKDRATLTVDTSISTAYMELSRLRSDDTAVYFCT |
| | | RSHYGLDWNFDVWGQGTTVTVSSKL |
| 19 | CD28-HCDR1 | SYYIH |
| 20 | CD28-HCDR2 | CIYPGNVNTNYNEKFKD |
| 21 | CD28-HCDR3 | SHYGLDWNFDV |
| 22 | CD28-LCDR1 | HASQNIYVWLN |
| 23 | CD28-LCDR2 | KASNLHT |
| 24 | CD28-LCDR3 | QQGQTYPYT |
| 35 | Linker | GGGGSGGGGSGGGGSGGGGSRS |
| 36 | Adapter part | DLGKKLLEAARAGQDDEVRILMANGADANAYDHYGRTPLHMAAAVGHLEIVEVLL |
| | | RNGADVNAVDTNGTTPLHLAASLGHLEIVEVLLKYGADVNAKDATGITPLYLAAYW |
| | | GHLEIVEVLLKHGADVNAQDKFGKTPFDLAIDNGNEDIAEVLQGVRIFAGNDPAHT |
| | | ATGSSGISSPTPALTPLMLDEATGKLVVWDGQKAGSAVGILVLPLEGTETALTYYKS |
| | | GTFATEAIHWPESVDEHKKANAFAGSALSHAALP |

IL-2 adapter

| | | |
|---|---|---|
| 6 | Whole | GPGSAPTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCEYADETA |
| | molecule | TIVEFLNRWITFCQSIISTLTKLGGGGGGGGSGGGGSGGGGSRSDLGKKLLEAARA |
| | | GQDDEVRILMANGADANAYDHYGRTPLHMAAAVGHLEIVEVLLRNGADVNAVDT |
| | | NGTTPLHLAASLGHLEIVEVLLKYGADVNAKDATGITPLYLAAYWGHLEIVEVLLKHG |
| | | ADVNAQDKFGKTPFDLAIDNGNEDIAEVLQGVRIFAGNDPAHTATGSSGISSPTPAL |
| | | TPLMLDEATGKLVVWDGQKAGSAVGILVLPLEGTETALTYYKSGTFATEAIHWPESV |
| | | DEHKKANAFAGSALSHAALP |
| 25 | IL-2 region | GPGSAPTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCEYADETA |
| | | TIVEFLNRWITFCQSIISTLTKL |
| 35 | Linker | GGGGSGGGGSGGGGSGGGGSRS |
| 36 | Adapter part | DLGKKLLEAARAGQDDEVRILMANGADANAYDHYGRTPLHMAAAVGHLEIVEVLL |
| | | RNGADVNAVDTNGTTPLHLAASLGHLEIVEVLLKYGADVNAKDATGITPLYLAAYW |
| | | GHLEIVEVLLKHGADVNAQDKFGKTPFDLAIDNGNEDIAEVLQGVRIFAGNDPAHT |
| | | ATGSSGISSPTPALTPLMLDEATGKLVVWDGQKAGSAVGILVLPLEGTETALTYYKS |
| | | GTFATEAIHWPESVDEHKKANAFAGSALSHAALP |

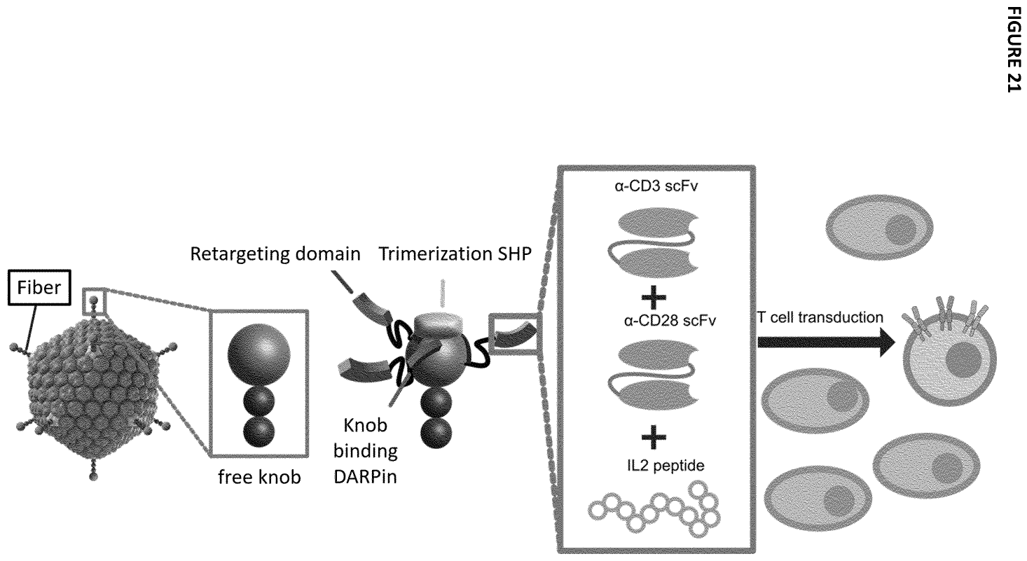

The overall strategy is depicted in FIG. 21.

### Example 18: A Combination of Adapters Leads to a Strong Increase of Transduction of T Cells

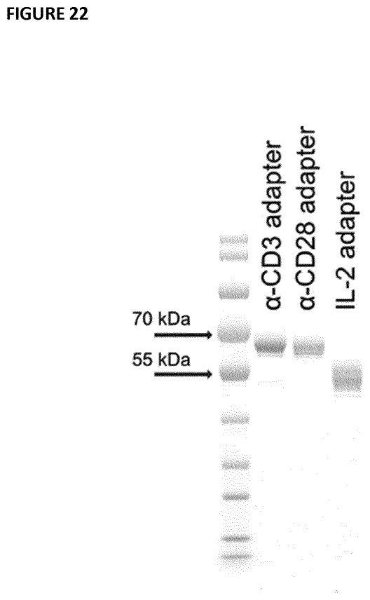

The three adapter molecules described above were purified. FIG. 22 shows an SDS-PAGE confirming the purity of the adapters.

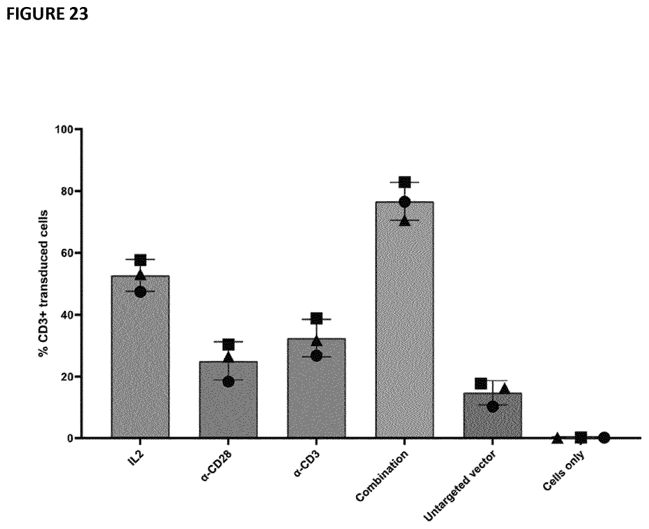

Adenoviral vectors were mixed with an equal ratio of the three different adapter molecules, i.e. the anti-CD3 adapter, the anti-CD28 adapter and the interleukin 2 adapter. Adenoviruses contain 12 knobs to which the adapters molecules can bind. Since binding of the adapters follows a Gaussian distribution, it can be assumed that about 97.7% of all adenoviruses display at least one adapter molecule of each type. As control, adenoviruses displaying only one of the three adapters as well as untargeted vectors were used. Adenoviruses (with an MOI of 20,000 virus particles/cell) were then used to transfect primary T cells pre-activated with dynabeads (Dynabeads Human T-Expander CD3/CD28 (ThermoFisher)) prior to the transfection, rather than simultaneously as described prior. Transfection was measured by flow cytometry by expression of reporter protein. Results are shown in FIG. 23.

It can be seen that a combination of the adapters leads to a strongly increased transduction efficiency as compared to adenoviruses displaying only one type of adapters.

### Example 19: Activation of T Cells is Important for Transduction and Combination of the Adapters Leads to Activation

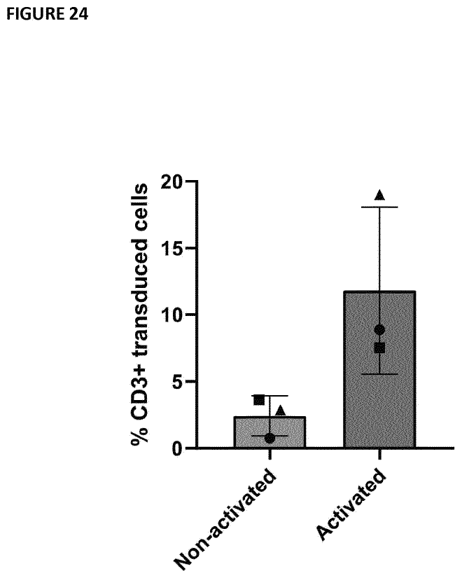

Unactivated primary T cells and T cells activated with dynabeads (Dynabeads Human T-Expander CD3/CD28 (ThermoFisher)) were transfected with untargeted adenoviral vectors, i.e. adenoviruses not displaying any adapters, to investigate the importance of T cell activation prior to transduction. Measured was reporter protein expression of iRFP670 in CD3+ cells via an anti-CD3 antibody conjugated to a fluorophore (Alexa-488). As can be seen in FIG. 24, activation is important for transduction efficiency.

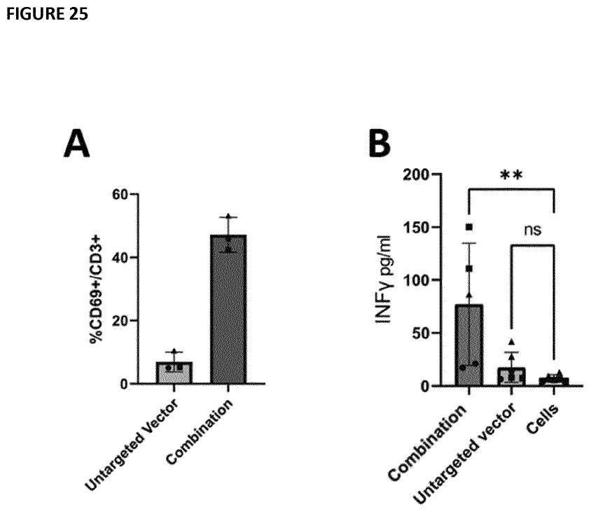

Next, unactivated primary T cells were transfected with adenoviruses displaying a combination of the adapter molecules (with an MOI of 20,000 virus particles/cell, as determined by the nanodrop), i.e. an adapter comprising an antigen-binding moiety specifically binding to CD3, an adapter comprising an antigen-binding moiety specifically binding to CD28, and an adapter comprising a functional interleukin 2 polypeptide, in comparison to untargeted adenoviruses. Also interferon γ secretion was measured in the transfected cells as a marker of T cell activation. Results of transduction efficiency are shown in FIG. 25, panel A, for interferon γ release in FIG. 25, panel B. It can be seen that the combination of the adapter molecules not only leads to a strongly increased transduction efficiency, but also to an activation of T cells, as measured by interferon γ release. The combination of the adapter molecules of the present disclosure hence leads to an activation of T cells.

### Example 20: Investigation of Various Ratios of the Adapter Molecules

As has been shown above, adenoviruses displaying the combination of adapter molecules of the present disclosure are capable of transducing T cells. Next it was explored if the ratio of the three adapter molecules has an influence on transduction efficiency. Adapter molecules were mixed at various ratios before being incubated with the adenoviruses (with an MOI of 20,000 virus particles/cell).

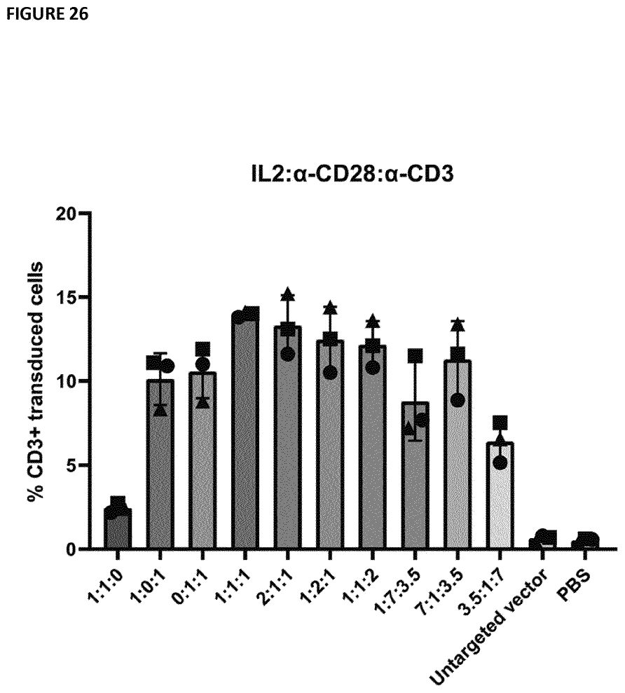

Results are show in FIG. 26. Adapter molecules at a 1:1:1 ratio showed the highest transduction efficiency. A 2:1:1 ratio, irrespective of which adapter was added with a higher concentration, showed a slightly decreased, but still high transduction efficiency. Even if one of the adapters was present at a lower concentration compared to the other two adapters (7:3,5:1 ratio), transduction efficiencies were still high. Even if one of the adapters was completely absent, transduction efficiencies were still high, provided the other two adapters are present. Exception was the anti-CD3 adapter, the absence of which strongly decrease transduction efficiencies. In summary, it could be demonstrated that a combination of the tested adapters leads to a strongly increased transduction efficiency when displayed on adenoviruses.

### Example 21: Compatibility of the System with High-Capacity Adenoviruses

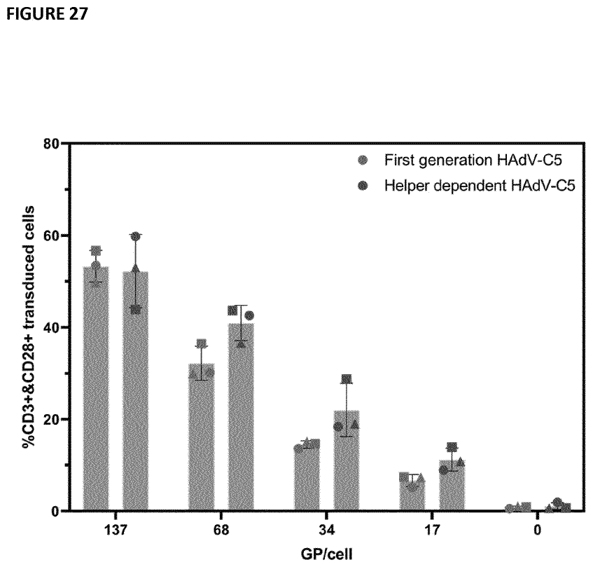

After demonstrating T cell-directed transduction by utilizing a combination of CD3, CD28, and IL2-R targeted adapters, we investigated compatibility with high-capacity adenoviral vectors (HC-AdV) enabling the full potential of adenoviral vectors. A major benefit of adenoviral vectors lies in HC-AdV vectors, packaging up to 36 kb while lacking all viral genes, excluding residual expression of viral proteins and recognition through MHC I presentation. To verify the compatibility of our adapters we generated HC-HAdV and compared the transduction efficiency to a first generation (FG) HAdV-C5. Calculations of adapter: knob ratios were performed using optically determined viral particles. However, to achieve comparable multiplicity of infection (MOI), genomic particles/cell (GP/cell) were used for titrations. Results are depicted in FIG. 27, demonstrating that the transduction efficiency of the combination of the adapter molecules is compatible with high-capacity adenoviruses

### Example 22: Adapter-Mediated Transduction is Specific and can be Oversaturated

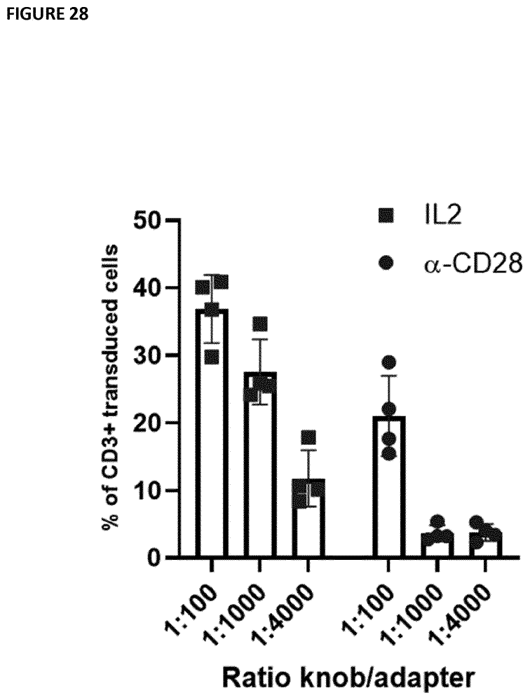

Blocking adenoviral vectors with E2_5 drastically reduced transduction efficiency (data not shown). The specificity of the targeted receptors was also indirectly confirmed by altering the ratio between number of the adenoviruses (i.e. the number of knobs to be precise; each adenovirus carries twelve knobs) and the number of the adapter molecules. This was tested with the anti-CD28 adapter and the interleukin 2 adapter. Results are shown in FIG. 28.

Increasing the number of adapter molecules over a certain threshold leads to a decrease in transduction efficiency, indicating that the system is oversaturated and transduction is receptor specific.

### Example 23: Transduction is CD3 Specific in Jurkat Cells

To test if transduction of T cell is CD3 specific we made use of Jurkat cells that are knocked-out in CD3. Successful CD3 knock-out was confirmed by FACS.A 1:1 mixture of wild-type Jurkat cells and Jurkat cells with a CD3 knock out were transfected with adenoviruses displaying the combination of adapter molecules of the present disclosures. The transduced cells were analyzed by FACS. As a control, adenoviruses displaying an untargeted vector was used.

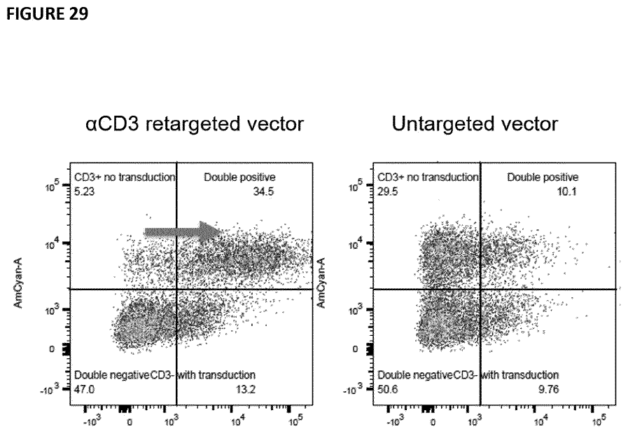

Results are shown in FIG. 29. The cell population expressing CD3 (i.e. wild-type Jurkat cells) exhibited a strong increase in transduction efficiency when transduced with adenoviral vectors displaying a recombinant protein comprising an anti-CD3 binding moiety. CD3-negative Jurkat cells hardly showed any change in transduction efficiency. Also, Jurkat cells transduced with an untargeted vector did not show an increase of transduction efficiency.

### Example 24: Transduction is Also CD3 Specific in Primary Cells

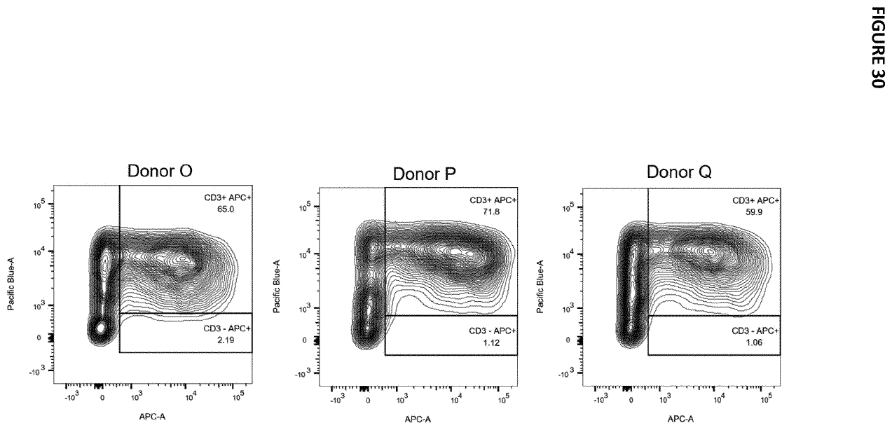

A similar experiment was performed with PBMCs. PBMCs were transduced with adenoviruses displaying the combination of vectors of the present disclosure (1:1:1 ratio). Results are shown in FIG. 30 for three different PBMC populations from different donors. Cell populations with a CD3+ phenotype show a clearly increased transduction frequency as measured reporter protein expression via flow cytometry. The same result was also achieved with shielded adenoviruses displaying the combination of vectors of the present disclosure (data not shown).

### Example 25: Transduced T Cells Show No Toxic Overactivation

It was also examined if PBMCs transduced with adenoviruses displaying the combination of adapters of the present disclosure show a toxic overreaction. Samples of PBMCs transduced with adenoviruses displaying the combination of adapters of the present disclosure were collected every 24 hours post transduction and cell numbers were determined.

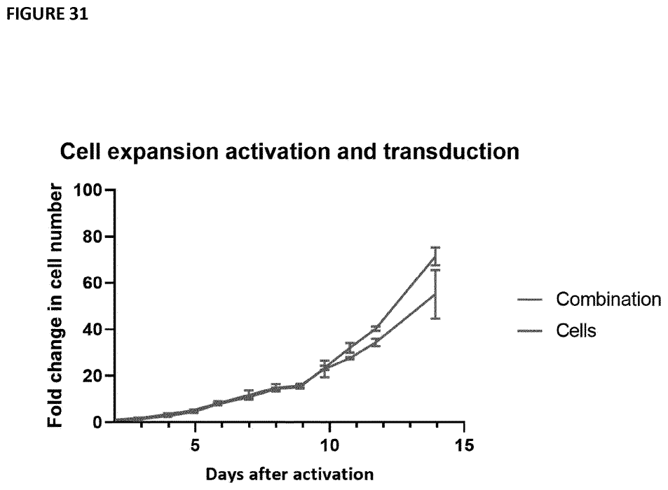

Results are shown in FIG. 31. Compared to untransduced PBMCs, the cell count of transduced cells was similar, thereby confirming that transduction does not lead to an overactivation of cells. Cell counts were also similar for PBMCs transduced with the three individual adapters, as well as a non-binding adapter and the untargeted adenovirus (data not shown).

### Example 26: In Vivo Transduction of Activated T Cells in Immunodeficient Mice

In this study NSG mice were used. NSG do not have an own immune system and do not carry any T cells. 4×10⁶ activated human PBMCs and 8×10¹⁰ retargeted viral particles were injected to the mice intraperitoneally. Control mice were transduced with untargeted vector or were injected with PBMCs only. After 48 h the T cell population was analyzed. Experiments were repeated twice in two separate studies with two separate donors

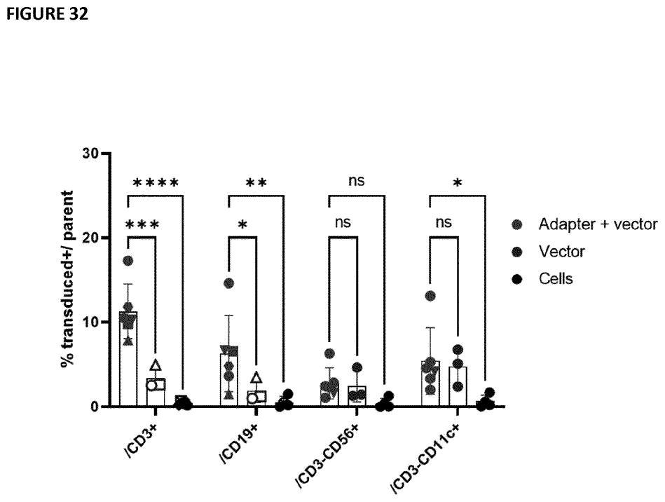

First, the retrieved cell population was analyzed, to see which cell type was preferentially transduced. Results are shown in FIG. 32. T cells, indicated by the CD3+ phenotype, were by far the cell population that was most strongly transduced. A minor level of transduction was seen in B cells (CD19+ phenotype. Essentially no transduction was seen in NK cells (CD56+) and monocytes/DCs (CD11c+).

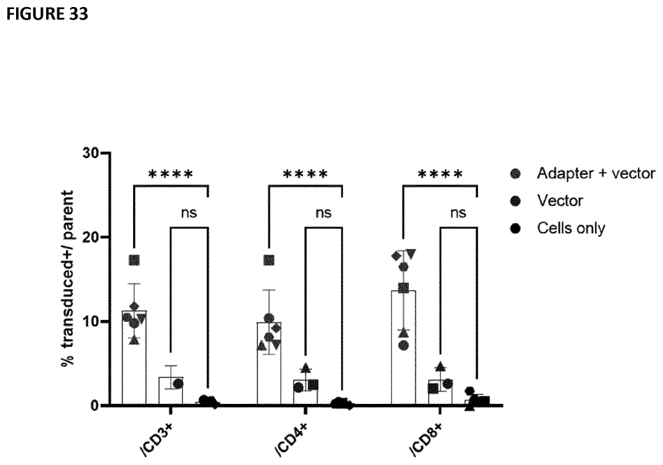

Next, the T cell population was analyzed in more detail. T cells were separated into T helper cells (CD4+) and cytotoxic T cells (CD8+). Results are shown in FIG. 33 (left: all T cell; middle: T helper cells; right; cytotoxic T cells). It could be demonstrated that all T cell types, T helper cells and cytotoxic T cells, were transduced by adenoviruses displaying a combination of the adapters of the present disclosure.

### Example 27: Also Non-Activated T Cells can be Transduced in Immunodeficient Mice In Vivo

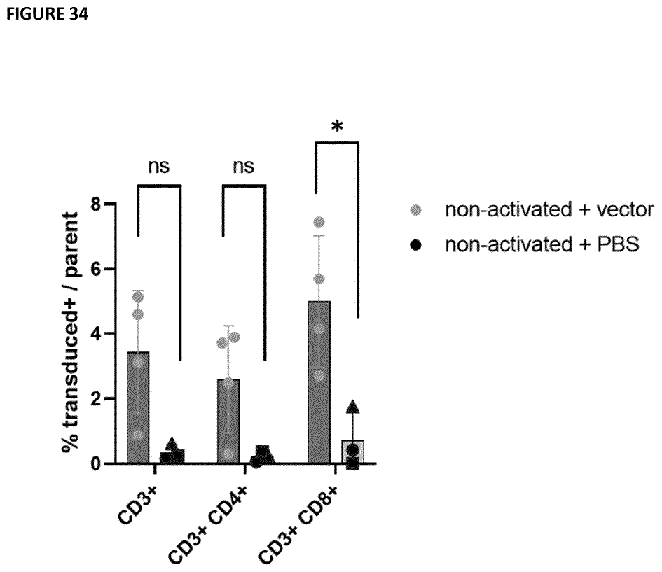

A similar experiment was performed as in Example 26. This time however, PBMCs were not activated prior to injection into mice. Results are shown in FIG. 34. Although transduction efficiency of unactivated T cells was lower as compared to activated T cells, transduction was possible. Again, both types of T cells could be transfected-T helper cells and cytotoxic T cells. Transduction efficiencies were slightly higher for cytotoxic T cells.

### Example 28: In Vivo Transduction of Non-Activated T Cells in Reconstituted Mice

In this study reconstituted NSG mice (NOD/LtSz-scid IL2Rγnull mice) were used. NSG mice were gamma-irritated between day 1-5 and injected with 1-3×10⁵ CD34+ from human fetal liver (HFL) into the mice liver. Reconstituted NSG mice have functional human T-, NK- and B cells, macrophages as well as DCs. The ratio and the surface expression pattern can, however, vary from humans. 1×10¹¹ retargeted vectors were injected to the mice intravenously. Control mice were injected with PBS. Experiments were repeated twice in two independent studies with two independent donors for reconstitution.

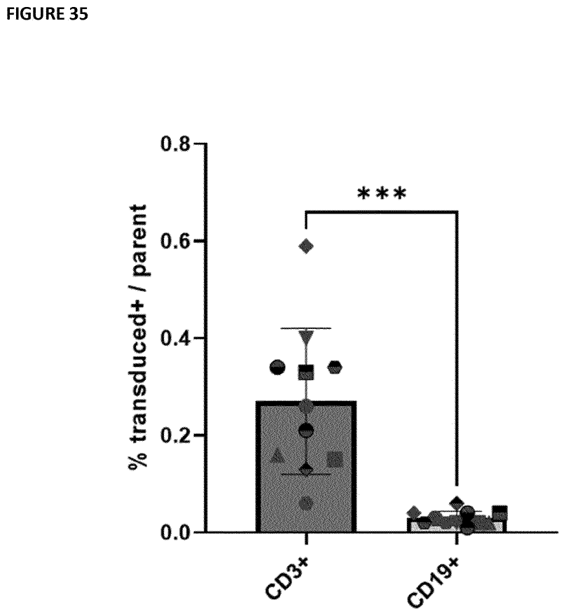

48 h after intravenous (I.v.) injection of 1×10¹¹ retargeted viral particles (n=11) or PBS (n=5), the spleen and blood were harvested and analyzed by flow cytometry. Results are shown in FIG. 35. T cells, indicated by the CD3+ phenotype, showed a significantly higher transduction efficiency compared to B cells (CD19+ phenotype).

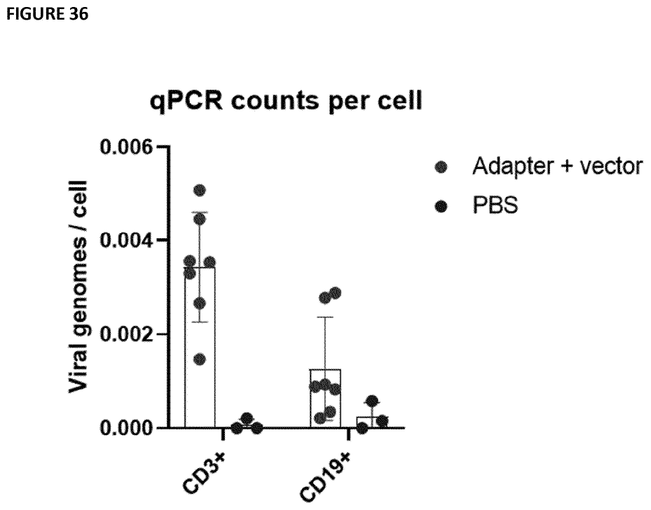

Next, results were confirmed by quantitative PCR (qPCR). Results are shown in FIG. 36. Again, T cells, indicated by the CD3+ phenotype, showed a significantly higher transduction efficiency compared to B cells (CD19+ phenotype).

### Example 29: Transduction of Pre-Activated T Cells

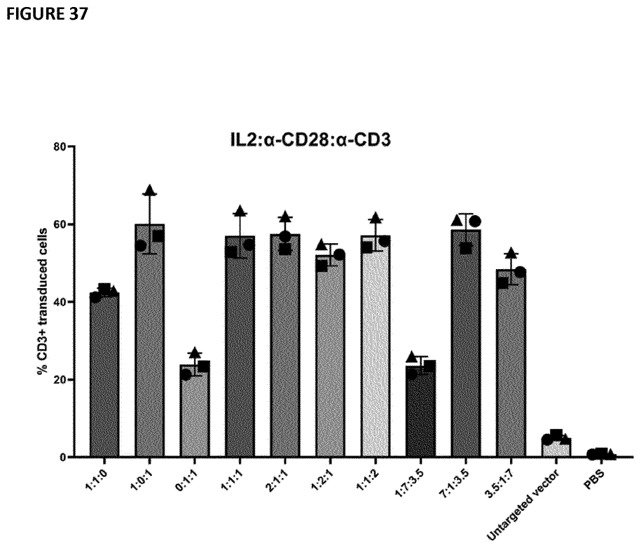

This experiment was performed analogous to Example 6, except that the T cells were pre-activated with dynabeads (Dynabeads Human T-Expander CD3/CD28 (ThermoFisher)). Results are shown in FIG. 37. In pre-activated T cells transfection can also be achieved if the anti-CD28 adapter is not present. Successful transfection can be significantly increased with recombinant adenoviruses displaying an antigen-binding moiety specifically binding to CD3 and a functional interleukin 2 polypeptide.

### Example 30: CD3-Specific Adapters

Using the antibody literature available, scFv adapter proteins with a specificity against CD3 were constructed. In this experiment, scFv's with specificity for human CD3 were selected for the construction of respective adapters.

Nine anti-CD3 scFv's were selected and tested as adapters in the experimental system described herein above. Adapters were also subjected to additional tests, such as mammalian cell production efficiency, SDS-PAGE, size exclusion chromatography, and cell-based binding assays.

Of the initially nine anti-CD3 adapters, the five best ones were further screened in a flow cytometry-assisted cell-based transduction assay with CD3-positive Jurkat cells. Only one constructed improved the transduction efficiency of the adenoviral construct compared to the untargeted vector. The amino acid sequences of anti-CD3 part of this adapter are shown in SEQ ID No.s 11. This adapter has a HCDR1 amino acid sequence of SEQ ID No. 12, a HCDR2 amino acid sequence of SEQ ID No. 13, a HCDR3 amino acid sequence of SEQ ID No. 14, a LCDR1 amino acid sequence of SEQ ID No. 15, a LCDR2 amino acid sequence of SEQ ID No. 16, and a LCDR3 amino acid sequence of SEQ ID No. 17.

1. A recombinant adenovirus displaying a functional interleukin 2 polypeptide.

2. A recombinant adenovirus according to claim 1, wherein said functional interleukin 2 polypeptide is displayed on the knob of said adenovirus.

3. A recombinant adenovirus according to claim 1, wherein said functional interleukin 2 polypeptide is comprised in a recombinant protein comprising from the N- to the C-terminus
a) said functional interleukin 2 polypeptide,
b) a designed ankyrin repeat domain which binds to the knob of the adenovirus, and
c) a trimerization domain.

4. A recombinant adenovirus according to claim 1, wherein said functional interleukin 2 polypeptide comprises the amino acid sequence of SEQ ID No. 4 or 5.

5. A recombinant adenovirus according to claim 3, wherein said designed ankyrin repeat domain that binds to a knob of an adenovirus comprises the amino acid sequence of SEQ ID No. 2.

6. A recombinant adenovirus according to claim 3, wherein said trimerization domain is or is derived from the capsid protein SHP of lambdoid phage 21.

7. A recombinant adenovirus according to claim 6, wherein said trimerization domain comprises the amino acid sequence of SEQ ID No. 1.

8. A method for the transduction of immune cell, said method comprising
a) contacting said immune cell at about the same time with
i) a recombinant adenovirus according to claim 1, and
ii) an agent capable of activating said immune cells, and
b) incubating the mixture obtained in step a) for a time sufficient for transduction of said immune cells.

9. The method according to claim 8, wherein said immune cells are T cells.

10. The method according to claim 9, wherein said T cells are CD4-positive T cells, CD8-positive T cells or Treg cells.

11. The method according to claim 8, wherein said immune cells are NK cells.

12. The method according to claim 8, wherein said agent capable of activating said immune cells is selected from DynaBeads, TransAct, Polybrene and PMA (1-Methoxy-2-propylacetat).

13. The method according to claim 8, wherein said immune cells, said recombinant adenovirus displaying a functional interleukin 2 polypeptide, and said agent capable of activating said immune cells are contacted simultaneously.

14. The method according to claim 8, wherein said method is performed in vitro.

15. The method according to claim 8, wherein said method is performed extra-corporeal.

16. A recombinant adenovirus or a set of recombinant adenoviruses displaying
a) an antigen-binding moiety specifically binding to CD3,
b) an antigen-binding moiety specifically binding to CD28, and
c) a functional interleukin 2 polypeptide.

17. The recombinant adenovirus or the set of recombinant adenoviruses according to claim 16, wherein said adenoviruses are capable of transducing T cells.

18. The recombinant adenovirus or the set of recombinant adenoviruses according to claim 16, wherein said antigen-binding moiety specifically binding to CD3 is a single-chain antibody specifically binding to CD3, preferably wherein said single chain antibody specifically binding to CD3 comprises an HCDR1 of amino acid sequence GYTMN (SEQ ID No. 12), an HCDR2 of amino acid sequence LINPYKGVSTYNQKFKD (SEQ ID No. 13), an HCDR3 of amino acid sequence SGYYGDSDWYFDV (SEQ ID No. 14), an LCDR1 of amino acid sequence RASQDIRNYLN (SEQ ID No. 15), an LCDR2 of amino acid sequence YTSRLES (SEQ ID NO. 16), and an LCDR3 of amino acid sequence QQGNTLPWT (SEQ ID No. 17).

19. The recombinant adenovirus or the set of recombinant adenoviruses according to claim 16, wherein said antigen-binding moiety specifically binding to CD28 is a single-chain antibody specifically binding to CD28, preferably wherein said single chain antibody specifically binding to CD28 comprises an HCDR1 of amino acid sequence SYYIH (SEQ ID No. 19), an HCDR2 of amino acid sequence CIYPGNVNTNYNEKFKD (SEQ ID No. 20), an HCDR3 of amino acid sequence SHYGLDWNEDV (SEQ ID No. 21), an LCDR1 of amino acid sequence HASQNIYVWLN (SEQ ID No. 22), an LCDR2 of amino acid sequence KASNLHT (SEQ ID No. 23), and an LCDR3 of amino acid sequence QQGQTYPYT (SEQ ID No. 24).

20. The recombinant adenovirus or the set of recombinant adenoviruses according to claim 16, wherein said functional interleukin 2 polypeptide comprises the amino acid sequence of SEQ ID No. 3, 4 or 25.

21. The recombinant adenovirus or the set of recombinant adenoviruses according to claim 16, wherein said an antigen-binding moiety specifically binding to CD3, said antigen-binding moiety specifically binding to CD28, and said functional interleukin 2 polypeptide are displayed on the knob of said adenoviruses.

22. The recombinant adenovirus or the set of recombinant adenoviruses according to claim 16, wherein said an antigen-binding moiety specifically binding to CD3, said antigen-binding moiety specifically binding to CD28, and said functional interleukin 2 polypeptide are comprised in a recombinant protein comprising from the N- to the C-terminus
a) said antigen-binding moiety specifically binding to CD3, said antigen-binding moiety specifically binding to CD28, or said functional interleukin 2 polypeptide,
b) a designed ankyrin repeat domain which binds to the knob of the adenovirus, and
c) a trimerization domain.

23. The recombinant adenovirus or the set of recombinant adenoviruses according to claim 22, wherein said designed ankyrin repeat domain that binds to a knob of an adenovirus comprises the amino acid sequence of SEQ ID No. 2.

24. The recombinant adenovirus or the set of recombinant adenoviruses according to claim 22, wherein each of said antigen-binding moieties comprises a polypeptide of SEQ ID No. 35 and a polypeptide of SEQ ID No. 36.

25. The recombinant adenovirus or the set of recombinant adenoviruses according to claim 16, wherein said trimerization domain is or is derived from the capsid protein SHP of lambdoid phage 21, preferably wherein said trimerization domain comprises the amino acid sequence of SEQ ID No. 1.

26. A recombinant protein comprising from the N- to the C-terminus
a) an antigen-binding moiety specifically binding to CD3, an antigen-binding moiety specifically binding to CD28, or a functional interleukin 2 polypeptide,
b) a designed ankyrin repeat domain which binds to the knob of an adenovirus, and
c) a trimerization domain.

27. A trimeric protein consisting of three recombinant proteins according to claim 26.

28. The trimeric protein according to claim 27, wherein said three recombinant proteins are identical, and wherein said recombinant proteins are selected from an antigen-binding moiety specifically binding to CD3, an antigen-binding moiety specifically binding to CD28, and a functional interleukin 2 polypeptide.

29. The trimeric protein according to claim 27, where said three recombinant proteins comprise
a) an antigen-binding moiety specifically binding to CD3 and an antigen-binding moiety specifically binding to CD28,
b) an antigen-binding moiety specifically binding to CD3 and a functional interleukin 2 polypeptide,
c) an antigen-binding moiety specifically binding to CD28 and a functional interleukin 2 polypeptide, or
d) an antigen-binding moiety specifically binding to CD3, an antigen-binding moiety specifically binding to CD28 and a functional interleukin 2 polypeptide.

30. A nucleic acid encoding a recombinant protein according to claim 29.

31. (canceled)


SEQUENCE LISTING (36 sequences)

SEQ ID NO: 1 - protein, 103 aa, lambdoid phage 21
VRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 2 - protein, 157 aa, synthetic construct
RSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQG

SEQ ID NO: 3 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 133 aa, Homo sapiens [sequence omitted]

SEQ ID NO: 5 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 6 - protein, 462 aa, synthetic construct
MKWVTFISLLFLFSSAYSDYKDDDDKHHHHHHGGGGSLEVLFQGPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTKLGGGGSGGGGSGGGGSGGGGSRSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQGVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 7 - protein, 463 aa, synthetic construct
ATMETDTLLLWVLLLWVPGSTGRWSHPQFEKSHHHHHHHHENLYFQSGSPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTKLGGGGSGGGGSGGGGSGGGGSRSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQGVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 8 - protein, 531 aa, synthetic construct
GPGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSKLGGGGSGGGGSGGGGSGGGGSRSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQGVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 9 - protein, 529 aa, synthetic construct
GPGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSKLGGGGSGGGGSGGGGSGGGGSRSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQGVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 10 - protein, 20 aa, synthetic construct
GGGGSGGGGSGGGGSGGGGS

SEQ ID NO: 11 - protein, 251 aa, synthetic construct
GPGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSKL

SEQ ID NO: 12 - protein, 5 aa, synthetic construct
GYTMN

SEQ ID NO: 13 - protein, 17 aa, synthetic construct
LINPYKGVSTYNQKFKD

SEQ ID NO: 14 - protein, 13 aa, synthetic construct
SGYYGDSDWYFDV

SEQ ID NO: 15 - protein, 11 aa, synthetic construct
RASQDIRNYLN

SEQ ID NO: 16 - protein, 7 aa, synthetic construct
YTSRLES

SEQ ID NO: 17 - protein, 9 aa, synthetic construct
QQGNTLPWT

SEQ ID NO: 18 - protein, 249 aa, synthetic construct
GPGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSKL

SEQ ID NO: 19 - protein, 5 aa, synthetic construct
SYYIH

SEQ ID NO: 20 - protein, 17 aa, synthetic construct
CIYPGNVNTNYNEKFKD

SEQ ID NO: 21 - protein, 11 aa, synthetic construct
SHYGLDWNFDV

SEQ ID NO: 22 - protein, 11 aa, synthetic construct
HASQNIYVWLN

SEQ ID NO: 23 - protein, 7 aa, synthetic construct
KASNLHT

SEQ ID NO: 24 - protein, 9 aa, synthetic construct
QQGQTYPYT

SEQ ID NO: 25 - protein, 139 aa, synthetic construct
GPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTKL

SEQ ID NO: 26 - RNA, 20 nt, synthetic construct
  source 1..20: mol_type = other RNA
aaacgcatcctggacccacg

SEQ ID NO: 27 - RNA, 20 nt, synthetic construct
  source 1..20: mol_type = other RNA
agataaaagttcgcatcttc

SEQ ID NO: 28 - RNA, 20 nt, synthetic construct
  source 1..20: mol_type = other RNA
tacactgatacatccctcga

SEQ ID NO: 29 - DNA, 29 bp, synthetic construct
  source 1..29: mol_type = other DNA
gaataacaagtttagaaaccccacggtgg

SEQ ID NO: 30 - DNA, 30 bp, synthetic construct
  source 1..30: mol_type = other DNA
gtttgaccttcgacacctatttgaataccc

SEQ ID NO: 31 - DNA, 22 bp, synthetic construct
  source 1..22: mol_type = other DNA
ggaattgatttgggagagcatc

SEQ ID NO: 32 - DNA, 26 bp, synthetic construct
  source 1..26: mol_type = other DNA
caggtcctggctctacaatttactaa

SEQ ID NO: 33 - DNA, 16 bp, synthetic construct
  source 1..16: mol_type = other DNA
cggcgtgctggacagg

SEQ ID NO: 34 - DNA, 28 bp, synthetic construct
  source 1..28: mol_type = other DNA
agtgtgactgggcagatcatccaccttc

SEQ ID NO: 35 - protein, 22 aa, synthetic construct
GGGGSGGGGSGGGGSGGGGSRS

SEQ ID NO: 36 - protein, 258 aa, synthetic construct
DLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQGVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP